(19) United States
(12) Patent Application Publication
(10) Pub. No.: US 2020/0216441 A1
(43) Pub. Date: Jul. 9, 2020

(54) PESTICIDALLY ACTIVE HETEROCYCLIC DERIVATIVES WITH SULFUR CONTAINING SUBSTITUENTS

(71) Applicant: SYNGENTA PARTICIPATIONS AG, Basel (CH)

(72) Inventors: Sebastian RENDLER, Stein (CH); Andrew EDMUNDS, Stein (CH); Daniel EMERY, Stein (CH); Pierre Joseph Marcel JUNG, Stein (CH); Michel MUEHLEBACH, Stein (CH); Girish RAWAL, Goa (IN); Indira SEN, Goa (IN); Vikas SIKERVAR, Goa (IN)

(73) Assignee: SYNGENTA PARTICIPATIONS AG, Basel (CH)

(21) Appl. No.: 16/608,667

(22) PCT Filed: Apr. 19, 2018
(86) PCT No.: PCT/EP2018/059973

§ 371 Date: Oct. 25, 2019

(30) Foreign Application Priority Data
Apr. 25, 2017 (IN) 201711014675
May 2, 2017 (IN) 201711015514

(51) Int. Cl. C07D 471/04; A01N 43/90

(52) U.S. Cl. CPC C07D 471/04; A01N 43/90

(57) ABSTRACT

Compounds of formula I (I), wherein the substituents are as defined in claim 1, and the agrochemically acceptable salts salts, stereoisomers, enantiomers, tautomers and N-oxides of those compounds, can be used as insecticides and can be prepared in a manner known per se.

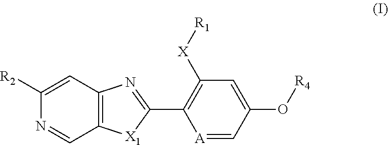

The present invention relates to pesticidally active, in particular insecticidally active heterocyclic derivatives containing sulfur substituents, to intermediates for the preparation of those compounds, to compositions comprising those compounds, and to their use for controlling animal pests (including arthropods and in particular insects or representatives of the order Acarina).

Heterocyclic compounds with pesticidal action are known and described, for example, in WO 2012/086848, WO 2013/018928, WO 2015/000715, and WO 2015/121136.

There have now been found novel pesticidally active heterocyclic 6/5-bicyclic ring derivatives with sulfur containing phenyl and pyridyl substituents.

The present invention accordingly relates to compounds of formula I,

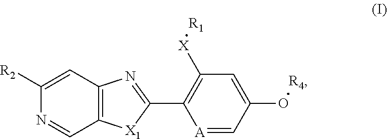

wherein

A is CH or N;

X is S, SO or SO₂;

R₁ is C₁-C₄alkyl, C₁-C₄haloalkyl or C₃-C₆cycloalkylC₁-C₄alkyl;

R₂ is halogen, C₁-C₆haloalkyl, C₁-C₄haloalkylsulfanyl, C₁-C₄haloalkylsulfinyl, C₁-C₄haloalkylsulfonyl or C₁-C₆haloalkoxy;

X₁ is O, S or NR₃, wherein R₃ is C₁-C₄alkyl;

R₄ is C₁-C₆alkyl, C₃-C₆cycloalkylC₁-C₄alkyl, C₃-C₆cyanocycloalkylC₁-C₄alkyl, C₃-C₆halocycloalkyl, C₃-C₆alkynyl, C₃-C₆haloalkenyl, C₃-C₆halocycloalkylC₁-C₄alkyl, C₁-C₄alkoxyC₁-C₄alkyl, C₁-C₄alkoximinoC₁-C₄alkyl, C₁-C₄alkylsulfanylC₁-C₄alkyl, C₁-C₄alkylsulfinylC₁-C₄alkyl, C₁-C₄alkylsulfonylC₁-C₄alkyl, C₁-C₆cyanoalkyl, aminocarbonylC₁-C₄alkyl, C₁-C₄alkylaminocarbonylC₁-C₄alkyl, di(C₁-C₄alkyl)aminocarbonylC₁-C₄alkyl, di(C₁-C₄alkyl)aminocarbonyl, hydroxycarbonylC₁-C₄alkyl, C₁-C₄alkoxycarbonylC₁-C₄alkyl, C₁-C₆cyanohaloalkyl, aminocarbonylC₁-C₄haloalkyl, C₁-C₄alkylaminocarbonylC₁-C₄haloalkyl, di(C₁-C₄alkyl)aminocarbonylC₁-C₄haloalkyl, hydroxycarbonylC₁-C₄haloalkyl, C₁-C₄alkoxycarbonylC₁-C₄haloalkyl, 2,2-difluoroethyl, 2,2,2-trifluoroethyl, 1,3-difluoropropyl, trifluoromethyl, 2-fluoro-1,1-dimethyl-ethyl, 2-fluoroethyl, 1-fluorethyl, 2,2-difluoropropyl, 2-fluoro-1-(fluoromethyl)ethyl, 2,2,2-trifluoro-1,1-dimethyl-ethyl, 1,1-difluoroethyl, di(C₁-C₄alkyl)aminocarbonylC₁-C₄haloalkyl, C₁-C₆cyano(C₁-C₄alkylsulfanyl)alkyl, C₁-C₄alkoxycarbonylC₁-C₄(C₁-C₄alkylsulfonyl)alkyl, C₁-C₄alkoxycarbonylC₁-C₄(C₁-C₄alkylsulfanyl)alkyl, aminocarbonylC₁-C₄(C₁-C₄alkylsulfanyl)alkyl, di(C₁-C₄alkyl)aminocarbonylC₁-C₄(C₁-C₄alkylsulfanyl)alkyl, C₁-C₄alkylaminocarbonylC₁-C₄(C₁-C₄alkylsulfanyl)alkyl, di(C₁-C₄alkyl)aminocarbonylC₁-C₄(C₁-C₄alkylsulfonyl)alkyl, tri-(C₁-C₆)alkylsilyl C₁-C₄alkyl, C1-C6alkylcarbonylC₁-C₄alkyl, imino-C₁-C₄alkyl-oxo-C₁-C₄alkyl-$l\hat{ }{6}-sulfane, C₁-C₄haloalkylsulfonyl, propan-2-imine; or

R₄ is C₃-C₆cycloalkyl which can be mono- or polysubstituted by substituents selected from the group consisting of halogen, cyano, C₁-C₄alkyl, C₁-C₄alkoxy and C₁-C₄haloalkyl; or

R₄ is phenyl or a four- to six-membered heterocyclic ring system which can be partially saturated or fully saturated, said ring system contains 1 to 2 heteroatoms selected from the group consisting of nitrogen, oxygen and sulfur, and said ring system can be mono- or polysubstituted by substituents selected from the group consisting of halogen, cyano, C₁-C₄alkyl, C₃-C₆cycloalkyl, C₁-C₄alkoxy, C₁-C₄haloalkyl and oxo, and where the ring system is attached directly to the oxygen or via an C₁-C₄alkylene chain;

and agrochemically acceptable salts, stereoisomers, enantiomers, tautomers and N-oxides of the compounds of formula I.

Compounds of formula I which have at least one basic centre can form, for example, acid addition salts, for example with strong inorganic acids such as mineral acids, for example perchloric acid, sulfuric acid, nitric acid, nitrous acid, a phosphorus acid or a hydrohalic acid, with strong organic carboxylic acids, such as C₁-C₄alkanecarboxylic acids which are unsubstituted or substituted, for example by halogen, for example acetic acid, such as saturated or unsaturated dicarboxylic acids, for example oxalic acid, malonic acid, succinic acid, maleic acid, fumaric acid or phthalic acid, such as hydroxycarboxylic acids, for example ascorbic acid, lactic acid, malic acid, tartaric acid or citric acid, or such as benzoic acid, or with organic sulfonic acids, such as C₁-C₄alkane- or arylsulfonic acids which are unsubstituted or substituted, for example by halogen, for example methane- or p-toluenesulfonic acid. Compounds of formula I which have at least one acidic group can form, for example, salts with bases, for example mineral salts such as alkali metal or alkaline earth metal salts, for example sodium, potassium or magnesium salts, or salts with ammonia or an organic amine, such as morpholine, piperidine, pyrrolidine, a mono-, di- or tri-lower-alkylamine, for example ethyl-, diethyl-, triethyl- or dimethylpropylamine, or a mono-, di- or trihydroxy-lower-alkylamine, for example mono-, di- or triethanolamine.

The alkyl groups occurring in the definitions of the substituents can be straight-chain or branched and are, for example, methyl, ethyl, n-propyl, isopropyl, n-butyl, sec-butyl, iso-butyl, tert-butyl, pentyl, hexyl, nonyl, decyl and their branched isomers. Alkylsulfanyl, alkylsulfinyl, alkylsulfonyl, alkoxy, alkenyl and alkynyl radicals are derived from the alkyl radicals mentioned. The alkenyl and alkynyl groups can be mono- or polyunsaturated.

Halogen is generally fluorine, chlorine, bromine or iodine. This also applies, correspondingly, to halogen in combination with other meanings, such as haloalkyl or halophenyl.

Haloalkyl groups preferably have a chain length of from 1 to 6 carbon atoms. Haloalkyl is, for example, fluoromethyl, difluoromethyl, trifluoromethyl, chloromethyl, dichloromethyl, trichloromethyl, 2,2,2-trifluoroethyl, 2-fluoroethyl, 2-chloroethyl, 2,2-difluoroethyl, pentafluoroethyl, 1,1-difluoro-2,2,2-trichloroethyl, 2,2,3,3-tetrafluoroethyl and 2,2,2-trichloroethyl.

Haloalkenyl groups are alkenyl groups which are substituted by one or more of the same or different halogen atoms and are, for example, 2,2-difluoro-vinyl or 1,2-dichloro-2-fluoro-vinyl.

Alkoxy groups preferably have a preferred chain length of from 1 to 6 carbon atoms. Alkoxy is, for example, methoxy, ethoxy, propoxy, i-propoxy, n-butoxy, isobutoxy, sec-butoxy and tert-butoxy and also the isomeric pentyloxy and hexyloxy radicals.

Alkylsulfanyl is, for example, methylsulfanyl, ethylsulfanyl, propylsulfanyl, isopropylsulfanyl, n-butylsulfanyl, isobutylsulfanyl, sec-butylsulfanyl or tert-butylsulfanyl; preferably methylsulfanyl or ethylsulfanyl.

Alkylsulfinyl is, for example, methylsulfinyl, ethylsulfinyl, propylsulfinyl, isopropylsulfinyl, n-butylsulfinyl, isobutylsulfinyl, sec-butylsulfinyl or tert-butylsulfinyl; preferably methylsulfinyl or ethylsulfinyl.

Alkylsulfonyl is, for example, methylsulfonyl, ethylsulfonyl, propylsulfonyl, isopropylsulfonyl, n-butylsulfonyl, isobutylsulfonyl, sec-butylsulfonyl or tert-butylsulfonyl; preferably methylsulfonyl or ethylsulfonyl.

Alkylamino is, for example, methylamino, ethylamino, n-propylamino, isopropylamino or the isomeric butylamines. Dialkylamino is, for example, dimethylamino, methylethylamino, diethylamino, n-propylmethylamino, dibutylamino and diisopropylamino. Preference is given to alkylamino groups having a chain length of from 1 to 4 carbon atoms.

Alkoxyalkyl groups preferably have a chain length of 1 to 6 carbon atoms.

Alkoxyalkyl is, for example, methoxymethyl, methoxyethyl, ethoxymethyl, ethoxyethyl, n-propoxymethyl, n-propoxyethyl, isopropoxymethyl or isopropoxyethyl.

The cycloalkyl groups preferably have from 3 to 6 ring carbon atoms, for example cyclopropyl, cyclobutyl, cyclopentyl and cyclohexyl.

According to the present invention, the four- to six-membered heterocyclic ring system which can be partially saturated or fully saturated, said ring system containing 1 to 2 heteroatoms selected from the group consisting of nitrogen, oxygen and sulfur is, depending of the number of ring members, for example, selected from the group consisting of the following heterocyclic groups:

Oxetan-3-yl, oxetan-2-yl, thietan-3-yl, thietan-2-yl, 1-oxothietan-3-yl, 1,1-dioxothietan-3-yl, azetidine-3-yl, tetrahydrofuran-2-yl, tetrahydrofuran-3-yl, tetrahydrothiophen-3-yl, tetrahydrothiophen-2-yl, 1-oxothiolan-3-yl, 1-oxothiolan-2-yl, 1,1-dioxothiolan-3-yl, 1,1-dioxothiolan-2-yl, 4,5-dihydro-isoxazolyl, pyrrolidinyl, [1,3]dioxolanyl, piperidinyl, piperazinyl, [1,4]dioxanyl, imidazolidinyl, [1,3,5]oxadiazinanyl, hexahydro-pyrimidinyl, [1,3,5]triazinanyl and morpholinyl.

In the context of this invention “mono- to polysubstituted” in the definition of the substituents, means typically, depending on the chemical structure of the substituents, monosubstituted to four-times substituted, preferably monosubstituted to three-times substituted, more preferably mono-, or double-substituted.

Free radicals represent methyl groups.

The compounds of formula I according to the invention also include hydrates which may be formed during the salt formation.

Preferred values of R₁, R₂, X, R₄, A and X₁, are, in any combination thereof, as set out below:

Preferably R₁ is C₁-C₄alkyl or C₃-C₆cycloalkyl-C₁-C₄alkyl.

More preferably R₁ is C₁-C₄alkyl or cyclopropylmethyl.

Even more preferably R₁ is methyl, ethyl, n-propyl, i-propyl or cyclopropylmethyl.

Most preferably R₁ is ethyl.

Preferably R₂ is halogen, C₁-C₄haloalkyl, C₁-C₄haloalkylsulfanyl, C₁-C₄haloalkylsulfinyl or C₁-C₄haloalkylsulfonyl.

More preferably R₂ is halogen or C₁-C₃haloalkyl.

Even more preferably R₂ is C₁-C₂haloalkyl.

Most preferably R₂ is trifluoromethyl.

Preferably R₄ is C₃-C₆cycloalkylC₁-C₄alkyl, C₃-C₆cyanocycloalkylC₁-C₄alkyl, C₃-C₆halocycloalkyl, C₃-C₆halocycloalkylC₁-C₄alkyl, di(C₁-C₄alkyl)aminocarbonylC₁-C₄haloalkyl, C₁-C₄alkylsulfanylC₁-C₄alkyl, C₁-C₄alkylsulfonylC₁-C₄alkyl, isopropyl, tert-butyl, 2,2-difluoroethyl, 2,2,2-trifluoroethyl, 2,2-difluoropropyl, 2-fluoroethyl, 1-fluoroethyl, —C(CH₃)₂CN, oxetanyl, 1,3-difluoropropyl or —CH₂CH₂OCH₃.

More preferably R₄ is C₃-C₄cycloalkylC₁-C₂alkyl, C₃-C₄cyanocycloalkylC₁-C₂alkyl, C₃-C₄halocycloalkyl C₃-C₄halocycloalkylC₁-C₂alkyl, di(C₁-C₂alkyl)aminocarbonylC₁-C₂haloalkyl, C₁-C₂alkylsulfanylC₁-C₄alkyl, C₁-C₂alkylsulfonylC₁-C₄alkyl, isopropyl, tert-butyl, 2,2-difluoroethyl, 2,2,2-trifluoroethyl, 2,2-difluoropropyl, 2-fluoroethyl, 1-fluoroethyl, —C(CH₃)₂CN, oxetanyl, 1,3-difluoropropyl or —CH₂CH₂OCH₃.

Even more preferably R₄ is cyclopropylmethyl, 1-cyanocyclopropylmethyl, 3,3-difluorocyclobutyl, dimethylaminocarbonyl-CF₂—, methylsulfanylC₁-C₄alkyl, methylsulfonylC₁-C₄alkyl, isopropyl, tert-butyl, 2,2-difluoroethyl, 2,2,2-trifluoroethyl, 2,2-difluoropropyl, 2-fluoroethyl, 1-fluoroethyl, —C(CH₃)₂CN, oxetan-3-yl, 1,3-difluoropropyl or —CH₂CH₂OCH₃.

Most preferably R₄ is 1-cyanocyclopropylmethyl, 3,3-difluorocyclobutyl, methylsulfanylC₁-C₄alkyl, methylsulfonylC₁-C₄alkyl, —C(CH₃)₂CN, or 1,3-difluoroprop-2-yl.

Preferably X is S or SO₂.

Most preferably X is SO₂.

Preferably X₁ is NR₃, wherein R₃ is C₁-C₄alkyl.

Most preferably X₁ is NCH₃.

Preferably A is N.

Embodiments according to the invention are provided as set out below.

Embodiment 1 provides compounds of formula I, or an agrochemically acceptable salt, stereoisomer, enantiomer, tautomer or N-oxide thereof, as defined above.

Embodiment 2 provides compounds, or an agrochemically acceptable salt, stereoisomer, enantiomer, tautomer or N-oxide thereof, according to embodiment 1 wherein R₁ is C₁-C₄alkyl or C₃-C₆cycloalkyl-C₁-C₄alkyl.

Embodiment 3 provides compounds, or an agrochemically acceptable salt, stereoisomer, enantiomer, tautomer or N-oxide thereof, according to embodiment 1 or 2 wherein R₂ is halogen, C₁-C₄haloalkyl, C₁-C₄haloalkylsulfanyl, C₁-C₄haloalkylsulfinyl or C₁-C₄haloalkylsulfonyl.

Embodiment 4 provides compounds, or an agrochemically acceptable salt, stereoisomer, enantiomer, tautomer or N-oxide thereof, according to any one of embodiments 1, 2 or 3 wherein R₄ is C₃-C₆cycloalkylC₁-C₄alkyl, C₃-C₆cyanocycloalkylC₁-C₄alkyl, C₃-C₆halocycloalkyl, C₃-C₆halocycloalkylC₁-C₄alkyl, di(C₁-C₄alkyl)aminocarbonylC₁-C₄haloalkyl, C₁-C₄alkylsulfanylC₁-C₄alkyl, C₁-C₄alkylsulfinylC₁-C₄alkyl, C₁-C₄alkylsulfonylC₁-C₄alkyl, isopropyl, tert-butyl, 2,2-difluoroethyl, 2,2,2-trifluoroethyl, 2,2-difluoropropyl, 2-fluoroethyl, 1-fluoroethyl, —C(CH₃)₂CN, oxetanyl, 1,3-difluoropropyl or —CH₂CH₂OCH₃.

Embodiment 5 provides compounds, or an agrochemically acceptable salt, stereoisomer, enantiomer, tautomer or N-oxide thereof, according to any one of embodiments 1, 2, 3 or 4 wherein R₁ is C₁-C₄alkyl or cyclopropylmethyl.

Embodiment 6 provides compounds, or an agrochemically acceptable salt, stereoisomer, enantiomer, tautomer or N-oxide thereof, according to any one of embodiments 1, 2, 3, 4, or 5 wherein R₂ is halogen or C₁-C₃haloalkyl.

Embodiment 7 provides compounds, or an agrochemically acceptable salt, stereoisomer, enantiomer, tautomer or N-oxide thereof, according to any one of embodiments 1, 2, 3, 4, 5, or 6 wherein R₄ is C₃-C₄cycloalkylC₁-C₂alkyl, C₃-C₄cyanocycloalkylC₁-C₂alkyl, C₃-C₄halocycloalkyl C₃-C₄halocycloalkylC₁-C₂alkyl, di(C₁-C₂alkyl)aminocarbonylC₁-C₂haloalkyl, C₁-C₂alkylsulfanylC₁-C₄alkyl, C₁-C₂alkylsulfinylC₁-C₄alkyl, C₁-C₂alkylsulfonylC₁-C₄alkyl, isopropyl, tert-butyl, 2,2-difluoroethyl, 2,2,2-trifluoroethyl, 2,2-difluoropropyl, 2-fluoroethyl, 1-fluoroethyl, —C(CH₃)₂CN, oxetanyl, 1,3-difluoropropyl or —CH₂CH₂OCH₃.

Embodiment 8 provides compounds, or an agrochemically acceptable salt, stereoisomer, enantiomer, tautomer or N-oxide thereof, according to any one of embodiments 1, 2, 3, 4, 5, 6, or 7 wherein R₁ is methyl, ethyl, n-propyl, i-propyl or cyclopropylmethyl.

Embodiment 9 provides compounds, or an agrochemically acceptable salt, stereoisomer, enantiomer, tautomer or N-oxide thereof, according to any one of embodiments 1, 2, 3, 4, 5, 6, 7, or 8 wherein R₂ is C₁-C₂haloalkyl.

Embodiment 10 provides compounds, or an agrochemically acceptable salt, stereoisomer, enantiomer, tautomer or N-oxide thereof, according to any one of embodiments 1, 2, 3, 4, 5, 6, 7, 8, or 9 wherein R₄ is cyclopropylmethyl, 1-cyanocyclopropylmethyl, 3,3-difluorocyclobutyl, dimethylaminocarbonyl-CF₂—, methylsulfanylC₁-C₄alkyl, methylsulfonylC₁-C₄alkyl, isopropyl, tert-butyl, 2,2-difluoroethyl, 2,2,2-trifluoroethyl, 2,2-difluoropropyl, 2-fluoroethyl, 1-fluoroethyl, —C(CH₃)₂CN, oxetan-3-yl, 1,3-difluoropropyl or —CH₂CH₂OCH₃.

Embodiment 11 provides compounds, or an agrochemically acceptable salt, stereoisomer, enantiomer, tautomer or N-oxide thereof, according to any one of embodiments 1, 2, 3, 4, 5, 6, 7, 8, 9 or 10 wherein X is S or SO₂.

Embodiment 12 provides compounds, or an agrochemically acceptable salt, stereoisomer, enantiomer, tautomer or N-oxide thereof, according to any one of embodiments 1, 2, 3, 4, 5, 6, 7, 8, 9, 10 or 11 wherein X₁ is NR₃, wherein R₃ is C₁-C₄alkyl.

Embodiment 13 provides compounds, or an agrochemically acceptable salt, stereoisomer, enantiomer, tautomer or N-oxide thereof, according to any one of embodiments 1, 2, 3, 4, 5, 6, 7, 8, 9, 10, 11 or 12 wherein R₁ is ethyl.

Embodiment 14 provides compounds, or an agrochemically acceptable salt, stereoisomer, enantiomer, tautomer or N-oxide thereof, according to any one of embodiments 1, 2, 3, 4, 5, 6, 7, 8, 9, 10, 11, 12 or 13 wherein R₂ is trifluoromethyl.

Embodiment 15 provides compounds, or an agrochemically acceptable salt, stereoisomer, enantiomer, tautomer or N-oxide thereof, according to any one of embodiments 1, 2, 3, 4, 5, 6, 7, 8, 9, 10, 11, 12, 13 or 14 wherein R₄ is 1-cyanocyclopropylmethyl, 3,3-difluorocyclobutyl, methylsulfanylC₁-C₄alkyl, methylsulfonylC₁-C₄alkyl, —C(CH₃)₂CN, or 1,3-difluoroprop-2-yl.

Embodiment 16 provides compounds, or an agrochemically acceptable salt, stereoisomer, enantiomer, tautomer or N-oxide thereof, according to any one of embodiments 1, 2, 3, 4, 5, 6, 7, 8, 9, 10, 11, 12, 13, 14 or 15 wherein X is SO₂.

Embodiment 17 provides compounds, or an agrochemically acceptable salt, stereoisomer, enantiomer, tautomer or N-oxide thereof, according to any one of embodiments 1, 2, 3, 4, 5, 6, 7, 8, 9, 10, 11, 12, 13, 14, 15 or 16 wherein X₁ is NCH₃.

Embodiment 18 provides compounds, or an agrochemically acceptable salt, stereoisomer, enantiomer, tautomer or N-oxide thereof, according to any one of embodiments 1, 2, 3, 4, 5, 6, 7, 8, 9, 10, 11, 12, 13, 14, 15, 16 or 17 wherein A is CH.

Embodiment 19 provides compounds, or an agrochemically acceptable salt, stereoisomer, enantiomer, tautomer or N-oxide thereof, according to any one of embodiments 1, 2, 3, 4, 5, 6, 7, 8, 9, 10, 11, 12, 13, 14, 15, 16 or 17 wherein A is N.

A preferred group of compound of formula I are compounds of formula (I′),

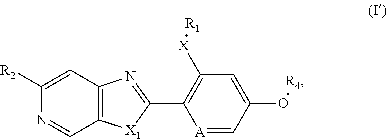

wherein

A is CH or N;

X is S, SO or SO₂;

R₁ is C₁-C₄alkyl, C₁-C₄haloalkyl or C₃-C₆cycloalkylC₁-C₄alkyl;

R₂ is halogen, C₁-C₆haloalkyl, C₁-C₄haloalkylsulfanyl, C₁-C₄haloalkylsulfinyl, C₁-C₄haloalkylsulfonyl or C₁-C₆haloalkoxy;

X₁ is O, S or NR₃, wherein R₃ is C₁-C₄alkyl;

R₄ is C₁-C₆alkyl, C₃-C₆cycloalkylC₁-C₄alkyl, C₃-C₆alkynyl, C₃-C₆haloalkenyl, C₃-C₆halocycloalkylC₁-C₄alkyl, C₁-C₄alkoxyC₁-C₄alkyl, C₁-C₄alkoximinoC₁-C₄alkyl, C₁-C₄alkylsulfanylC₁-C₄alkyl, C₁-C₄alkylsulfinylC₁-C₄alkyl, C₁-C₄alkylsulfonylC₁-C₄alkyl, C₁-C₆cyanoalkyl, aminocarbonylC₁-C₄alkyl, C₁-C₄alkylaminocarbonylC₁-C₄alkyl, di(C₁-C₄alkyl)aminocarbonylC₁-C₄alkyl, hydroxycarbonylC₁-C₄alkyl, C₁-C₄alkoxycarbonylC₁-C₄alkyl, C₁-C₆cyanohaloalkyl, aminocarbonylC₁-C₄haloalkyl, C₁-C₄alkylaminocarbonylC₁-C₄haloalkyl, di(C₁-C₄alkyl)aminocarbonylC₁-C₄haloalkyl, hydroxycarbonylC₁-C₄haloalkyl, C₁-C₄alkoxycarbonylC₁-C₄haloalkyl, 2,2-difluoroethyl, 2,2,2-trifluoroethyl; or is C₃-C₆cycloalkyl which can be mono- or polysubstituted by substituents selected from the group consisting of halogen, cyano, C₁-C₄alkyl, C₁-C₄alkoxy and C₁-C₄haloalkyl; or

R₄ is a four- to six-membered heterocyclic ring system which can be partially saturated or fully saturated, said ring system contains 1 to 2 heteroatoms selected from the group consisting of nitrogen, oxygen and sulfur, and said ring system can be mono- or polysubstituted by substituents selected from the group consisting of halogen, cyano, C₁-C₄alkyl, C₁-C₄alkoxy, C₁-C₄haloalkyl and oxo; and agrochemically acceptable salts, stereoisomers, enantiomers, tautomers and N-oxides of the compounds of formula I.

A preferred group of compounds of formula I is represented by the compounds of formula I-1

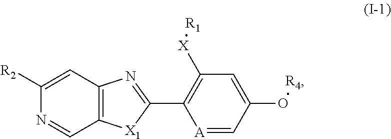

wherein A, X, R₁, R₂, and X₁ are as defined under formula I above and

R₄ is C₁-C₆alkyl, C₃-C₆cycloalkylC₁-C₄alkyl, C₃-C₆alkynyl, C₃-C₆haloalkenyl, C₃-C₆halocycloalkylC₁-C₄alkyl, C₁-C₄alkoxyC₁-C₄alkyl, C₁-C₄alkoximinoC₁-C₄alkyl, C₁-C₄alkylsulfanylC₁-C₄alkyl, C₁-C₄alkylsulfinylC₁-C₄alkyl, C₁-C₄alkylsulfonylC₁-C₄alkyl, C₁-C₆cyanoalkyl, aminocarbonylC₁-C₄alkyl, C₁-C₄alkylaminocarbonylC₁-C₄alkyl, di(C₁-C₄alkyl)aminocarbonylC₁-C₄alkyl, hydroxycarbonylC₁-C₄alkyl, C₁-C₄alkoxycarbonylC₁-C₄alkyl, C₁-C₆cyanohaloalkyl, aminocarbonylC₁-C₄haloalkyl, C₁-C₄alkylaminocarbonylC₁-C₄haloalkyl, di(C₁-C₄alkyl)aminocarbonylC₁-C₄haloalkyl, hydroxycarbonylC₁-C₄haloalkyl, C₁-C₄alkoxycarbonylC₁-C₄haloalkyl; or is C₃-C₆cycloalkyl which can be mono- or polysubstituted by substituents selected from the group consisting of halogen, cyano, C₁-C₄alkyl, C₁-C₄alkoxy, C₁-C₄haloalkyl, or R₄ is a four- to six-membered heterocyclic ring system which can be partially saturated or fully saturated, said ring system contains 1 to 2 heteroatoms selected from the group consisting of nitrogen, oxygen and sulfur, and said ring system can be mono- or polysubstituted by substituents selected from the group consisting of halogen, cyano, C₁-C₄alkyl, C₁-C₄alkoxy, C₁-C₄haloalkyl and oxo.

In said preferred group of compounds of formula I-1,

R₁ is preferably methyl, ethyl, n-propyl, i-propyl or cyclopropylmethyl;

R₂ is preferably C₁-C₄haloalkyl, C₁-C₄haloalkylsulfanyl, C₁-C₄haloalkylsulfinyl or C₁-C₄haloalkylsulfonyl;

X is preferably SO₂ and X₁ is preferably NR₃, wherein R₃ is C₁-C₄alkyl.

Especially preferred compounds of formula I-1 are those, wherein R₁ is ethyl and X is SO₂.

A preferred group of compounds of formula I is represented by the compounds of formula I-2

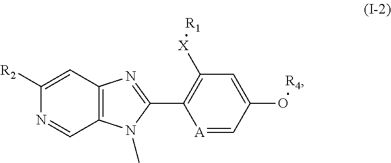

wherein A, X, R₁, and R₂ are defined under formula I above and R₄ is defined as under formula I-1. In said preferred group of compounds of formula I-2,

R₁ is preferably methyl, ethyl, n-propyl, i-propyl or cyclopropylmethyl;

R₂ is preferably C₁-C₄haloalkyl, C₁-C₄haloalkylsulfanyl, C₁-C₄haloalkylsulfinyl or C₁-C₄haloalkylsulfonyl; and X is preferably SO₂.

Especially preferred compounds of formula I-2 are those, wherein R₁ is ethyl and X is SO₂.

A more preferred group of compounds of formula I-2 is represented by the compounds of formula I-2a

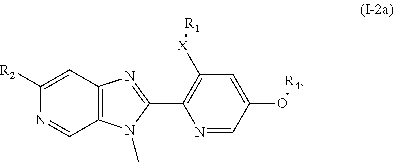

wherein X, R₁, and R₂ are defined under formula I above and R₄ is defined as under formula I-1. In said preferred group of compounds of formula I-2a,

R₁ is preferably methyl, ethyl, n-propyl, i-propyl or cyclopropylmethyl;

R₂ is preferably C₁-C₄haloalkyl, C₁-C₄haloalkylsulfanyl, C₁-C₄haloalkylsulfinyl or C₁-C₄haloalkylsulfonyl; and X is preferably SO₂.

Especially preferred compounds of formula I-2a are those, wherein R₁ is ethyl and X is SO₂.

Another more preferred group of compounds of formula I-2 is represented by the compounds of formula I-2b

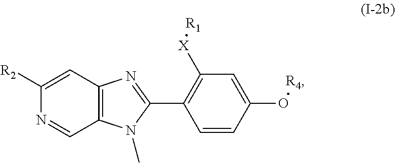

wherein X, R₁, and R₂ are defined under formula I above and R₄ is defined as under formula I-1. In said preferred group of compounds of formula I-2b,

R₁ is preferably methyl, ethyl, n-propyl, i-propyl or cyclopropylmethyl;

R₂ is preferably C₁-C₄haloalkyl, C₁-C₄haloalkylsulfanyl, C₁-C₄haloalkylsulfinyl or C₁-C₄haloalkylsulfonyl; and X is preferably SO₂.

Especially preferred compounds of formula I-2b are those, wherein R₁ is ethyl and X is SO₂.

A further preferred group of compounds of formula I is represented by the compounds of formula I-3

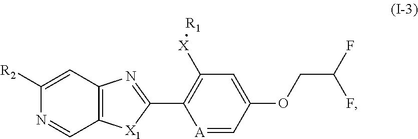

wherein A, X, X₁, R₁, and R₂ are as defined under formula I above. In said preferred group of compounds of formula I-3,

R₁ is preferably methyl, ethyl, n-propyl, i-propyl or cyclopropylmethyl;

R₂ is preferably C₁-C₄haloalkyl, C₁-C₄haloalkylsulfanyl, C₁-C₄haloalkylsulfinyl or C₁-C₄haloalkylsulfonyl; and X is preferably SO₂.

Especially preferred compounds of formula I-3 are those, wherein R₁ is ethyl and X is SO₂.

A more preferred group of compounds of formula I-3 is represented by the compounds of formula I-3a

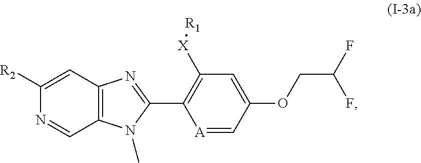

wherein A, X, R₁, and R₂ are as defined under formula I above. In said preferred group of compounds of formula I-3a,

R₁ is preferably methyl, ethyl, n-propyl, i-propyl or cyclopropylmethyl;

R₂ is preferably C₁-C₄haloalkyl, C₁-C₄haloalkylsulfanyl, C₁-C₄haloalkylsulfinyl or C₁-C₄haloalkylsulfonyl; and X is preferably SO₂.

Especially preferred compounds of formula I-3a are those, wherein R₁ is ethyl and X is SO₂.

A further preferred group of compounds of formula I is represented by the compounds of formula I-4

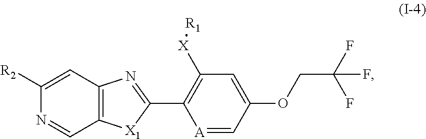

wherein A, X, X₁, R₁, and R₂ are as defined under formula I above. In said preferred group of compounds of formula I-4,

R₁ is preferably methyl, ethyl, n-propyl, i-propyl or cyclopropylmethyl;

R₂ is preferably C₁-C₄haloalkyl, C₁-C₄haloalkylsulfanyl, C₁-C₄haloalkylsulfinyl or C₁-C₄haloalkylsulfonyl; and X is preferably SO₂.

Especially preferred compounds of formula I-4 are those, wherein R₁ is ethyl and X is SO₂.

A more preferred group of compounds of formula I-4 is represented by the compounds of formula I-4a

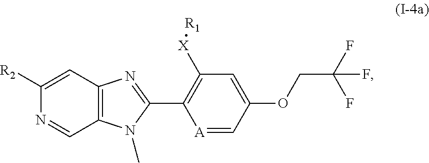

wherein A, X, R₁, and R₂ are as defined under formula I above. In said preferred group of compounds of formula I-4a,

R₁ is preferably methyl, ethyl, n-propyl, i-propyl or cyclopropylmethyl;

R₂ is preferably C₁-C₄haloalkyl, C₁-C₄haloalkylsulfanyl, C₁-C₄haloalkylsulfinyl or C₁-C₄haloalkylsulfonyl; and X is preferably SO₂.

Especially preferred compounds of formula I-4a are those, wherein R₁ is ethyl and X is SO₂.

In particular preferred compounds of formula I,

R₁ is C₁-C₄alkyl, preferably ethyl;

R₂ is C₁-C₄haloalkyl, preferably trifluoromethyl;

X₁ is NR₃; wherein R₃ is C₁-C₄alkyl; preferably methyl;

A is CH or N;

X is S or SO₂; and

R₄ is oxetanyl which can be mono-substituted by C₁-C₄alkyl, or is C₁-C₄alkyl which can be substituted by cyano, or is C₃-C₆alkynyl, 2,2-difluoroethyl, 2,2,2-trifluoroethyl, C₃-C₆cycloalkyl-C₁-C₄alkyl, di(C₁-C₄alkyl)aminocarbonylC₁-C₄haloalkyl, C₁-C₄alkylsulfonyl-C₁-C₄alkyl, C₁-C₄alkoxy-C₁-C₄alkyl or C₁-C₄alkylthio-C₁-C₄alkyl;

preferably oxetanyl which can be mono-substituted by C₁-C₄alkyl, C₁-C₄alkyl, diethylaminocarbonyl-C₁-C₄haloalkyl, methoxy-C₁-C₄alkyl, methylthio-C₁-C₄alkyl, 2,2,2-trifluoroethyl, 2,2-difluoroethyl, propargyl or cyclopropyl-C₁-C₄alkyl.

In another particular preferred compounds of formula I,

R₁ is C₁-C₄alkyl, preferably ethyl;

R₂ is C₁-C₄haloalkyl, preferably trifluoromethyl;

X₁ is NR₃; wherein R₃ is C₁-C₄alkyl; preferably methyl;

A is CH or N;

X is S or SO₂; and

R₄ is oxetanyl which can be mono-substituted by C₁-C₄alkyl, or is C₁-C₄alkyl which can be substituted by cyano, or is C₃-C₆alkynyl, C₃-C₆cycloalkyl-C₁-C₄alkyl, di(C₁-C₄alkyl)aminocarbonylC₁-C₄haloalkyl, C₁-C₄alkylsulfonyl-C₁-C₄alkyl, C₁-C₄alkoxy-C₁-C₄alkyl or C₁-C₄alkylthio-C₁-C₄alkyl; preferably oxetanyl which can be mono-substituted by C₁-C₄alkyl, C₁-C₄alkyl, diethylaminocarbonyl-C₁-C₄haloalkyl, methoxy-C₁-C₄alkyl, methylthio-C₁-C₄alkyl, propargyl or cyclopropyl-C₁-C₄alkyl.

The process according to the invention for preparing compounds of formula I is carried out by methods known to those skilled in the art. Compounds of formula I-a3, wherein X is SO₂ and A, X₁, R₁, R₂, and R₄ are defined as under formula I above, may be prepared by oxidation of compounds of formula I-a2, wherein X is SO and A, X₁, R₁, R₂, and R₄ are defined as under formula I above. The reaction can be performed with reagents such as a peracid, for example peracetic acid or m-chloroperbenzoic acid, or a hydroperoxide, as for example, hydrogen peroxide or tert-butylhydroperoxide, or an inorganic oxidant, such as a monoperoxo-disulfate salt or potassium permanganate. In a similar way, compounds of formula I-a2, wherein A, X₁, R₁, R₂, and R₄ are defined as under formula I above, may be prepared by oxidation of compounds of formula I-a1, wherein X is S and A, X₁, R₁, R₂, and R₄ are defined as under formula I above, under analogous conditions described above. These reactions can be performed in various organic or aqueous solvents compatible to these conditions, by temperatures from below 0° C. up to the boiling point of the solvent system. The transformation of compounds of the formula I-a1 into compounds of the formula I-a2 and 1-a3 is represented in Scheme 1.

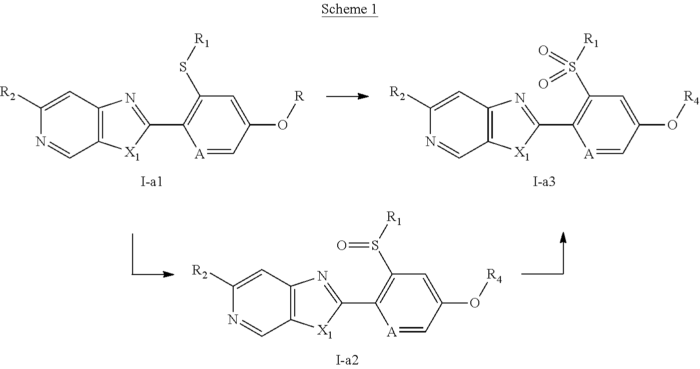

Compounds of formula I can be prepared by various methods from compounds of formula II, wherein X, A, X₁, R₁ and R₂ are defined as under formula I and Xₐ₁ is halogen, preferably, I, Br or Cl, or OTf (Scheme 2). For example, compounds of formula I can be obtained via palladium-catalyzed ether formation with alcohols of formula VI, wherein R₄ is defined as under formula I, in the presence of a Pd-complex, such as commercially available {(R)-1-[(Sp)-2-(Dicyclohexylphosphino)ferrocenyl]ethyldi-tert-butylphosphine}[2-(2′-amino-1,1′-biphenyl)]palladium(II) methanesulfonate (Josiphos SL-J009-1 Pd G3) and a base, such as K₂CO₃ or Cs₂CO₃ in a solvent such a s toluene, tetrahydrofuran, or 1,4-dioxane at temperatures between 25 and 120° C. Alternatively, compounds of formula II can be converted to compounds of formula IV, wherein X, A, X₁, R₁ and R₂ are defined as under formula I via palladium-catalyzed hydroxylation in the presence of a palladium source, such as Pd(dba)₂, and a phosphine ligand, such as tBu-XPhos (CAS 564483-19-8) in the presence of a hydroxide salt, such as CsOH, NaOH or KOH, and in a solvent such as 1,4-dioxane, or THF at temperatures between 20-120° C. Compounds of formula IV can be treated with alkylating reagents of general formula V wherein Xa₂ is preferably Br, I, or OTf and R₄ is defined under formula I, in the presence of a base, such as sodium hydride, K₂CO₃, or Cs₂CO₃, in an inert solvent such as THF, DMF, or acetonitrile, to give compounds of formula I. Compounds of formula IV can be also obtained by Miyura borylation of compounds of formula II. The intermediate of formula III, wherein A, X₁, X, R₁ and R₂, are defined as under formula I and BL₂ stands for a boronic acid derivative, preferably 4,4,5,5-tetramethyl-1,3,2-dioxaborolan-2-yl, can be obtained by treatment of compounds of formula II, in a palladium catalyzed reaction with bispinacol diborane (BPin)₂. This reaction, can be performed in an aprotic solvent, in presence of a base, preferably a weak base, such as potassium acetate KOAc, and Pd(dppf)Cl₂ as a common catalyst for this type of reaction. The temperature of the reaction is preferably performed between 0° C. and the boiling point of the reaction mixture. The obtained intermediate III can be converted to compounds of formula IV by treatment with an oxidizing reagent, preferably hydrogen peroxide or the urea complex thereof. Compounds of formula II are known and syntheses have been previously described, e.g. in WO 2016/030229.

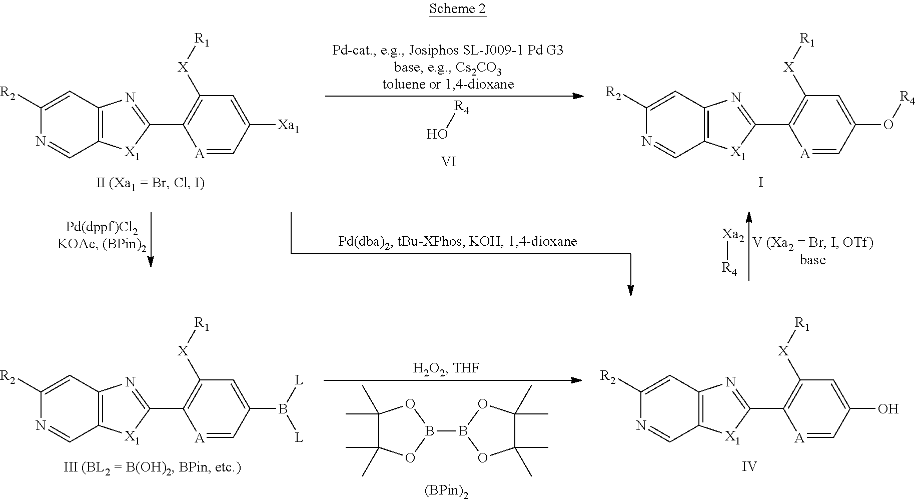

Alternatively, compounds of formula I may be prepared from compounds of formula VII, wherein A, X₁, R₁ and R₂ are defined as under formula I (Scheme 3). Treatment of compounds of formula VII, wherein R₁, A, X and R₄ are defined as above for formula I, can be activated to compounds of formula VIII by methods known to those skilled in the art and described in for example Tetrahedron 2005, 61 (46), 10827-10852. For example compounds of formula VIII where X₀ is chlorine are formed by treatment with for example, oxalyl chloride or thionyl chloride in the presence of catalytic quantities of DMF in inert solvents such as methylene chloride or THF at temperatures between 20° C. to 100° C., preferably 25° C. Treatment of VIII with compounds of formula IX, wherein R₂ and X₁ are defined as described in formula I, optionally in the presence of a base, e.g. triethylamine or pyridine, leads to compounds of formula X, wherein R₂, X₁, X, R₁ and R₄ are defined as in formula I. Alternatively, compounds of formula VIII can be prepared by treatment of compounds of formula VII with dicyclohexyl carbodiimide (DCC) or 1-ethyl-3-(3-dimethylaminopropyl)carbodiimide (EDC) to give the activated species VIII, wherein X₀ is X₀₁ and X₀₂, respectively, in an inert solvent, e.g. pyridine, or tetrahydrofuran (THF) optionally in the presence of a base, e.g., triethylamine, at temperatures between 50-180° C. The obtained compounds of formula X can then be converted to compounds of formula I by dehydration, e.g., by heating under microwave irradiation, in the presence of an acid catalyst, for example methanesulfonic acid, or para-toluenesulfonic acid, in an inert solvent such as N-methyl pyrrolidone or toluene at temperatures between 25-180° C., preferably 130-170° C. Such processes have been described previously in WO 2010/125985. Alternatively, compounds of formula X can be converted to compounds of formula I (wherein X₁ is O) using triphenylphosphine, di-isopropyl azodicarboxylate in an inert solvent such as THF at temperatures between 25-50° C. Such Mitsunobu conditions have been previously described for such transformations (see WO 2009/131237). Compounds of formula IX have been previously described, e.g., in WO 2015/000715 and WO 2016/116338.

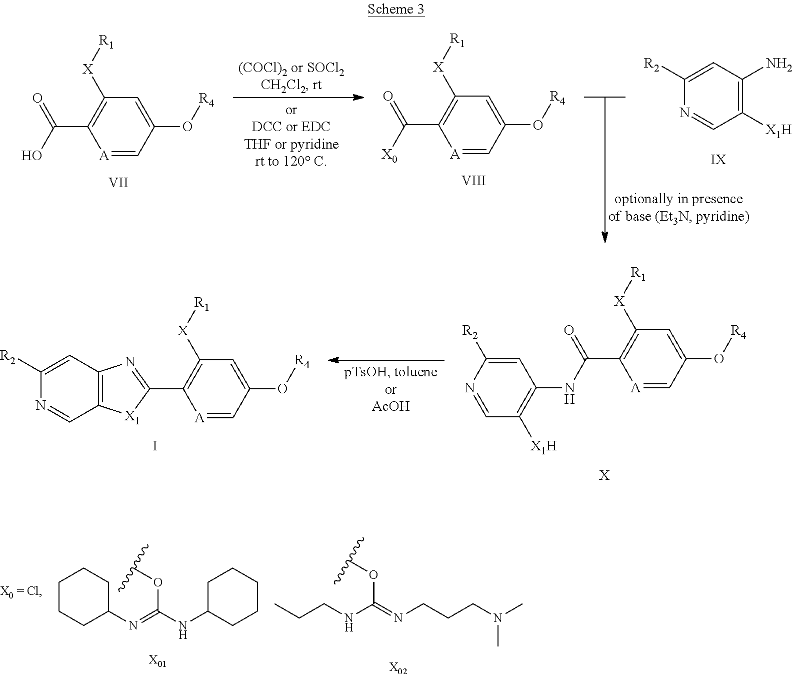

Compounds of formula VII, wherein R₁, A, X and R₄ are defined as above for formula I, can be prepared by methods described in Scheme 4. Typically, compounds of formula VII can be obtained from compound of formula XIV, wherein A, X, R₁ and R₄ are defined as under formula I and R_{LG} is C₁-C₄alkyl, by saponification under conditions known to a person skilled in the art. Compounds of formula XIV are available by various methods, for example from compounds of formula XI, wherein R₁, X, A are as defined as above for formula I, and wherein X_{b1} can be a halogen, preferentially chlorine, bromine or iodine, or a sulfonate, like for example a trifluoromethanesulfonate, most preferably bromine or iodine, and R_{LG} is C₁-C₄alkyl as detailed in Scheme 4. Compounds of formula XI have been previously described, for example in WO 2016/026848. Such compounds of formula XI can subjected to palladium-catalyzed ether formation with alcohols of formula VI, wherein R₄ is defined as under formula I, in the presence of a Pd-complex, such as commercially available {(R)-1-[(Sp)-2-(Dicyclohexyl-phosphino)ferrocenyl]ethyldi-tert-butylphosphine}[2-(2′-amino-1,1′-biphenyl)]palladium(II) methanesulfonate (Josiphos SL-J009-1 Pd G3) and a base, such as K₂CO₃ or Cs₂CO₃ in a solvent such a s toluene, tetrahydrofuran, or 1,4-dioxane at temperatures between 25 and 120° C. to give compounds of formula XIV. Alternatively, compounds of formula XI can be converted to compounds of formua XIII, wherein X, A, X₁, and R₁ are defined as under formula I and R_{LG} is C₁-C₄alkyl, via palladium-catalyzed hydroxylation in the presence of a palladium source, such as Pd(dba)₂, and a phosphine ligand, such as tBu-XPhos (CAS 564483-19-8) in the presence of a hydroxide salt, such as CsOH, NaOH or KOH, and in a solvent such as 1,4-dioxane, or THF at temperatures between 20-120° C. Compounds of formula XIII can be treated with alkylating reagents of general formula V wherein Xb₂ is preferably Br, I, or OTf and R₄ is defined under formula I, in the presence of a base, such as sodium hydride, K₂CO₃, or Cs₂CO₃, in an inert solvent such as THF, DMF, or acetonitrile, to give compounds of formula XIVI. Compounds of formula XIII can be also obtained by Miyura borylation of compounds of formula XI. The intermediate of formula XII, wherein A, X, R₁ are defined as under formula I, R_{LG} is C₁-C₄alkyl, and BL₂ stands for a boronic acid derivative, preferably 4,4,5,5-tetramethyl-1,3,2-dioxaborolan-2-yl, can be obtained by treatment of compounds of formula XI, in a palladium catalyzed reaction with bispinacol diborane (BPin)₂. This reaction, can be performed in an aprotic solvent, in presence of a base, preferably a weak base, such as potassium acetate KOAc, and Pd(dppf)Cl₂ as a common catalyst for this type of reaction. The temperature of the reaction is preferably performed between 0° C. and the boiling point of the reaction mixture. The obtained intermediate XII can be converted to compounds of formula XIII by treatment with an oxidizing reagent, preferably hydrogen peroxide or the urea complex thereof. Compounds of formula V and VI are commercially available or methods to prepare those compounds are known to a person skilled in the art.

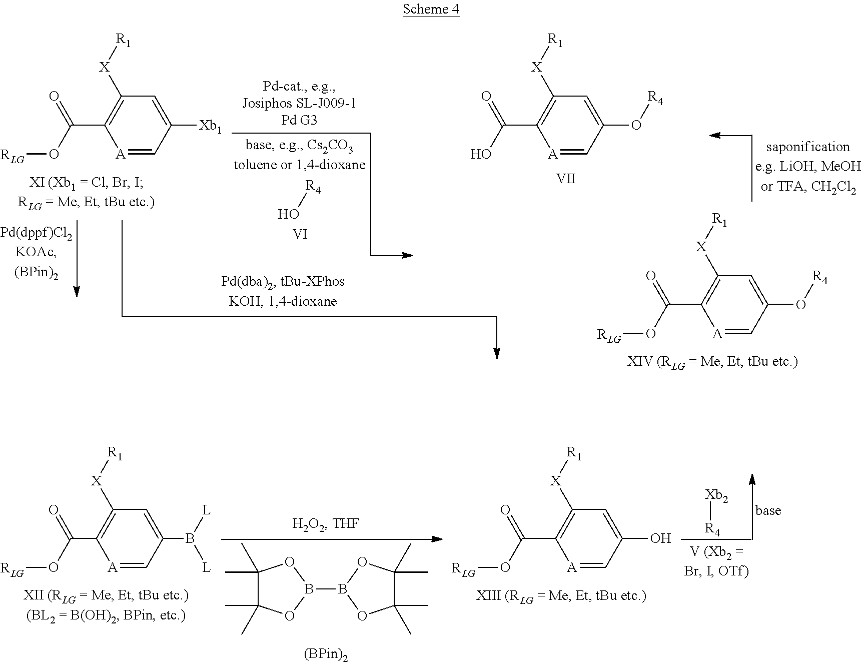

The reactants can be reacted in the presence of a base. Examples of suitable bases are alkali metal or alkaline earth metal hydroxides, alkali metal or alkaline earth metal hydrides, alkali metal or alkaline earth metal amides, alkali metal or alkaline earth metal alkoxides, alkali metal or alkaline earth metal acetates, alkali metal or alkaline earth metal carbonates, alkali metal or alkaline earth metal dialkylamides or alkali metal or alkaline earth metal alkylsilylamides, alkylamines, alkylenediamines, free or N-alkylated saturated or unsaturated cycloalkylamines, basic heterocycles, ammonium hydroxides and carbocyclic amines. Examples which may be mentioned are sodium hydroxide, sodium hydride, sodium amide, sodium methoxide, sodium acetate, sodium carbonate, potassium tert-butoxide, potassium hydroxide, potassium carbonate, potassium hydride, lithium diisopropylamide, potassium bis(trimethylsilyl)amide, calcium hydride, triethylamine, diisopropylethylamine, triethylenediamine, cyclohexylamine, N-cyclohexyl-N,N-dimethylamine, N,N-diethylaniline, pyridine, 4-(N,N-dimethylamino)pyridine, quinuclidine, N-methylmorpholine, benzyltrimethylammonium hydroxide and 1,8-diazabicyclo[5.4.0]undec-7-ene (DBU).

The reactants can be reacted with each other as such, i.e. without adding a solvent or diluent. In most cases, however, it is advantageous to add an inert solvent or diluent or a mixture of these. If the reaction is carried out in the presence of a base, bases which are employed in excess, such as triethylamine, pyridine, N-methylmorpholine or N,N-diethylaniline, may also act as solvents or diluents.

The reaction is advantageously carried out in a temperature range from approximately −80° C. to approximately +140° C., preferably from approximately −30° C. to approximately +100° C., in many cases in the range between ambient temperature and approximately +80° C.

A compound of formula I can be converted in a manner known per se into another compound of formula I by replacing one or more substituents of the starting compound of formula I in the customary manner by (an)other substituent(s) according to the invention.

Depending on the choice of the reaction conditions and starting materials which are suitable in each case, it is possible, for example, in one reaction step only to replace one substituent by another substituent according to the invention, or a plurality of substituents can be replaced by other substituents according to the invention in the same reaction step.

Salts of compounds of formula I can be prepared in a manner known per se. Thus, for example, acid addition salts of compounds of formula I are obtained by treatment with a suitable acid or a suitable ion exchanger reagent and salts with bases are obtained by treatment with a suitable base or with a suitable ion exchanger reagent.

Salts of compounds of formula I can be converted in the customary manner into the free compounds I, acid addition salts, for example, by treatment with a suitable basic compound or with a suitable ion exchanger reagent and salts with bases, for example, by treatment with a suitable acid or with a suitable ion exchanger reagent.

Salts of compounds of formula I can be converted in a manner known per se into other salts of compounds of formula I, acid addition salts, for example, into other acid addition salts, for example by treatment of a salt of inorganic acid such as hydrochloride with a suitable metal salt such as a sodium, barium or silver salt, of an acid, for example with silver acetate, in a suitable solvent in which an inorganic salt which forms, for example silver chloride, is insoluble and thus precipitates from the reaction mixture.

Depending on the procedure or the reaction conditions, the compounds of formula I, which have salt-forming properties can be obtained in free form or in the form of salts.

The compounds of formula I and, where appropriate, the tautomers thereof, in each case in free form or in salt form, can be present in the form of one of the isomers which are possible or as a mixture of these, for example in the form of pure isomers, such as antipodes and/or diastereomers, or as isomer mixtures, such as enantiomer mixtures, for example racemates, diastereomer mixtures or racemate mixtures, depending on the number, absolute and relative configuration of asymmetric carbon atoms which occur in the molecule and/or depending on the configuration of non-aromatic double bonds which occur in the molecule; the invention relates to the pure isomers and also to all isomer mixtures which are possible and is to be understood in each case in this sense hereinabove and hereinbelow, even when stereochemical details are not mentioned specifically in each case.

Diastereomer mixtures or racemate mixtures of compounds of formula I, in free form or in salt form, which can be obtained depending on which starting materials and procedures have been chosen can be separated in a known manner into the pure diasteromers or racemates on the basis of the physicochemical differences of the components, for example by fractional crystallization, distillation and/or chromatography.

Enantiomer mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl cellulose, with the aid of suitable microorganisms, by cleavage with specific, immobilized enzymes, via the formation of inclusion compounds, for example using chiral crown ethers, where only one enantiomer is complexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be set free by the action of suitable agents, for example basic agents.

Pure diastereomers or enantiomers can be obtained according to the invention not only by separating suitable isomer mixtures, but also by generally known methods of diastereoselective or enantioselective synthesis, for example by carrying out the process according to the invention with starting materials of a suitable stereochemistry.

N-oxides can be prepared by reacting a compound of the formula I with a suitable oxidizing agent, for example the H₂O₂/urea adduct in the presence of an acid anhydride, e.g. trifluoroacetic anhydride. Such oxidations are known from the literature, for example from J. Med. Chem., 32 (12), 2561-73, 1989 or WO 00/15615.

It is advantageous to isolate or synthesize in each case the biologically more effective isomer, for example enantiomer or diastereomer, or isomer mixture, for example enantiomer mixture or diastereomer mixture, if the individual components have a different biological activity.

The compounds of formula I and, where appropriate, the tautomers thereof, in each case in free form or in salt form, can, if appropriate, also be obtained in the form of hydrates and/or include other solvents, for example those which may have been used for the crystallization of compounds which are present in solid form.

The compounds of Tables X below can be prepared according to the methods described above. The examples which follow are intended to illustrate the invention and show preferred compounds of formula I.

Table X: This table discloses 60 substituent definitions X.001 to X.060 of the formula I:

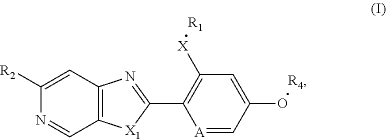

wherein R₂, X₁, A, R₁ and R₄ are as defined below:

TABLE X

| Comp. No | R₂ | X₁ | A | R₁ | R₄ |
|---|---|---|---|---|---|
| X.001 | CF₃ | NMe | N | Et | Me |
| X.002 | CF₃ | NMe | N | Et | Et |
| X.003 | CF₃ | NMe | N | Et | iPr |
| X.004 | CF₃ | NMe | N | Et | tBu |
| X.005 | CF₃ | NMe | N | Et | CH₂cPr |
| X.006 | CF₃ | NMe | N | Et | CH₂CHMe₂ |
| X.007 | CF₃ | NMe | N | Et | CH₂C(O)OMe |
| X.008 | CF₃ | NMe | N | Et | CH(Me)C(O)OMe |
| X.009 | CF₃ | NMe | N | Et | C(Me)2C(O)OMe |
| X.010 | CF₃ | NMe | N | Et | CH₂CN |
| X.011 | CF₃ | NMe | N | Et | CH(Me)CN |
| X.012 | CF₃ | NMe | N | Et | C(Me)₂CN |
| X.013 | CF₃ | NMe | CH | Et | CH₂CH₂OMe |
| X.014 | CF₃ | NMe | N | Et | CH₂CH₂SMe |
| X.015 | CF₃ | NMe | N | Et | CH₂CH₂S(O)Me |
| X.016 | CF₃ | NMe | N | Et | CH₂CH₂S(O)₂Me |
| X.017 | CF₃ | NMe | N | Et | CH₂CHF₂ |
| X.018 | CF₃ | NMe | N | Et | CH₂CF₃ |
| X.019 | CF₃ | NMe | N | Et | CH₂CCH |
| X.020 | CF₃ | NMe | N | Et | C(Me)₂C(O)NH2 |
| X.021 | CF₃ | NMe | N | Et | C(Me)₂C(O)NHMe |
| X.022 | CF₃ | NMe | N | Et | C(Me)₂C(O)NMe₂ |
| X.023 | CF₃ | NMe | N | Et | 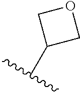 [SMILES: *C1COC1] |
| X.024 | CF₃ | NMe | N | Et | 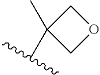 [SMILES: *C1(C)COC1] |
| X.025 | CF₃ | NMe | N | Et | 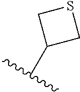 [SMILES: *C1CSC1] |
| X.026 | CF₃ | NMe | N | Et | 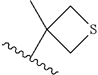 [SMILES: *C1(C)CSC1] |
| X.027 | CF₃ | NMe | N | Et | 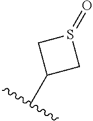 [SMILES: *C1CS(=O)C1] |
| X.028 | CF₃ | NMe | N | Et | 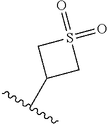 [SMILES: *C1CS(=O)(=O)C1] |
| X.029 | CF₃ | NMe | N | Et | C(Me)₂C(O)NH₂ |
| X.030 | CF₃ | NMe | N | Et | CF₂C(O)NMe₂ |
| X.031 | CF₃ | NMe | N | Et | CH₂SMe |
| X.032 | CF₃ | NMe | N | Et | CH₂S(O)₂Me |
| X.033 | CF₃ | NMe | N | Et | CH₂tBu |
| X.034 | CF₃ | NMe | N | Et | CMe₂C═NOMe |
| X.035 | CF₃ | NMe | N | Et | CH₂C═NOMe |
| X.036 | CF₃ | NMe | N | Et | CHMeC═NOMe |
| X.037 | CF₃ | NMe | N | Et | CMe₂CH₂OMe |
| X.038 | CF₃ | NMe | N | Et | 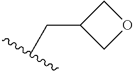 [SMILES: *CC1COC1] |
| X.039 | CF₃ | NMe | N | Et | 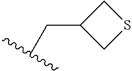 [SMILES: *CC1CSC1] |
| X.040 | CF₃ | NMe | N | Et | 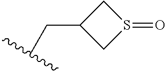 [SMILES: *CC1CS(=O)C1] |
| X.041 | CF₃ | NMe | N | Et | 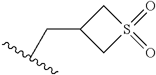 [SMILES: *CC1CS(=O)(=O)C1] |
| X.042 | CF₃ | NMe | N | Et | 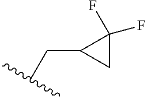 [SMILES: *CC1CC1(F)F] |
| X.043 | CF₃ | NMe | N | Et | CH₂tBu |
| X.044 | CF₃ | NMe | CH | Et | iPr |
| X.045 | CF₃ | NMe | CH | Et | C(Me)₂CN |
| X.046 | CF₃ | NMe | CH | Et | CH₂CHF₂ |
| X.047 | CF₃ | NMe | CH | Et | CH₂CF₃ |
| X.048 | CF₃ | NMe | CH | Et | 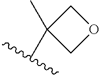 [SMILES: *C1(C)COC1] |
| X.049 | SCF₃ | NMe | N | Et | iPr |
| X.050 | S(O)CF₃ | NMe | N | Et | iPr |
| X.051 | S(O)₂CF₃ | NMe | N | Et | iPr |
| X.052 | SCF₃ | NMe | N | Et | C(Me)₂CN |
| X.053 | S(O)CF₃ | NMe | N | Et | C(Me)₂CN |
| X.054 | S(O)₂CF₃ | NMe | N | Et | C(Me)₂CN |
| X.055 | SCF₃ | NMe | N | Et | CH₂CHF₂ |
| X.056 | S(O)CF₃ | NMe | N | Et | CH₂CHF₂ |
| X.057 | S(O)₂CF₃ | NMe | N | Et | CH₂CHF₂ |
| X.058 | SCF₃ | NMe | N | Et | CH₂CF₃ |
| X.059 | S(O)CF₃ | NMe | N | Et | CH₂CF₃ |
| X.060 | S(O)₂CF₃ | NMe | N | Et | CH₂CF₃ |

and the N-oxides of the compounds of Table X. Me represents the methyl group, Et is the ethyl group, i-Pr is the isopropyl group, C(O)OMe is the methoxycarbonyl group, MeO is the methoxy group, and MeOCH₂ s the methoxymethyl group.

Table 1: This table discloses the 60 compounds 1.001 to 1.060 of the formula I, wherein X is S, and R₂, X₁, A, R₁ and R₄ are as defined in Table X. For example, compound 1.001 has the following structure:

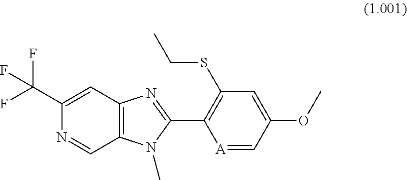

Table 2: This table discloses the 60 compounds 2.001 to 2.060 of the formula I, wherein X is SO, and R₂, X₁, A, R₁ and R₄ are as defined in Table X.

Table 3: This table discloses the 60 compounds 3.001 to 3.060 of the formula I, wherein X is SO₂, and R₂, X₁, A, R₁ and R₄ are as defined in Table X.

The compounds of formula I according to the invention are preventively and/or curatively valuable active ingredients in the field of pest control, even at low rates of application, which have a very favorable biocidal spectrum and are well tolerated by warm-blooded species, fish and plants. The active ingredients according to the invention act against all or individual developmental stages of normally sensitive, but also resistant, animal pests, such as insects or representatives of the order Acarina. The insecticidal or acaricidal activity of the active ingredients according to the invention can manifest itself directly, i. e. in destruction of the pests, which takes place either immediately or only after some time has elapsed, for example during ecdysis, or indirectly, for example in a reduced oviposition and/or hatching rate.

Examples of the abovementioned animal pests are:

from the order Acarina, for example,

Acalitus spp, Aculus spp, Acaricalus spp, Aceria spp, Acarus siro, Amblyomma spp., Argas spp., Boophilus spp., Brevipalpus spp., Bryobia spp, Calipitrimerus spp., Chorioptes spp., Dermanyssus gallinae, Dermatophagoides spp, Eotetranychus spp, Eriophyes spp., Hemitarsonemus spp, Hyalomma spp., Ixodes spp., Olygonychus spp, Ornithodoros spp., Polyphagotarsone latus, Panonychus spp., Phyllocoptruta oleivora, Phytonemus spp, Polyphagotarsonemus spp, Psoroptes spp., Rhipicephalus spp., Rhizoglyphus spp., Sarcoptes spp., Steneotarsonemus spp, Tarsonemus spp. and Tetranychus spp.;

from the order Anoplura, for example,

Haematopinus spp., Linognathus spp., Pediculus spp., Pemphigus spp. and Phylloxera spp.; from the order Coleoptera, for example,

Agriotes spp., Amphimallon majale, Anomala orientalis, Anthonomus spp., Aphodius spp, Astylus atromaculatus, Ataenius spp, Atomaria linearis, Chaetocnema tibialis, Cerotoma spp, Conoderus spp, Cosmopolites spp., Cotinis nitida, Curculio spp., Cyclocephala spp, Dermestes spp., Diabrotica spp., Diloboderus abderus, Epilachna spp., Eremnus spp., Heteronychus arator, Hypothenemus hampei, Lagria vilosa, Leptinotarsa decemLineata, Lissorhoptrus spp., Liogenys spp, Maecolaspis spp, Maladera castanea, Megascelis spp, Melighetes aeneus, Melolontha spp., Myochrous armatus, Orycaephilus spp., Otiorhynchus spp., Phyllophaga spp, Phlyctinus spp., Popillia spp., Psylliodes spp., Rhyssomatus aubtilis, Rhizopertha spp., Scarabeidae, Sitophilus spp., Sitotroga spp., Somaticus spp, Sphenophorus spp, Sternechus subsignatus, Tenebrio spp., Tribolium spp. and Trogoderma spp.;

from the order Diptera, for example,

Aedes spp., Anopheles spp, Antherigona soccata, Bactrocea oleae, Bibio hortulanus, Bradysia spp, Calliphora erythrocephala, Ceratitis spp., Chrysomyia spp., Culex spp., Cuterebra spp., Dacus spp., Delia spp, Drosophila melanogaster, Fannia spp., Gastrophilus spp., Geomyza tripunctata, Glossina spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis spp, Rivelia quadrifasciata, Scatella spp, Sciara spp., Stomoxys spp., Tabanus spp., Tannia spp. and Tipula spp.;

from the order Hemiptera, for example,

Acanthocoris scabrator, Acrosternum spp, Adelphocoris lineolatus, Amblypelta nitida, Bathycoelia thalassina, Blissus spp, Cimex spp., Clavigralla tomentosicollis, Creontiades spp, Distantiella theobroma, Dichelops furcatus, Dysdercus spp., Edessa spp, Euchistus spp., Eurydema pulchrum, Eurygaster spp., Halyomorpha halys, Horcias nobilellus, Leptocorisa spp., Lygus spp, Margarodes spp, Murgantia histrionic, Neomegalotomus spp, Nesidiocoris tenuis, Nezara spp., Nysius simulans, Oebalus insularis, Piesma spp., Piezodorus spp, Rhodnius spp., Sahlbergella singularis, Scaptocoris castanea, Scotinophara spp., Thyanta spp, Triatoma spp., Vatiga illudens;

Acyrthosium pisum, Adalges spp, Agalliana ensigera, Agonoscena targionii, Aleurodicus spp, Aleurocanthus spp, Aleurolobus barodensis, Aleurothrixus floccosus, Aleyrodes brassicae, Amarasca biguttula, Amritodus atkinsoni, Aonidiella spp., Aphididae, Aphis spp., Aspidiotus spp., Aulacorthum solani, Bactericera cockerelli, Bemisia spp, Brachycaudus spp, Brevicoryne brassicae, Cacopsylla spp, Cavariella aegopodii Scop., Ceroplaster spp., Chrysomphalus aonidium, Chrysomphalus dictyospermi, Cicadella spp, Cofana spectra, Cryptomyzus spp, Cicadulina spp, Coccus hesperidum, Dalbulus maidis, Dialeurodes spp, Diaphorina citri, Diuraphis noxia, Dysaphis spp, Empoasca spp., Eriosoma larigerum, Erythroneura spp., Gascardia spp., Glycaspis brimblecombei, Hyadaphis pseudobrassicae, Hyalopterus spp, Hyperomyzus pallidus, Idioscopus clypealis, Jacobiasca lybica, Laodelphax spp., Lecanium corni, Lepidosaphes spp., Lopaphis erysimi, Lyogenys maidis, Macrosiphum spp., Mahanarva spp, Metcalfa pruinosa, Metopolophium dirhodum, Myndus crudus, Myzus spp., Neotoxoptera sp, Nephotettix spp., Nilaparvata spp., Nippolachnus piri Mats, Odonaspis ruthae, Oregma lanigera Zehnter, Parabemisia myricae, Paratrioza cockerelli, Parlatoria spp., Pemphigus spp., Peregrinus maidis, Perkinsiella spp, Phorodon humuli, Phylloxera spp, Planococcus spp., Pseudaulacaspis spp., Pseudococcus spp., Pseudatomoscelis seriatus, Psylla spp., Pulvinaria aethiopica, Quadraspidiotus spp., Quesada gigas, Recilia dorsalis, Rhopalosiphum spp., Saissetia spp., Scaphoideus spp., Schizaphis spp., Sitobion spp., Sogatella furcifera, Spissistilus festinus, Tarophagus Proserpina, Toxoptera spp, Trialeurodes spp, Tridiscus sporoboli, Trionymus spp, Trioza erytreae, Unaspis citri, Zygina flammigera, Zyginidia scutellaris;

from the order Hymenoptera, for example,

Acromyrmex, Arge spp, Atta spp., Cephus spp., Diprion spp., Diprionidae, Gilpinia polytoma, Hoplocamps spp., Lasius spp., Monomorium pharaonis, Neodiprion spp., Pogonomyrmex spp, Slenopsis invicta, Solenopsis spp. and Vespa spp.;

from the order Isoptera, for example,

Coptotermes spp, Corniternes cumulans, Incisitermes spp, Macrotermes spp, Mastotermes spp, Microtermes spp, Reticulitermes spp.; Solenopsis geminate

from the order Lepidoptera, for example,

Acleris spp., Adoxophyes spp., Aegeria spp., Agrotis spp., Alabama argillaceae, Amylois zspp., Anticarsia gemmatalis, Archips spp., Argyresthia spp, Argyrotaenia spp., Autographa spp., Bucculatrix thurberiella, Busseola fusca, Cadra cautella, Carposina nipponensis, Chilo spp., Choristoneura spp., Chrysoteuchia topiaria, Clysia ambiguella, Cnaphalocrocis spp., Cnephasia spp., Cochylis spp., Coleophora spp., Colias lesbia, Cosmophila flava, Crambus spp, Crocidolomia binotalis, Cryptophlebia leucotreta, Cydalima perspectalis, Cydia spp., Diaphania perspectalis, Diatraea spp., Diparopsis castanea, Earias spp., Eldana saccharina, Ephestia spp., Epinotia spp, Estigmene acrea, Etiella zinckinella, Eucosma spp., Eupoecilia ambiguella, Euproctis spp., Euxoa spp., Feltia jaculiferia, Grapholita spp., Hedya nubiferana, Heliothis spp., Hellula undalis, Herpetogramma spp, Hyphantria cunea, Keiferia lycopersicella, Lasmopalpus lignosellus, Leucoptera scitella, Lithocollethis spp., Lobesia botrana, Loxostege bifidalis, Lymantria spp., Lyonetia spp., Malacosoma spp., Mamestra brassicae, Manduca sexta, Mythimna spp, Noctua spp, Operophtera spp., Orniodes indica, Ostrinia nubilalis, Pammene spp., Pandemis spp., Panolis flammea, Papaipema nebris, Pectinophora gossypiela, Perileucoptera coffeella, Pseudaletia unipuncta, Phthorimaea operculella, Pieris rapae, Pieris spp., Plutella xylostella, Prays spp., Pseudoplusia spp, Rachiplusia nu, Richia albicosta, Scirpophaga spp., Sesamia spp., Sparganothis spp., Spodoptera spp., Sylepta derogate, Synanthedon spp., Thaumetopoea spp., Tortrix spp., Trichoplusia ni, Tuta absoluta, and Yponomeuta spp.;

from the order Mallophaga, for example,

Damalinea spp. and Trichodectes spp.;

from the order Orthoptera, for example,

Blatta spp., Blattella spp., Gryllotalpa spp., Leucophaea maderae, Locusta spp., Neocurtilla hexadactyla, Periplaneta spp., Scapteriscus spp, and Schistocerca spp.;

from the order Psocoptera, for example,

Liposcelis spp.;

from the order Siphonaptera, for example,

Ceratophyllus spp., Ctenocephalides spp. and Xenopsylla cheopis;

from the order Thysanoptera, for example,

Calliothrips phaseoli, Frankliniella spp., Heliothrips spp, Hercinothrips spp., Parthenothrips spp, Scirtothrips aurantii, Sericothrips variabilis, Taeniothrips spp., Thrips spp;

from the order Thysanura, for example, Lepisma saccharina.

The active ingredients according to the invention can be used for controlling, i. e. containing or destroying, pests of the abovementioned type which occur in particular on plants, especially on useful plants and ornamentals in agriculture, in horticulture and in forests, or on organs, such as fruits, flowers, foliage, stalks, tubers or roots, of such plants, and in some cases even plant organs which are formed at a later point in time remain protected against these pests.

Suitable target crops are, in particular, cereals, such as wheat, barley, rye, oats, rice, maize or sorghum; beet, such as sugar or fodder beet; fruit, for example pomaceous fruit, stone fruit or soft fruit, such as apples, pears, plums, peaches, almonds, cherries or berries, for example strawberries, raspberries or blackberries; leguminous crops, such as beans, lentils, peas or soya; oil crops, such as oilseed rape, mustard, poppies, olives, sunflowers, coconut, castor, cocoa or ground nuts; cucurbits, such as pumpkins, cucumbers or melons; fibre plants, such as cotton, flax, hemp or jute; citrus fruit, such as oranges, lemons, grapefruit or tangerines; vegetables, such as spinach, lettuce, asparagus, cabbages, carrots, onions, tomatoes, potatoes or bell peppers; Lauraceae, such as avocado, Cinnamonium or camphor; and also tobacco, nuts, coffee, eggplants, sugarcane, tea, pepper, grapevines, hops, the plantain family and latex plants.

The compositions and/or methods of the present invention may be also used on any ornamental and/or vegetable crops, including flowers, shrubs, broad-leaved trees and evergreens. For example the invention may be used on any of the following ornamental species: Ageratum spp., Alonsoa spp., Anemone spp., Anisodontea capsenisis, Anthemis spp., Antirrhinum spp., Aster spp., Begonia spp. (e.g. B. elatior, B. semperflorens, B. tubéreux), Bougainvillea spp., Brachycome spp., Brassica spp. (ornamental), Calceolaria spp., Capsicum annuum, Catharanthus roseus, Canna spp., Centaurea spp., Chrysanthemum spp., Cineraria spp. (C. maritime), Coreopsis spp., Crassula coccinea, Cuphea ignea, Dahlia spp., Delphinium spp., Dicentra spectabilis, Dorotheantus spp., Eustoma grandiflorum, Forsythia spp., Fuchsia spp., Geranium gnaphalium, Gerbera spp., Gomphrena globosa, Heliotropium spp., Helianthus spp., Hibiscus spp., Hortensia spp., Hydrangea spp., Hypoestes phyllostachya, Impatiens spp. (I. Walleriana), Iresines spp., Kalanchoe spp., Lantana camara, Lavatera trimestris, Leonotis leonurus, Lilium spp., Mesembryanthemum spp., Mimulus spp., Monarda spp., Nemesia spp., Tagetes spp., Dianthus spp. (carnation), Canna spp., Oxalis spp., Bellis spp., Pelargonium spp. (P. peltatum, P. Zonale), Viola spp. (pansy), Petunia spp., Phlox spp., Plecthranthus spp., Poinsettia spp., Parthenocissus spp. (P. quinquefolia, P. tricuspidata), Primula spp., Ranunculus spp., Rhododendron spp., Rosa spp. (rose), Rudbeckia spp., Saintpaulia spp., Salvia spp., Scaevola aemola, Schizanthus wisetonensis, Sedum spp., Solanum spp., Surfinia spp., Tagetes spp., Nicotinia spp., Verbena spp., Zinnia spp. and other bedding plants.

For example the invention may be used on any of the following vegetable species: Allium spp. (A. sativum, A. cepa, A. oschaninii, A. Porrum, A. ascalonicum, A. fistulosum), Anthriscus cerefolium, Apium graveolus, Asparagus officinalis, Beta vulgarus, Brassica spp. (B. Oleracea, B. Pekinensis, B. rapa), Capsicum annuum, Cicer arietinum, Cichorium endivia, Cichorum spp. (C. intybus, C. endivia), Citrillus lanatus, Cucumis spp. (C. sativus, C. melo), Cucurbita spp. (C. pepo, C. maxima), Cyanara spp. (C. scolymus, C. cardunculus), Daucus carota, Foeniculum vulgare, Hypericum spp., Lactuca sativa, Lycopersicon spp. (L. esculentum, L. lycopersicum), Mentha spp., Ocimum basilicum, Petroselinum crispum, Phaseolus spp. (P. vulgaris, P. coccineus), Pisum sativum, Raphanus sativus, Rheum rhaponticum, Rosemarinus spp., Salvia spp., Scorzonera hispanica, Solanum melongena, Spinacea oleracea, Valerianella spp. (V. locusta, V. eriocarpa) and Vicia faba.

Preferred ornamental species include African violet, Begonia, Dahlia, Gerbera, Hydrangea, Verbena, Rosa, Kalanchoe, Poinsettia, Aster, Centaurea, Coreopsis, Delphinium, Monarda, Phlox, Rudbeckia, Sedum, Petunia, Viola, Impatiens, Geranium, Chrysanthemum, Ranunculus, Fuchsia, Salvia, Hortensia, rosemary, sage, St. Johnswort, mint, sweet pepper, tomato and cucumber.

The active ingredients according to the invention are especially suitable for controlling Aphis craccivora, Diabrotica balteata, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca (preferably in vegetables, vineyards), Leptinotarsa (preferably in potatos) and Chilo supressalis (preferably in rice).

The active ingredients according to the invention are especially suitable for controlling Aphis craccivora, Diabrotica balteata, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca (preferably in vegetables, vineyards), Leptinotarsa (preferably in potatos) and Chilo supressalis (preferably in rice).

In a further aspect, the invention may also relate to a method of controlling damage to plant and parts thereof by plant parasitic nematodes (Endoparasitic-, Semiendoparasitic- and Ectoparasitic nematodes), especially plant parasitic nematodes such as root knot nematodes, Meloidogyne hapla, Meloidogyne incognita, Meloidogyne javanica, Meloidogyne arenaria and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Pratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species, such as Subanguina spp., Hypsoperine spp., Macroposthonia spp., Melinius spp., Punctodera spp., and Quinisulcius spp.

The compounds of the invention may also have activity against the molluscs. Examples of which include, for example, Ampullariidae; Arion (A. ater, A. circumscriptus, A. hortensis, A. rufus); Bradybaenidae (Bradybaena fruticum); Cepaea (C. hortensis, C. Nemoralis); ochlodina; Deroceras (D. agrestis, D. empiricorum, D. laeve, D. reticulatum); Discus (D. rotundatus); Euomphalia; Galba (G. trunculata); Helicelia (H. itala, H. obvia); Helicidae Helicigona arbustorum); Helicodiscus; Helix (H. aperta); Limax (L. cinereoniger, L. flavus, L. marginatus, L. maximus, L. tenellus); Lymnaea; Milax (M. gagates, M. marginatus, M. sowerbyi); Opeas; Pomacea (P. canaticulata); Vallonia and Zanitoides.

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins, for example insecticidal proteins from Bacillus cereus or Bacillus popilliae; or insecticidal proteins from Bacillus thuringiensis, such as δ-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

In the context of the present invention there are to be understood by δ-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810). Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.

The processes for the preparation of such transgenic plants are generally known to the person skilled in the art and are described, for example, in the publications mentioned above. CryI-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and moths (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:

1. Bt11 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.

2. Bt176 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.

3. MIR604 Maize from Syngenta Seeds SAS, Chemin de l'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.

4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.

5. IPC 531 Cotton from Monsanto Europe S.A. 270-272 Avenue de Tervuren, B-1150 Brussels, Belgium, registration number C/ES/96/02.

6. 1507 Maize from Pioneer Overseas Corporation, Avenue Tedesco, 7 B-1160 Brussels, Belgium, registration number C/NL/00/10. Genetically modified maize for the expression of the protein CrylF for achieving resistance to certain Lepidoptera insects and of the PAT protein for achieving tolerance to the herbicide glufosinate ammonium.

7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

Transgenic crops of insect-resistant plants are also described in BATS (Zentrum für Biosicherheit and Nachhaltigkeit, Zentrum BATS, Clarastrasse 13, 4058 Basel, Switzerland) Report 2003, (http://bats.ch).

The term “crops” is to be understood as including also crop plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818 and EP-A-0 353 191. The methods of producing such transgenic plants are generally known to the person skilled in the art and are described, for example, in the publications mentioned above.

Crops may also be modified for enhanced resistance to fungal (for example Fusarium, Anthracnose, or Phytophthora), bacterial (for example Pseudomonas) or viral (for example potato leafroll virus, tomato spotted wilt virus, cucumber mosaic virus) pathogens.

Crops also include those that have enhanced resistance to nematodes, such as the soybean cyst nematode.

Crops that are tolerance to abiotic stress include those that have enhanced tolerance to drought, high salt, high temperature, chill, frost, or light radiation, for example through expression of NF-YB or other proteins known in the art.

Antipathogenic substances which can be expressed by such transgenic plants include, for example, ion channel blockers, such as blockers for sodium and calcium channels, for example the viral KP1, KP4 or KP6 toxins; stilbene synthases; bibenzyl synthases; chitinases; glucanases; the so-called “pathogenesis-related proteins” (PRPs; see e.g. EP-A-0 392 225); antipathogenic substances produced by microorganisms, for example peptide antibiotics or heterocyclic antibiotics (see e.g. WO 95/33818) or protein or polypeptide factors involved in plant pathogen defence (so-called “plant disease resistance genes”, as described in WO 03/000906).

Further areas of use of the compositions according to the invention are the protection of stored goods and store rooms and the protection of raw materials, such as wood, textiles, floor coverings or buildings, and also in the hygiene sector, especially the protection of humans, domestic animals and productive livestock against pests of the mentioned type.

The present invention also provides a method for controlling pests (such as mosquitoes and other disease vectors; see also http://www.who.int/malaria/vector_control/irs/en/). In one embodiment, the method for controlling pests comprises applying the compositions of the invention to the target pests, to their locus or to a surface or substrate by brushing, rolling, spraying, spreading or dipping. By way of example, an IRS (indoor residual spraying) application of a surface such as a wall, ceiling or floor surface is contemplated by the method of the invention. In another embodiment, it is contemplated to apply such compositions to a substrate such as non-woven or a fabric material in the form of (or which can be used in the manufacture of) netting, clothing, bedding, curtains and tents.

In one embodiment, the method for controlling such pests comprises applying a pesticidally effective amount of the compositions of the invention to the target pests, to their locus, or to a surface or substrate so as to provide effective residual pesticidal activity on the surface or substrate. Such application may be made by brushing, rolling, spraying, spreading or dipping the pesticidal composition of the invention. By way of example, an IRS application of a surface such as a wall, ceiling or floor surface is contemplated by the method of the invention so as to provide effective residual pesticidal activity on the surface. In another embodiment, it is contemplated to apply such compositions for residual control of pests on a substrate such as a fabric material in the form of (or which can be used in the manufacture of) netting, clothing, bedding, curtains and tents.

Substrates including non-woven, fabrics or netting to be treated may be made of natural fibres such as cotton, raffia, jute, flax, sisal, hessian, or wool, or synthetic fibres such as polyamide, polyester, polypropylene, polyacrylonitrile or the like. The polyesters are particularly suitable. The methods of textile treatment are known, e.g. WO 2008/151984, WO 2003/034823, U.S. Pat. No. 5,631,072, WO 2005/64072, WO2006/128870, EP 1724392, WO 2005113886 or WO 2007/090739.

Further areas of use of the compositions according to the invention are the field of tree injection/trunk treatment for all ornamental trees as well all sort of fruit and nut trees.

In the field of tree injection/trunk treatment, the compounds according to the present invention are especially suitable against wood-boring insects from the order Lepidoptera as mentioned above and from the order Coleoptera, especially against woodborers listed in the following tables A and B:

TABLE A
Examples of exotic woodborers of economic importance.

| Family | Species | Host or Crop Infested |
|---|---|---|
| Buprestidae | Agrilus planipennis | Ash |
| Cerambycidae | Anoplura glabripennis | Hardwoods |
| Scolytidae | Xylosandrus crassiusculus | Hardwoods |
| | X. mutilatus | Hardwoods |
| | Tomicus piniperda | Conifers |

TABLE B
Examples of native woodborers of economic importance.

| Family | Species | Host or Crop Infested |
|---|---|---|
| Buprestidae | Agrilus anxius | Birch |
| | Agrilus politus | Willow, Maple |
| | Agrilus sayi | Bayberry, Sweetfern |
| | Agrilus vittaticoillis | Apple, Pear, Cranberry, |
| | | Serviceberry, Hawthorn |
| | Chrysobothris femorata | Apple, Apricot, Beech, Boxelder, |
| | | Cherry, Chestnut, Currant, Elm, |
| | | Hawthorn, Hackberry, Hickory, |
| | | Horsechestnut, Linden, Maple, |
| | | Mountain-ash, Oak, Pecan, Pear, |
| | | Peach, Persimmon, Plum, Poplar, |
| | | Quince, Redbud, Serviceberry, |
| | | Sycamore, Walnut, Willow |
| | Texania campestris | Basswood, Beech, Maple, Oak, |
| | | Sycamore, Willow, Yellow-poplar |
| Cerambycidae | Goes pulverulentus | Beech, Elm, Nuttall, Willow, Black |
| | | oak, Cherrybark oak, Water oak, |
| | | Sycamore |
| | Goes tigrinus | Oak |
| | Neoclytus acuminatus | Ash, Hickory, Oak, Walnut, Birch, |
| | | Beech, Maple, Eastern |
| | | hophornbeam, Dogwood, |
| | | Persimmon, Redbud, Holly, |
| | | Hackberry, Black locust, |
| | | Honeylocust, Yellow-poplar, |
| | | Chestnut, Osage-orange, Sassafras, |
| | | Lilac, Mountain-mahogany, Pear, |
| | | Cherry, Plum, Peach, Apple, Elm, |
| | | Basswood, Sweetgum |
| | Neoptychodes trilineatus | Fig, Alder, Mulberry, Willow, Netleaf |
| | | hackberry |
| | Oberea ocellata | Sumac, Apple, Peach, Plum, Pear, |
| | | Currant, Blackberry |
| | Oberea tripunctata | Dogwood, Viburnum, Elm, |
| | | Sourwood, Blueberry, |
| | | Rhododendron, Azalea, Laurel, |
| | | Poplar, Willow, Mulberry |
| | Oncideres cingulata | Hickory, Pecan, Persimmon, Elm, |
| | | Sourwood, Basswood, Honeylocust, |
| | | Dogwood, Eucalyptus, Oak, |
| | | Hackberry, Maple, Fruit trees |
| | Saperda calcarata | Poplar |
| | Strophiona nitens | Chestnut, Oak, Hickory, Walnut, |
| | | Beech, Maple |
| Scolytidae | Corthylus columbianus | Maple, Oak, Yellow-poplar, Beech, |
| | | Boxelder, Sycamore, Birch, |
| | | Basswood, Chestnut, Elm |
| | Dendroctonus frontalis | Pine |
| | Dryocoetes betulae | Birch, Sweetgum, Wild cherry, |
| | | Beech, Pear |
| | Monarthrum fasciatum | Oak, Maple, Birch, Chestnut, |
| | | Sweetgum, Blackgum, Poplar, |
| | | Hickory, Mimosa, Apple, Peach, Pine |
| | Phloeotribus liminaris | Peach, Cherry, Plum, Black cherry, |
| | | Elm, Mulberry, Mountain-ash |
| | Pseudopityophthorus pruinosus | Oak, American beech, Black cherry, |
| | | Chickasaw plum, Chestnut, Maple, |
| | | Hickory, Hornbeam, Hophornbeam |
| Sesiidae | Paranthrene simulans | Oak, American chestnut |
| | Sannina uroceriformis | Persimmon |
| | Synanthedon exitiosa | Peach, Plum, Nectarine, Cherry, |
| | | Apricot, Almond, Black cherry |
| | Synanthedon pictipes | Peach, Plum, Cherry, Beach, Black |
| | | Cherry |
| | Synanthedon rubrofascia | Tupelo |
| | Synanthedon scitula | Dogwood, Pecan, Hickory, Oak, |
| | | Chestnut, Beech, Birch, Black cherry, |
| | | Elm, Mountain-ash, Viburnum, |
| | | Willow, Apple, Loquat, Ninebark, |
| | | Bayberry |
| | Vitacea polistiformis | Grape |

The present invention may be also used to control any insect pests that may be present in turfgrass, including for example beetles, caterpillars, fire ants, ground pearls, millipedes, sow bugs, mites, mole crickets, scales, mealybugs ticks, spittlebugs, southern chinch bugs and white grubs. The present invention may be used to control insect pests at various stages of their life cycle, including eggs, larvae, nymphs and adults.

In particular, the present invention may be used to control insect pests that feed on the roots of turfgrass including white grubs (such as Cyclocephala spp. (e.g. masked chafer, C. lurida), Rhizotrogus spp. (e.g. European chafer, R. majalis), Cotinus spp. (e.g. Green June beetle, C. nitida), Popillia spp. (e.g. Japanese beetle, P. japonica), Phyllophaga spp. (e.g. May/June beetle), Ataenius spp. (e.g. Black turfgrass ataenius, A. spretulus), Maladera spp. (e.g. Asiatic garden beetle, M. castanea) and Tomarus spp.), ground pearls (Margarodes spp.), mole crickets (tawny, southern, and short-winged; Scapteriscus spp., Gryllotalpa africana) and leatherjackets (European crane fly, Tipula spp.).

The present invention may also be used to control insect pests of turfgrass that are thatch dwelling, including armyworms (such as fall armyworm Spodoptera frugiperda, and common armyworm Pseudaletia unipuncta), cutworms, billbugs (Sphenophorus spp., such as S. venatus verstitus and S. parvulus), and sod webworms (such as Crambus spp. and the tropical sod webworm, Herpetogramma phaeopteralis).

The present invention may also be used to control insect pests of turfgrass that live above the ground and feed on the turfgrass leaves, including chinch bugs (such as southern chinch bugs, Blissus insularis), Bermudagrass mite (Eriophyes cynodoniensis), rhodesgrass mealybug (Antonina graminis), two-lined spittlebug (Propsapia bicincta), leafhoppers, cutworms (Noctuidae family), and greenbugs. The present invention may also be used to control other pests of turfgrass such as red imported fire ants (Solenopsis invicta) that create ant mounds in turf.

In the hygiene sector, the compositions according to the invention are active against ectoparasites such as hard ticks, soft ticks, mange mites, harvest mites, flies (biting and licking), parasitic fly larvae, lice, hair lice, bird lice and fleas.

Examples of such parasites are:

Of the order Anoplurida: Haematopinus spp., Linognathus spp., Pediculus spp. and Phtirus spp., Solenopotes spp.

Of the order Mallophagida: Trimenopon spp., Menopon spp., Trinoton spp., Bovicola spp., Werneckiella spp., Lepikentron spp., Damalina spp., Trichodectes spp. and Felicola spp.

Of the order Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

Of the order Siphonapterida, for example Pulex spp., Ctenocephalides spp., Xenopsylla spp., Ceratophyllus spp.

Of the order Heteropterida, for example Cimex spp., Triatoma spp., Rhodnius spp., Panstrongylus Spp.

Of the order Blattarida, for example Blatta orientalis, Periplaneta americana, Blattelagermanica and Supella spp.

Of the subclass Acaria (Acarida) and the orders Meta- and Meso-stigmata, for example Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.

Of the orders Actinedida (Prostigmata) and Acaridida (Astigmata), for example Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The compositions according to the invention are also suitable for protecting against insect infestation in the case of materials such as wood, textiles, plastics, adhesives, glues, paints, paper and card, leather, floor coverings and buildings.

The compositions according to the invention can be used, for example, against the following pests: beetles such as Hylotrupes bajulus, Chlorophorus pilosis, Anobium punctatum, Xestobium rufovillosum, Ptilinuspecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthesrugicollis, Xyleborus spec., Tryptodendron spec., Apate monachus, Bostrychus capucins, Heterobostrychus brunneus, Sinoxylon spec. and Dinoderus minutus, and also hymenopterans such as Sirex juvencus, Urocerus gigas, Urocerus gigas taignus and Urocerus augur, and termites such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis and Coptotermes formosanus, and bristletails such as Lepisma saccharina.

The compounds according to the invention can be used as pesticidal agents in unmodified form, but they are generally formulated into compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances. A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in McCutcheon's Detergents and Emulsifiers Annual, MC Publishing Corp., Ridgewood New Jersey (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers. The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10th Edition, Southern Illinois University, 2010.

The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 l/ha, especially from 10 to 1000 l/ha.

Preferred formulations can have the following compositions (weight %):

Emulsifiable Concentrates:

active ingredient: 1 to 95%, preferably 60 to 90%

surface-active agent: 1 to 30%, preferably 5 to 20%

liquid carrier: 1 to 80%, preferably 1 to 35%

Dusts:

active ingredient: 0.1 to 10%, preferably 0.1 to 5%

solid carrier: 99.9 to 90%, preferably 99.9 to 99%

Suspension Concentrates:

active ingredient: 5 to 75%, preferably 10 to 50%

water: 94 to 24%, preferably 88 to 30%

surface-active agent: 1 to 40%, preferably 2 to 30%

Wettable Powders:

active ingredient: 0.5 to 90%, preferably 1 to 80%

surface-active agent: 0.5 to 20%, preferably 1 to 15%

solid carrier: 5 to 95%, preferably 15 to 90%

Granules:

active ingredient: 0.1 to 30%, preferably 0.1 to 15%

solid carrier: 99.5 to 70%, preferably 97 to 85%

The following Examples further illustrate, but do not limit, the invention.

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredients | 25% | 50% | 75% |
| light mineral oil | 5% | 5% | 5% |
| highly dispersed silicic acid | 5% | 5% | — |
| Kaolin | 65% | 40% | — |
| Talcum | — | | 20% |

The combination is thoroughly mixed with the adjuvants and the mixture is thoroughly ground in a suitable mill, affording powders that can be used directly for seed treatment.

| Emulsifiable concentrate | |
|---|---|
| active ingredients | 10% |
| octylphenol polyethylene glycol ether | 3% |
| (4-5 mol of ethylene oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill. Such powders can also be used for dry dressings for seed.

| Extruder granules | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

| Coated granules | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

Suspension Concentrate

| | |
|---|---|
| active ingredients | 40% |
| propylene glycol | 10% |
| nonylphenol polyethylene glycol ether | 6% |
| (15 mol of ethylene oxide) | |
| Sodium lignosulfonate | 10% |
| carboxymethylcellulose | 1% |
| silicone oil (in the form of a 75% emulsion in water) | 1% |
| Water | 32% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Flowable Concentrate for Seed Treatment

| | |
|---|---|
| active ingredients | 40% |
| propylene glycol | 5% |
| copolymer butanol PO/EO | 2% |
| Tristyrenephenole with 10-20 moles EO | 2% |
| 1,2-benzisothiazolin-3-one (in the form of a | 0.5% |
| 20% solution in water) | |
| monoazo-pigment calcium salt | 5% |
| Silicone oil (in the form of a 75% emulsion in water) | 0.2% |
| Water | 45.3% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed. The obtained capsule suspension is stabilized by adding 0.25 parts of a thickener and 3 parts of a dispersing agent. The capsule suspension formulation contains 28% of the active ingredients. The medium capsule diameter is 8-15 microns. The resulting formulation is applied to seeds as an aqueous suspension in an apparatus suitable for that purpose.

Formulation types include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP), a soluble granule (SG) or any technically feasible formulation in combination with agriculturally acceptable adjuvants.

### PREPARATORY EXAMPLES

“Mp” means melting point in ° C. Free radicals represent methyl groups. ¹H NMR measurements were recorded on a Brucker 400 MHz spectrometer, chemical shifts are given in ppm relevant to a TMS standard. Spectra measured in deuterated solvents as indicated. Either one of the LCMS methods below was used to characterize the compounds. The characteristic LCMS values obtained for each compound were the retention time (“Rt”, recorded in minutes) and the measured molecular ion (M+H)⁺ or (M−H)⁻.

LCMS Methods:

Method 1: Standard 1

Spectra were recorded on a Mass Spectrometer from Waters (SQD, SQDII Single quadrupole mass spectrometer) equipped with an electrospray source (Polarity: positive and negative ions, Capillary: 3.00 kV, Cone range: 30 V, Extractor: 2.00 V, Source Temperature: 150° C., Desolvation Temperature: 350° C., Cone Gas Flow: 50 l/h, Desolvation Gas Flow: 650 l/h, Mass range: 100 to 900 Da) and an Acquity UPLC from Waters: Binary pump, heated column compartment, diode-array detector and ELSD detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH, gradient: 10-100% B in 1.2 min; Flow (ml/min) 0.85

Method 2: Standard Long

Spectra were recorded on a Mass Spectrometer from Waters (SQD, SODII Single quadrupole mass spectrometer) equipped with an electrospray source (Polarity: positive and negative ions), Capillary: 3.00 kV, Cone range: 30V, Extractor: 2.00 V, Source Temperature: 150° C., Desolvation Temperature: 350° C., Cone Gas Flow: 50 l/h, Desolvation Gas Flow: 650 l/h, Mass range: 100 to 900 Da) and an Acquity UPLC from Waters: Binary pump, heated column compartment, diode-array detector and ELSD detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH, gradient: 10-100% B in 2.7 min; Flow (ml/min) 0.85

Method 3: Standard 2 Long

Spectra were recorded on a Mass Spectrometer from Agilent Technologies (6410 Triple Quadrupole mass spectrometer) equipped with an equipped with an electrospray source (Polarity: positive or negative ions, MS2 Scan, Capillary: 4.00 kV, Fragmentor: 100 V, Desolvatation Temperature: 350° C., Gas Flow: 11 L/min, Nebulizer Gas: 45 psi, Mass range: 110 to 1000 Da) and a 1200 Series HPLC from Agilent: pump, heated column compartment and diode-array detector. Column: KINETEX EVO C18, 2.6 μm, 50×4.6 mm, Temp: 40° C., DAD Wavelength range (nm): 210 to 400, Solvent Gradient: A=water+5% Acetonitrile+0.1% HCOOH, B=Acetonitrile+0.1% HCOOH: gradient: 0 min 0% B, 100% A; 0.9-1.8 min 100% B; Flow (ml/min) 1.8.

Method 4: Standard 2

Spectra were recorded on a Mass Spectrometer from Waters equipped with an electrospray source (Polarity: positive or negative ions, Full Scan, Capillary: 3.00 kV, Cone range: 41 V, Source Temperature: 150° C., Desolvation Temperature: 500° C., Cone Gas Flow: 50 L/Hr, Desolvation Gas Flow: 1000 L/Hr, Mass range: 110 to 800 Da) and a H-Class UPLC from Waters: Binary pump, heated column compartment and diode-array detector. Column: Waters UPLC HSS T3 C18, 1.8 μm, 30×2.1 mm, Temp: 40° C., DAD Wavelength range (nm): 210 to 400, Solvent Gradient: A=water+5% Acetonitrile+0.1% HCOOH, B=Acetonitrile+0.1% HCOOH: gradient: 0 min 10% B; 0.-0.2 min 10-50% B; 0.2-0.7 min 50-100% B; Flow (ml/min) 0.8.

### Example E1: Preparation of 2-[3-ethylsulfonyl-5-(oxetan-3-yloxy)-2-pyridyl]-3-methyl-6-(trifluoromethyl)imidazo[4,5-c]pyridine (Compound P1)

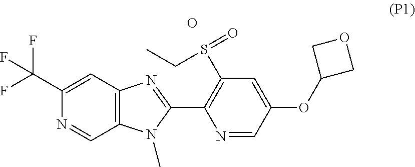

### Step 1: Preparation of 2-[3-ethylsulfanyl-5-(oxetan-3-yloxy)-2-pyridyl]-3-methyl-6-(trifluoromethyl)imidazo[4,5-c]pyridine (Compound P2)

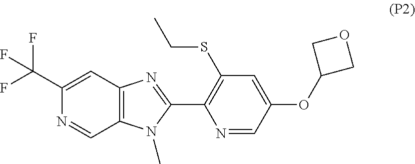 [SMILES: CCSc1cc(OC2COC2)cnc1-c1nc2cc(C(F)(F)F)ncc2n1C]

A microwave vial was charged with 2-(5-bromo-3-ethylsulfanyl-2-pyridyl)-3-methyl-6-(trifluoromethyl)imidazo[4,5-c]pyridine (prepared according to WO16096584) (1.00 g, 2.40 mmol), toluene (12.0 ml), cesium carbonate (1.57 g, 4.80 mmol, 2.00 equiv.) and oxetan-3-ol (280 mg, 240 μI, 3.60 mmol, 1.50 equiv.). After degassing with nitrogen for 20 min, Josiphos SL-J009-1 Pd G3 (111 mg, 0.12 mmol, 0.05 equiv) was added. The vessel was sealed and heated in an oil bath at 120° C. for 2 hours. After cooling to room temperature, the reaction mixture was poured into water, and the aqueous phase was extracted twice with ethyl acetate. The combined organic phases were washed with brine, dried over sodium sulfate, filtered and concentrated under vaccum. The crude material was purified by column chromatography over silica gel, eluting with ethyl acetate in hexanes. The selected fractions were evaporated to yield the title compound as a solid. LCMS (method 4): 411 (M+H)⁺; retention time: 0.95 min.

### Step 2: Preparation of 2-[3-ethylsulfonyl-5-(oxetan-3-yloxy)-2-pyridyl]-3-methyl-6-(trifluoromethyl)imidazo[4,5-c]pyridine (Compound P1)

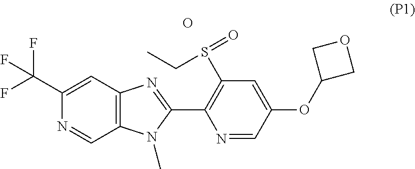

To a solution of 2-[3-ethylsulfanyl-5-(oxetan-3-yloxy)-2-pyridyl]-3-methyl-6-(trifluoromethyl)imidazo[4,5-c]pyridine (120 mg, 0.29 mmol) in dichloromethane (2.9 ml) under argon atmosphere at 0° C. was added 3-chloroperbenzoic acid (160 mg, 0.70 mmol, 2.4 equiv.). After stirring for 15 min at 0° C. then 2 hours at room temperature, the reaction mixture was poured into water, and the aqueous phase was extracted three times with dichloromethane. The combined organic phases were washed with a saturated aqueous sodium bicarbonate solution, dried over sodium sulfate, filtered and concentrated. The crude material was purified by column chromatography over silica gel, eluting with ethyl acetate in hexanes to give the title compound as a solid. LCMS (method 4): 443 (M+H)⁺; retention time: 1.34 min.

### Example E2: Preparation of 2-[3-ethylsulfonyl-5-(3-methyloxetan-3-yl)oxy-2-pyridyl]-3-methyl-6-(trifluoromethyl)imidazo[4,5-c]pyridine (Compound P4)

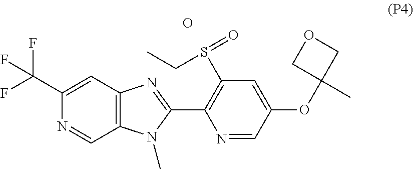

### Step 1: Preparation of 2-[3-ethylsulfanyl-5-(3-methyloxetan-3-yl)oxy-2-pyridyl]-3-methyl-6-(trifluoromethyl)imidazo[4,5-c]pyridine (Compound P14)

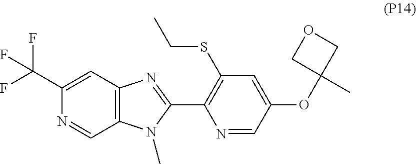 [SMILES: CCSc1cc(OC2(C)COC2)cnc1-c1nc2cc(C(F)(F)F)ncc2n1C]

A microwave vial was charged with 2-(5-bromo-3-ethylsulfanyl-2-pyridyl)-3-methyl-6-(trifluoromethyl)imidazo[4,5-c]pyridine (prepared according to WO16096584) (500 mg, 1.20 mmol), toluene (6.0 ml), cesium carbonate (786 mg, 2.40 mmol, 2.00 equiv.) and Josiphos SL-J009-1 Pd G3 (55 mg, 0.06 mmol, 0.05 equiv). After degassing with argon for 5 min, 3-methyl-oxetan-3-ol (158 mg, 140 μl, 1.80 mmol, 1.50 equiv.) was added. The vessel was sealed and heated in an oil bath at 130° C. overnight. After cooling to room temperature, the reaction mixture was poured into water, and the aqueous phase was extracted twice with ethyl acetate. The combined organic phases were washed with brine, dried over sodium sulfate, filtered and concentrated under vacuum. Purification over silica gel eluting with ethyl acetate in dichloromethane afforded the title compound as a yellow solid. LCMS (method 1): 425 (M+H)⁺; retention time: 0.95 min.

### Step 2: Preparation of 2-[3-ethylsulfonyl-5-(3-methyloxetan-3-yl)oxy-2-pyridyl]-3-methyl-6-(trifluoromethyl)imidazo[4,5-c]pyridine (Compound P4)

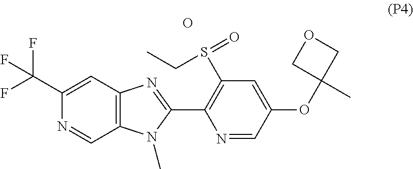

According to the same procedure described above in step 2 for example E1, a mixture of 2-[3-ethylsulfanyl-5-(3-methyloxetan-3-yl)oxy-2-pyridyl]-3-methyl-6-(trifluoromethyl)imidazo[4,5-c]pyridine (250 mg, 0.59 mmol) and 3-chloroperbenzoic acid (224 mg, 1.30 mmol, 2.2 equiv.) in dichloromethane (4.7 ml) under argon atmosphere was stirred at 0° C. for 30 min then 3 hours at room temperature. After work-up (quench with a saturated sodium thiosulfate solution) and purification by chromatography over silica gel, eluting with ethyl acetate in dichloromethane, the desired product was obtained as a colorless oil. LCMS (method 1): 457 (M+H)⁺; retention time: 0.89 min.

### Example E3: Preparation of 2-(5-tert-butoxy-3-ethylsulfonyl-2-pyridyl)-3-methyl-6-(trifluoromethyl)imidazo[4,5-c]pyridine (Compound P5)

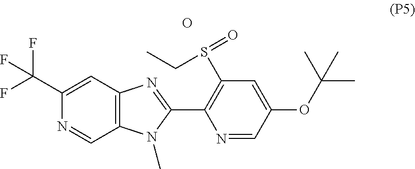

### Step 1: Preparation of 2-(5-tert-butoxy-3-ethylsulfanyl-2-pyridyl)-3-methyl-6-(trifluoromethyl)imidazo[4,5-c]pyridine (Compound P3)

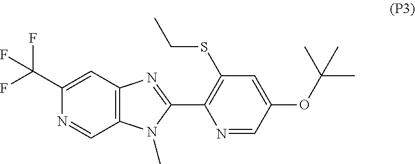 [SMILES: CCSc1cc(OC(C)(C)C)cnc1-c1nc2cc(C(F)(F)F)ncc2n1C]

A microwave vial was charged with 2-(5-bromo-3-ethylsulfanyl-2-pyridyl)-3-methyl-6-(trifluoromethyl)imidazo[4,5-c]pyridine (prepared according to WO16096584) (250 mg, 0.60 mmol), toluene (1.5 ml) and [P(tBu)₃]Pd(crotyl)Cl (12 mg, 0.03 mmol, 0.05 equiv). After degassing with argon for 5 min, sodium tert-butoxide (64 mg, 0.60 mmol, 1.10 equiv.) was added. The vessel was sealed and heated in an oil bath at 130° C. overnight. After cooling to room temperature, the reaction mixture was poured into water, and the aqueous phase was extracted twice with ethyl acetate. The combined organic phases were washed with brine, dried over sodium sulfate, filtered and concentrated under vacuum. Purification over silica gel eluting with ethyl acetate in dichloromethane afforded the title compound as a yellow solid. LCMS (method 1): 411 (M+H)⁺; retention time: 1.10 min.

### Step 2: Preparation of 2-(5-tert-butoxy-3-ethylsulfonyl-2-pyridyl)-3-methyl-6-(trifluoromethyl)imidazo[4,5-c]pyridine (Compound P5)

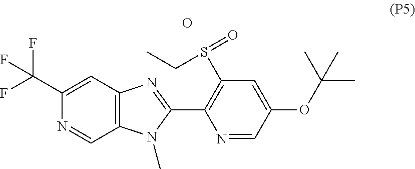

According to the same procedure described above in step 2 for example E1, a mixture of 2-(5-tert-butoxy-3-ethylsulfanyl-2-pyridyl)-3-methyl-6-(trifluoromethyl)imidazo[4,5-c]pyridine (250 mg, 0.61 mmol) and 3-chloroperbenzoic acid (231 mg, 1.34 mmol, 2.2 equiv.) in dichloromethane (4.9 ml) under argon atmosphere was stirred at 0° C. for 30 min then 3 hours at room temperature. After work-up (quench with a saturated sodium thiosulfate solution) and two purifications by chromatography over silica gel, eluting with ethyl acetate in dichloromethane, the desired product was obtained as a white foam. LCMS (method 1): 443 (M+H)⁺; retention time: 1.04 min.

### Example E4: Preparation of 2-[[5-ethylsulfonyl-6-[3-methyl-6-(trifluoromethyl)imidazo[4,5-c]pyridin-2-yl]-3-pyridyl]oxy]-2,2-difluoro-N,N-dimethyl-acetamide (Compound P6)

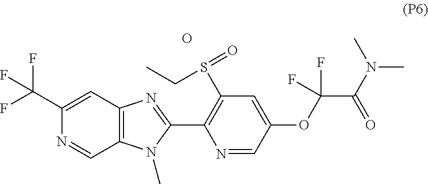

### Step 1: Preparation of 5-ethylsulfonyl-6-[3-methyl-6-(trifluoromethyl)imidazo[4,5-c]pyridin-2-yl]pyridin-3-ol (Compound II)

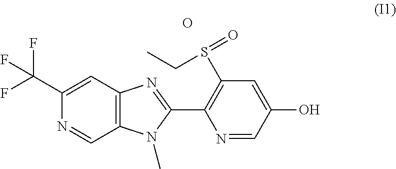

A 35% mass hydrogen peroxide solution (17 μI, 0.20 mmol, 1.0 equiv.) was added to a mixture of 2-[3-ethylsulfonyl-5-(4,4,5,5-tetramethyl-1,3,2-dioxaborolan-2-yl)-2-pyridyl]-3-methyl-6-(trifluoromethyl)imidazo[4,5-c]pyridine (prepared as described in WO 2016/005263) (100 mg, 0.20 mmol) in tetrahydrofuran (2.0 ml). After stirring at room temperature overnight, the reaction mixture was quenched with a saturated sodium thiosulfate solution and a 1M sodium hydroxide solution. The aqueous phase was extracted with ethyl acetate, the combined organic phases were washed with brine, dried over sodium sulfate, filtered and concentrated. Purification of the crude material over silica gel eluting with methanol in dichloromethane, followed by concentration of the selected fractions, afforded the title compound as a yellow solid. LCMS (method 1): 387 (M+H)⁺; retention time: 0.77 min.

### Step 2: Preparation of 2-[[5-ethylsulfonyl-6-[3-methyl-6-(trifluoromethyl)imidazo[4,5-c]pyridin-2-yl]-3-pyridyl]oxy]-2,2-difluoro-N,N-dimethyl-acetamide (compound P6)

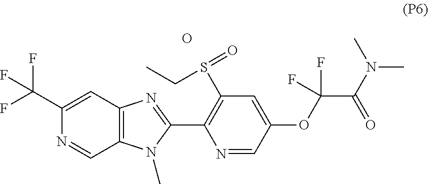

To a stirred mixture of 5-ethylsulfonyl-6-[3-methyl-6-(trifluoromethyl)imidazo[4,5-c]pyridin-2-yl]pyridin-3-ol (compound I1) (309 mg, 0.800 mmol, 1.00 equiv.) and potassium carbonate (420 mg, 3.00 mmol, 3.75 equiv.) in N,N-dimethylacetamide (3.0 ml) was added 2-bromo-2,2-difluoro-N,N-dimethylacetamide (500 mg, 2.50 mmol, 3.12 equiv.) at room temperature. The obtained mixture was heated to 120° C. for 10 h. After cooling to room temperature, the reaction mixture was diluted with water and extracted with ethyl acetate (3×). The combined organic layers were washed with brine, dried (Na₂SO₄) and concentrated under reduced pressure. Purification of the crude material over silica gel eluting with ethyl acetate/hexanes followed by concentration of the selected fractions afforded the title compound as a white solid. Mp. 178-180° C.

### Example E5: Preparation of 2-[3-ethylsulfonyl-5-(2-methylsulfonylethoxy)-2-pyridyl]-3-methyl-6-(trifluoromethyl)imidazo[4,5-c]pyridine (Compound P7)

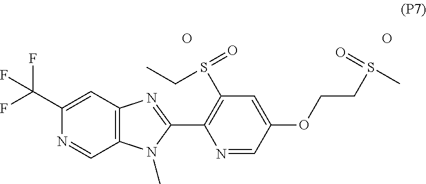

### Step 1: Preparation of 2-[3-ethylsulfonyl-5-(2-methylsulfanylethoxy)-2-pyridyl]-3-methyl-6-(trifluoromethyl)imidazo[4,5-c]pyridine (Compound P11)

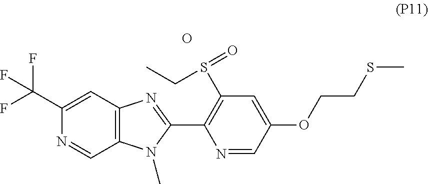

Cesium carbonate (400 mg, 3.0 mmol, 4.0 equiv.) and 1-bromo-2-methylsulfanyl-ethane (200 mg, 1.0 mmol, 1.5 equiv.) were successively added to a mixture of 5-ethylsulfonyl-6-[3-methyl-6-(trifluoromethyl)imidazo[4,5-c]pyridin-2-yl]pyridin-3-ol (compound 11, prepared as described above in step 1 for example P6) (300 mg, 0.8 mmol) in N,N-dimethylformamide (4.0 ml). After stirring at room temperature overnight, the reaction mixture was poured into water, and the aqueous phase was extracted with ethyl acetate. The combined organic phases were washed with brine, dried over sodium sulfate, and concentrated under reduced pressure. The crude material was purified by chromatography over silica gel eluting with ethyl acetate in hexanes, and concentration of the selected fractions afforded the title compound. LCMS (method 4): 461 (M+H)⁺; retention time: 1.05 min.

### Step 2: Preparation of 2-[3-ethylsulfonyl-5-(2-methylsulfanylethoxy)-2-pyridyl]-3-methyl-6-(trifluoromethyl)imidazo[4,5-c]pyridine (Compound P7)

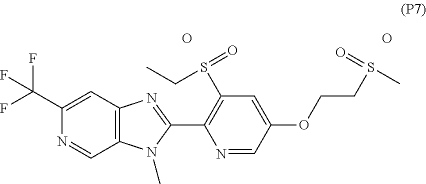

According to the same procedure as in step 2 for example E1, 2-[3-ethylsulfonyl-5-(2-methylsulfanylethoxy)-2-pyridyl]-3-methyl-6-(trifluoromethyl)imidazo[4,5-c]pyridine (200 mg, 0.40 mmol) was treated with 3-chloroperbenzoic acid (200 mg, 0.87 mmol, 2.2 equiv.) in dichloromethane (4.0 ml) under argon atmosphere at 0° C. for 15 min then 2 hours at room temperature. After work-up and purification by chromatography over silica gel, eluting with ethyl acetate in hexanes, the desired product was obtained. LCMS (method 3): 493 (M+H)⁺; retention time: 1.26 min.

### Example E6: Preparation of_2-[3-ethylsulfonyl-5-(2-methoxyethoxy)-2-pyridyl]-3-methyl-6-(trifluoromethyl)imidazo[4,5-c]pyridine (Compound P8)

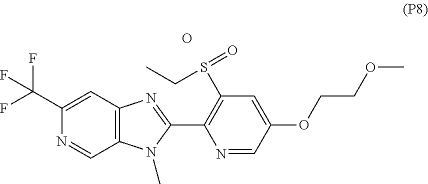

According to the same procedure described in step 1 for example E5, 5-ethylsulfonyl-6-[3-methyl-6-(trifluoromethyl)imidazo[4,5-c]pyridin-2-yl]pyridin-3-ol (compound 11, prepared as described above in step 1 for example P6) (100 mg, 0.26 mmol) was treated with 1-bromo-2-methoxy-ethane (50 mg, 0.36 mmol, 1.4 equiv.) using potassium carbonate (100 mg, 0.72 mmol, 4.0 equiv.) in N,N-dimethylformamide (2.0 ml). After work-up and purification by chromatography over silica gel eluting with ethyl acetate in hexanes, concentration of the selected fractions afforded the title compound. LCMS (method 4): 445 (M+H)⁺; retention time: 0.94 min.

### Example E7: Preparation of 2-[[5-ethylsulfonyl-6-[3-methyl-6-(trifluoromethyl)imidazo[4,5-c]pyridin-2-yl]-3-pyridyl]oxy]-2-methyl-propanenitrile (Compound P18)

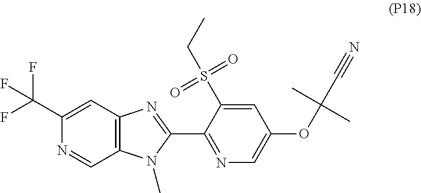 [SMILES: CCS(=O)(=O)c1cc(OC(C)(C)C#N)cnc1-c1nc2cc(C(F)(F)F)ncc2n1C]

### Step 1: Preparation of 2-[[5-ethylsulfanyl-6-[3-methyl-6-(trifluoromethyl)imidazo[4,5-c]pyridin-2-yl]-3-pyridyl]oxy]acetonitrile (Compound P21)

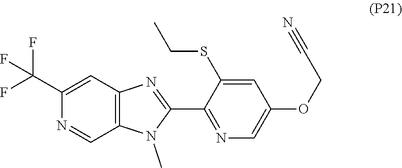 [SMILES: CCSc1cc(OCC#N)cnc1-c1nc2cc(C(F)(F)F)ncc2n1C]

To a vial charged with 5-ethylsulfanyl-6-[3-methyl-6-(trifluoromethyl)imidazo[4,5-c]pyridin-2-yl]pyridin-3-ol (prepared according to WO16096584) (0.5 g, 1.411 mmol) and potassium carbonate (0.2967 g, 2.116 mmol) in N,N-dimethylformamide (10 mL) was added bromoacetonitrile (0.152 mL, 2.11 mmol). The resulting mixture was stirred at room temperature for 1.5 hours. The reaction mixture was quenched with aqueous NaHCO₃. The aqueous layer was extracted twice with ethylacetate, and the combined organic layer were washed with aqueous NaHCO₃, dry with Na₂SO₄, filtered and evaporated under reduced pressure. The crude material was purified by chromatography over silica gel to give 2-[[5-ethylsulfanyl-6-[3-methyl-6-(trifluoromethyl)imidazo[4,5-c]pyridin-2-yl]-3-pyridyl]oxy]acetonitrile.

LCMS (method 1): 394 (M+H)⁺; retention time: 0.94 min.

¹⁹F NMR (377 MHz, CHLOROFORM-d) δ ppm −66.23 −−66.09 (m, 3 F)

¹H NMR (400 MHz, CHLOROFORM-d) δ ppm 1.38 (t, J=7.34 Hz, 3H) 2.95-3.02 (m, 2H) 4.01-4.12 (m, 3H) 4.90-4.99 (m, 2H) 7.37 (d, J=2.57 Hz, 1H) 8.16-8.23 (m, 1H) 8.24-8.33 (m, 1H) 8.91-9.01 (m, 1H)

### Step 2: Preparation of 2-[[5-ethylsulfanyl-6-[3-methyl-6-(trifluoromethyl)imidazo[4,5-c]pyridin-2-yl]-3-pyridyl]oxy]-2-methyl-propanenitrile (Compound P19)

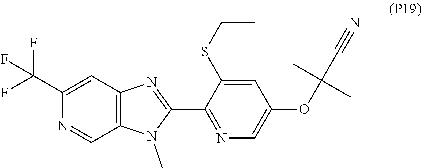 [SMILES: CCSc1cc(OC(C)(C)C#N)cnc1-c1nc2cc(C(F)(F)F)ncc2n1C]

2-[[5-ethylsulfanyl-6-[3-methyl-6-(trifluoromethyl)imidazo[4,5-c]pyridin-2-yl]-3-pyridyl]oxy]acetonitrile (0.29 g, 0.74 mmol) and iodomethane (0.184 mL, 2.95 mmol) were dissolved in tetrahydrofuran (4.5 mL). The solution obtained was cooled to 0-5° C. and sodium bis(trimethylsilyl)amide (1.0 mol/L in THF, 2.2 ml, 2.2 mmol) was added dropwise during 5 min. The resulting mixture was stirred 2 hours at 5° C. The reaction mixture was quenched with aqueous NaHCO₃. The aqueous layer was extracted twice with ethylacetate, and the combined organic layer were washed with aqueous NaHCO₃, dried (Na₂SO₄), filtered and evaporated under reduced pressure. The crude material was purified by chromatography over silica gel to give 2-[[5-ethylsulfanyl-6-[3-methyl-6-(trifluoromethyl)imidazo[4,5-c]pyridin-2-yl]-3-pyridyl]oxy]-2-methyl-propanenitrile. LCMS (method 1): 422 (M+H)⁺; retention time: 1.02 min.

¹⁹F NMR (377 MHz, CHLOROFORM-d) δ ppm −66.19 −−66.10 (m, 3F)

¹H NMR (400 MHz, CHLOROFORM-d) δ ppm 1.33-1.43 (m, 3H) 1.85 (s, 6H) 2.94-3.05 (m, 2H) 4.04-4.11 (m, 3H) 7.63-7.70 (m, 1H) 8.17-8.24 (m, 1H) 8.29-8.39 (m, 1H) 8.93-9.01 (m, 1H)

### Step 3: Preparation of 2-[[5-ethylsulfonyl-6-[3-methyl-6-(trifluoromethyl)imidazo[4,5-c]pyridin-2-yl]-3-pyridyl]oxy]-2-methyl-propanenitrile (Compound P18)

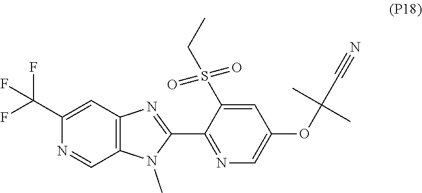 [SMILES: CCS(=O)(=O)c1cc(OC(C)(C)C#N)cnc1-c1nc2cc(C(F)(F)F)ncc2n1C]

According to the same procedure as in step 2 for example E1, 2-[[5-ethylsulfanyl-6-[3-methyl-6-(trifluoromethyl)imidazo[4,5-c]pyridin-2-yl]-3-pyridyl]oxy]-2-methyl-propanenitrile (106 mg, 0.251 mmol) was treated with 3-chloroperbenzoic acid (127 mg, 0.553 mmol, 2.20 equiv.) in dichloromethane (1.5 ml) under argon atmosphere at 0° C. for 15 min then 2 hours at room temperature. After work-up and purification by chromatography over silica gel, eluting with ethyl acetate in hexanes, the desired product was obtained. LCMS (method 1): 454 (M+H)⁺; retention time: 0.97 min.

¹⁹F NMR (377 MHz, CHLOROFORM-d) δ ppm −66.23 −−66.16 (m, 3 F).

¹H NMR (400 MHz, CHLOROFORM-d) δ ppm 1.34-1.44 (m, 3H) 1.86-1.95 (m, 6H) 3.81 (q, J=7.58 Hz, 2H) 3.88-3.95 (m, 3H) 8.07-8.14 (m, 1H) 8.26-8.34 (m, 1H) 8.81-8.88 (m, 1H) 8.95-9.03 (m, 1H)

### Example E8: Preparation of 2-[3-ethylsulfonyl-5-(3-methylsulfanylpropoxy)-2-pyridyl]-3-methyl-6-(trifluoromethyl)imidazo[4,5-c]pyridine

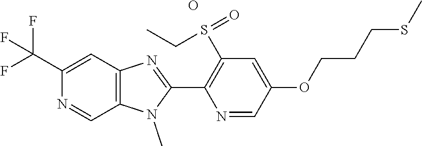

In a microwave vial, 2-(5-bromo-3-ethylsulfonyl-2-pyridyl)-3-methyl-6-(trifluoromethyl)imidazo[4,5-c]pyridine (1 g, 2.226 mmol) was dissolved in methylsulfinylmethane (10 mL), and dipotassium carbonate (1.53 g, 11.13 mmol) was added follow by the addition of 3-methylsulfanylpropan-1-ol (1.17 mL, 11.13 mmol). The reaction vial was placed in microwave and heated at 148 C.° for 1 hour. Completion of reaction was monitored by TLC and LCMS. After completion of reaction, the resulting mixture was poured into water (30 ml), and resultant solution was extracted with ethyl acetate (20 ml*3). The combined organic layer was washed with aqueous saturated sodium chloride solution (30 ml), dried over anhydrous sodium sulfate, and concentrated under reduced pressure. The residue was purified by chromatography over silica gel to afford 2-[3-ethylsulfonyl-5-(3-methylsulfanylpropoxy)-2-pyridyl]-3-methyl-6-(trifluoromethyl)imidazo[4,5-c]pyridine.

¹H NMR (400 MHz, CHLOROFORM-d) δ ppm 2.15-2.24 (m, 5H) 2.68-2.79 (m, 2H) 3.75-3.88 (m, 5H) 4.34 (t, 2H) 7.96 (d, 1H) 8.08-8.12 (m, 1H) 8.65 (d, 1H) 8.96 (s, 1H)

### Example E9: 2-[5-(1,1-dimethyl-2-methylsulfanyl-ethoxy)-3-ethylsulfonyl-2-pyridyl]-3-methyl-6-(trifluoromethyl)imidazo[4,5-c]pyridine (Compound P50)

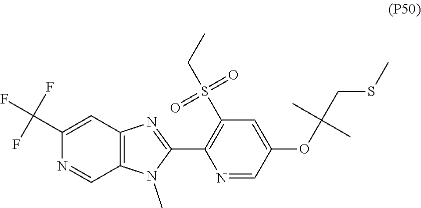 [SMILES: CCS(=O)(=O)c1cc(OC(C)(C)CSC)cnc1-c1nc2cc(C(F)(F)F)ncc2n1C]

### Step 1: Preparation of ethyl 2-[[5-ethylsulfonyl-6-[3-methyl-6-(trifluoromethyl)imidazo[4,5-c]pyridin-2-yl]-3-pyridyl]oxy]-2-methyl-propanoate (Compound P57)

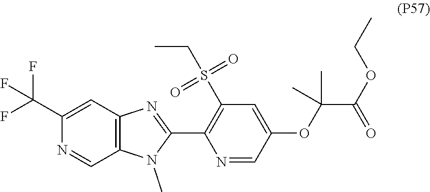 [SMILES: CCOC(=O)C(C)(C)Oc1cnc(-c2nc3cc(C(F)(F)F)ncc3n2C)c(S(=O)(=O)CC)c1]

To a solution of 5-ethylsulfonyl-6-[3-methyl-6-(trifluoromethyl)imidazo[4,5-c]pyridin-2-yl]pyridin-3-ol (0.1 g, 0.2588 mmol) in acetonitrile (2.0 mL) was added dicesium; carbonic acid (0.1273 g, 0.3882 mmol) and ethyl 2-bromo-2-methyl-propanoate (0.04938 mL, 0.3364 mmol). The reaction mixture was stirred at room temperature overnight under nitrogen atmosphere, monitored by TLC. The reaction mass was diluted with water (10 ml), extracted with ethyl acetate (3*20 ml). The combined organic layer was dried over sodium sulfate, filtered and concentrated under reduced pressure. Crude product was purified by chromatography over silica gel to afford ethyl 2-[[5-ethylsulfonyl-6-[3-methyl-6-(trifluoromethyl)imidazo[4,5-c]pyridin-2-yl]-3-pyridyl]oxy]-2-methyl-propanoate.

LCMS: retention time 1.48 min, 501.14 (M+H)

1H NMR (400 MHz, CHLOROFORM-d) b ppm 1.31-1.37 (m, 6H) 1.73-1.79 (m, 6H) 3.73-3.81 (m, 2H) 3.87 (s, 3H) 4.31 (q, 2H) 7.86 (d, 1H) 8.08-8.11 (m, 1H) 8.55 (d, 1H) 8.96 (s, 1H)

### Step 2: Preparation of 2-[[5-ethylsulfonyl-6-[3-methyl-6-(trifluoromethyl)imidazo[4,5-c]pyridin-2-yl]-3-pyridyl]oxy]-2-methyl-propan-1-ol (Compound 14)

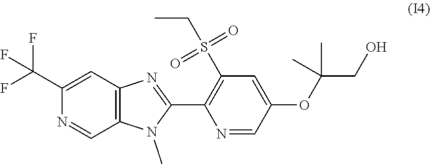 [SMILES: CCS(=O)(=O)c1cc(OC(C)(C)CO)cnc1-c1nc2cc(C(F)(F)F)ncc2n1C]

To a 0° C. cooled solution of ethyl 2-[[5-ethylsulfonyl-6-[3-methyl-6-(trifluoromethyl)imidazo[4,5-c]pyridin-2-yl]-3-pyridyl]oxy]-2-methyl-propanoate (0.5 g, 0.9990 mmol) in methanol (10 mL) was added sodium borohydride (0.1968 g, 4.995 mmol) in several portions. The reaction mixture was stirred at room temperature for 4 hours and monitored by TLC. Sodium borohydride (0.1968 g, 4.995 mmol) was added. again at 0° C. and reaction mixture was stirred at room temperature overnight to be completed. Reaction mass was quenched with 2N HCl (10 ml) and extracted with ethyl acetate (3*20 ml). Combined organic layer was dried over sodium sulfate, filtered and concentrated under reduced pressure. Crude product was purified by chromatography to afford 2-[[5-ethylsulfonyl-6-[3-methyl-6-(trifluoromethyl)imidazo[4,5-c]pyridin-2-yl]-3-pyridyl]oxy]-2-methyl-propan-1-ol.

LCMS: retention time 0.81 min, 459 (M+H)

1H NMR (400 MHz, CHLOROFORM-d) δ ppm 1.35-1.40 (m, 3H) 1.41-1.45 (m, 6H) 3.74-3.84 (m, 2H) 3.84-3.89 (m, 3H) 4.05 (s, 2H) 7.99 (d, J=2.81 Hz, 1H) 8.09-8.12 (m, 1H) 8.70 (d, J=2.81 Hz, 1H) 8.97 (s, 1H)

### Step 3: Preparation of [2-[[5-ethylsulfonyl-6-[3-methyl-6-(trifluoromethyl)imidazo[4,5-c]pyridin-2-yl]-3-pyridyl]oxy]-2-methyl-propyl] trifluoromethanesulfonate (Compound 15)

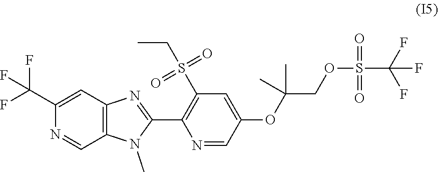 [SMILES: CCS(=O)(=O)c1cc(OC(C)(C)COS(=O)(=O)C(F)(F)F)cnc1-c1nc2cc(C(F)(F)F)ncc2n1C]

To a 0° C. cooled solution of 2-[[5-ethylsulfonyl-6-[3-methyl-6-(trifluoromethyl)imidazo[4,5-c]pyridin-2-yl]-3-pyridyl]oxy]-2-methyl-propan-1-ol (0.15 g, 0.3272 mmol) in dichloromethane (4.0 mL) was added pyridine (0.165 mL, 1.963 mmol). trifluoromethylsulfonyl trifluoromethanesulfonate (0.1101 mL. 0.6545 mmol) was then added drop wise to this solution always at 0° C. The reaction mixture was stirred at 0° C. for 30 min and then diluted with water (10 ml). The aqueous layer was extracted with dichloromethane (3*20 ml). The combined organic layer was dried over sodium sulfate, filtered and concentrated under reduced pressure to afford crude. The crude was used without further purification for the next step.

LCMS: retention time 1.07 min, 591.69 (M+H)

### Step 4: Preparation of 2-[5-(1,1-dimethyl-2-methylsulfanyl-ethoxy)-3-ethylsulfonyl-2-pyridyl]-3-methyl-6-(trifluoromethyl)imidazo[4,5-c]pyridine (Compound P50)

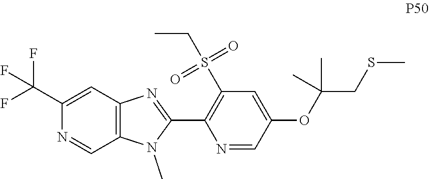 [SMILES: CCS(=O)(=O)c1cc(OC(C)(C)CSC)cnc1-c1nc2cc(C(F)(F)F)ncc2n1C]

To a 0° C. cooled solution of [2-[[5-ethylsulfonyl-6-[3-methyl-6-(trifluoromethyl)imidazo[4,5-c]pyridin-2-yl]-3-pyridyl]oxy]-2-methyl-propyl] trifluoromethanesulfonate (0.16 g, 0.2710 mmol) in N,N-dimethylformamide (4.0 mL) was added sodium; methanethiolate (0.02089 g, 0.2981 mmol). The reaction mixture was stirred at room temperature for 1 hour, monitored by TLC. After completion, the reaction mass was diluted with water (15 ml), extracted with ethyl acetate (3*20 ml). The combined organic layer was dried over sodium sulfate, filtered and concentrated under reduced pressure. Crude product was purified by chromatography over silica gel to afford 2-[5-(1,1-dimethyl-2-methylsulfanyl-ethoxy)-3-ethylsulfonyl-2-pyridyl]-3-methyl-6-(trifluoromethyl)imidazo[4,5-c]pyridine

LCMS: retention time 1.08 min, 489.13 (M+H)

1H NMR (400 MHz, CHLOROFORM-d) d=9.00 (s, 1H), 8.69 (d, 1H), 8.17-8.09 (m, 2H), 3.90 (s, 3H), 3.86-3.71 (m, 3H), 2.92 (s, 2H), 2.25 (s, 3H), 1.58 (s, 6H), 1.42-1.32 (m, 3H)

### Example E10: Preparation of 2-[5-(3,3-difluorocyclobutoxy)-3-ethylsulfonyl-2-pyridyl]-3-methyl-6-(trifluoromethyl)imidazo[4,5-c]pyridine (Compound P42)

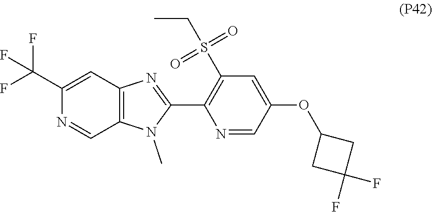 [SMILES: CCS(=O)(=O)c1cc(OC2CC(F)(F)C2)cnc1-c1nc2cc(C(F)(F)F)ncc2n1C]

### Step 1: Preparation of 2-[5-(3-benzyloxycyclobutoxy)-3-ethylsulfonyl-2-pyridyl]-3-methyl-6-(trifluoromethyl)imidazo[4,5-c]pyridine (Compound 16)

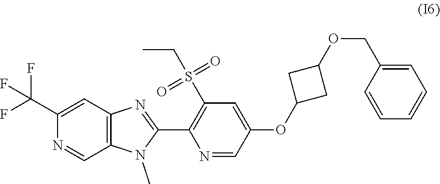 [SMILES: CCS(=O)(=O)c1cc(OC2CC(OCc3ccccc3)C2)cnc1-c1nc2cc(C(F)(F)F)ncc2n1C]

Method 1

In a. solution of 2-(5-bromo-3-ethylsulfonyl-2-pyridyl)-3-methyl-6-(trifluoromethyl)imidazo[4,5-c]pyridine (1 g, 2.226 mmol) in methylsulfinylmethane (6 mL) were added dipotassium; carbonate (8.905 mmol, 1.231 g) and 3-benzyloxycyclobutanol (6.679 mmol, 1.190 g). The reaction vial was placed in microwave and heated at 148° C. for 2 hours. After completion of reaction, reaction mixture was poured into water (60 ml), and the aqueous layer was extracted with ethyl acetate. The combined organic layer was washed with aqueous saturated sodium chloride solution (30 ml), dried over anhydrous sodium sulfate, and concentrated of under reduced pressure. The residue was purify over reduced pressure.

LCMS:—rt 1.13 min, mass 547 (M+1)

1H NMR (400 MHz, CHLOROFORM-d) δ ppm 1.34-1.38 (m, 3H) 2.54-2.81 (m, 3H) 2.85-3.03 (m, 2H) 3.03-3.14 (m, 2H) 3.77-3.83 (m, 2H) 3.87 (s, 3H) 4.07-4.16 (m, 1H) 4.88 (quin, J=6.97 Hz, 1H) 7.79-7.84 (m, 1H) 8.09-8.12 (m, 1H) 8.57 (d, J=2.81 Hz, 1H) 8.95-9.00 (m, 1H).

Method 2

To a solution of 3-benzyloxycyclobutanol (5 g, 28.054 mmol) in dichloromethane (50 mL) was added N,N-diethylethanamine (8.5 mL, 60 mmol). The resulting solution was cooled at 0° C. and methanesulfonyl chloride (4.356 mL, 56.107 mmol) was added slowly. The reaction was stirred at room temperature for 1 hour and then quenched with water. The aqueous layer was extracted with dichloromethane. The combined organic layer was dried over sodium sulfate, filtered and concentrated under reduced pressure to afford (3-benzyloxycyclobutyl) methanesulfonate.

In a solution of 5-ethylsulfonyl-6-[3-methyl-6-(trifluoromethyl)imidazo[4,5-c]pyridin-2-yl]pyridin-3-ol (9.3 g, 24 mmol) in acetonitrile (70 mL) were added dicesium; carbonate (12 g, 36 mmol) and (3-benzyloxycyclobutyl) methanesulfonate (6.2 g, 24 mmol), prepared as described before. The reaction was stirred at room temperature overnight. After completion, reaction mass was quenched with water and extracted with ethylacetate. The combined organic layer was dried over sodium sulfate, filtered and concentrated under reduced pressure to afford 2-[5-(3-benzyloxycyclobutoxy)-3-ethylsulfonyl-2-pyridyl]-3-methyl-6-(trifluoromethyl)imidazo[4,5-c]pyridine.

LCMS:—rt 1.13 min, mass 547 (M+1)

### Step 2: Preparation of 3-[[5-ethylsulfonyl-6-[3-methyl-6-(trifluoromethyl)imidazo[4,5-c]pyridin-2-yl]-3-pyridyl]oxy]cyclobutanol (Compound 17)

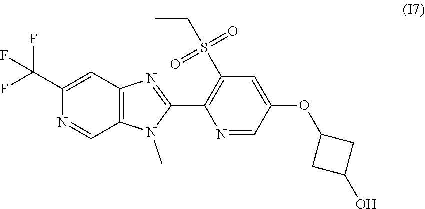 [SMILES: CCS(=O)(=O)c1cc(OC2CC(O)C2)cnc1-c1nc2cc(C(F)(F)F)ncc2n1C]

To a solution of 2-[5-(3-benzyloxycyclobutoxy)-3-ethylsulfonyl-2-pyridyl]-3-methyl-6-(trifluoromethyl)imidazo[4,5-c]pyridine (40 mg, 0.07318 mmol) in methanol (5 mL) was added palladium (20 mg, 0.018793 mmol, 10% On activated carbon, 50% wet) in portions under argon atmosphere, then the reaction mixture was stirred under hydrogen atmosphere, at room temperature for 24 hours. Reaction mixture was filtered over hyflo and washed with methanol (20 mL X 2). The filtrate was concentrated under reduced pressure to afford crude compound, which was purified on silica column to afford 3-[[5-ethylsulfonyl-6-[3-methyl-6-(trifluoromethyl)imidazo[4,5-c]pyridin-2-yl]-3-pyridyl]oxy]cyclobutanol.

LCMS:—rt 0.86 min 457.12 (M+1)

1H NMR (400 MHz, CHLOROFORM-d) δ ppm 1.35 (t, J=7.46 Hz, 3H) 2.50-2.66 (m, 4H) 3.70-3.82 (m, 2H) 3.87 (s, 3H) 4.67-4.74 (m, 1H) 5.07-5.14 (m, 1H) 7.81-7.83 (m, 1H) 8.10 (s, 1H) 8.54-8.56 (m, 1H) 8.97 (s, 1H)

### Step 3: Preparation of 3-[[5-ethylsulfonyl-6-[3-methyl-6-(trifluoromethyl)imidazo[4,5-c]pyridin-2-yl]-3-pyridyl]oxy]cyclobutanone (Compound 18)

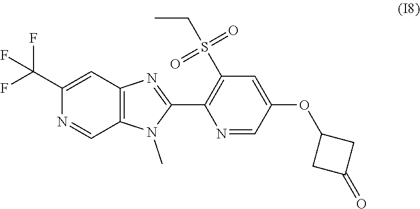 [SMILES: CCS(=O)(=O)c1cc(OC2CC(=O)C2)cnc1-c1nc2cc(C(F)(F)F)ncc2n1C]

To a solution of 3-[[5-ethylsulfonyl-6-[3-methyl-6-(trifluoromethyl)imidazo[4,5-c]pyridin-2-yl]-3-pyridyl]oxy]cyclobutanol (100 mg, 0.2191 mmol) in dichloromethane (5 mL) was added (1,1-diacetoxy-3-oxo-1$l\hat{ }{5},2-benziodoxol-1-yl) acetate (139 mg, 0.3287 mmol) and the reaction mixture was stirred for 3 hours until completion, monitored by TLC and LCMS. The reaction mass was poured into water (10 ml), and quenched with bicarbonate. The aqueous layer r was extracted with ethyl acetate (20 ml*3). The combined organic layer was washed with aqueous saturated sodium chloride solution (10 ml), dried over anhydrous sodium sulfate, and concentrated under reduced pressure. The crude was chromatographed over silica gel to afford 3-[[5-ethylsulfonyl-6-[3-methyl-6-(trifluoromethyl)imidazo[4,5-c]pyridin-2-yl]-3-pyridyl]oxy]cyclobutanone.

LCMS: rt 0.91 min 455.11 mass (M+1)

1H NMR (400 MHz, CHLOROFORM-d) δ ppm 1.33-1.41 (m, 4H) 3.37-3.47 (m, 2H) 3.65-3.74 (m, 2H) 3.74-3.87 (m, 2H) 3.87-3.92 (m, 3H) 5.20-5.27 (m, 1H) 7.93 (d, J=2.81 Hz, 1H) 8.11 (s, 1H) 8.65 (d, J=2.81 Hz, 1H) 8.98 (s, 1H)

### Step 4: Preparation of 2-[5-(3,3-difluorocyclobutoxy)-3-ethylsulfonyl-2-pyridyl]-3-methyl-6-(trifluoromethyl)imidazo[4,5-c]pyridine (Compound P42)

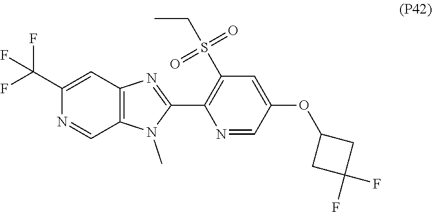 [SMILES: CCS(=O)(=O)c1cc(OC2CC(F)(F)C2)cnc1-c1nc2cc(C(F)(F)F)ncc2n1C]

A solution of 3-[[5-ethylsulfonyl-6-[3-methyl-6-(trifluoromethyl)imidazo[4,5-c]pyridin-2-yl]-3-pyridyl]oxy]cyclobutanone (500 mg, 1.100 mmol) in dichloromethane (15 mL) was cooled down at −78° C. and N-ethyl-N-(trifluoro-$l\hat{ }{4}-sulfanyl)ethanamine (4.401 mmol, 0.5815 mL) was added drop wise to this cooled solution. The resulting mixture was stirred for 30 min then warm up at room temperature and stirred 3 hours. Completion of reaction was monitored by TLC and LCMS. After completion, reaction mixture was quenched in solid Na₂CO₃, water was added and the resulting solution was extracted with dichloromethane (80 ml*3). The combined organic layer were dried over anhydrous sodium sulfate, and concentrated under reduced pressure. The crude was purify by chromatography over silica gel to afford 2-[5-(3,3-difluorocyclobutoxy)-3-ethylsulfonyl-2-pyridyl]-3-methyl-6-(trifluoromethyl)imidazo[4,5-c]pyridine.

LCMS:—rt 1.05 min, 477 (M+1)

¹H NMR (400 MHz, CHLOROFORM-d) δ ppm 1.37 (t, J=7.46 Hz, 3H) 2.83-2.97 (m, 2H) 3.20-3.32 (m, 2H) 3.75-3.87 (m, 2H) 3.88 (s, 3H) 4.90 (br d, J=7.09 Hz, 1H) 7.86 (d, J=2.69 Hz, 1H) 8.10 (s, 1H) 8.60 (d, J=2.81 Hz, 1H) 8.98 (s, 1H)

### Example E11: Preparation of 2-[3-ethylsulfonyl-5-[2-fluoro-1-(fluoromethyl)ethoxy]-2-pyridyl]-3-methyl-6-(trifluoromethyl)imidazo[4,5-c]pyridine (Compound P53)

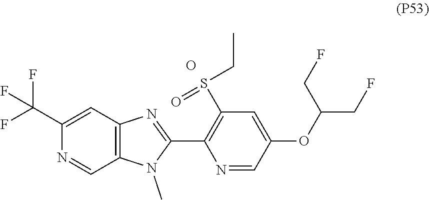

In a solution of 2-(5-bromo-3-ethylsulfonyl-2-pyridyl)-3-methyl-6-(trifluoromethyl)imidazo[4,5-c]pyridine (0.6 g, 1 mmol) in methylsulfinylmethane (8 mL), dipotassium; carbonate (0.7 g, 5 mmol) and 1,3-difluoropropan-2-ol (0.5 g, 5 mmol) were added The reaction was stirred in microwave at 148° C. for 1 hour. Then the reaction was poured into water (30 ml), and. the resultant solution was extracted with ethyl acetate (20 ml*3). The combined organic layer was washed with aqueous saturated sodium chloride solution (30 ml), dried over anhydrous sodium sulfate, and concentrated under reduced pressure. The crude was purify by chromatography over silica gel to afford 2-[3-ethylsulfonyl-5-[2-fluoro-1-(fluoromethyl)ethoxy]-2-pyridyl]-3-methyl-6-(trifluoromethyl)imidazo[4,5-c]pyridine.

LCMS: 465 M+rt 0.95 MIN

1H NMR (400 MHz, CHLOROFORM-d) δ ppm 1.37 (t, 3H) 3.77-3.90 (m, 5H) 4.69-4.89 (m, 4H) 4.97-5.11 (m, 1H) 8.06-8.12 (m, 2H) 8.74 (d, 1H) 8.98 (s, 1H)

### Example E12: Preparation of 1-[[5-ethylsulfonyl-6-[3-methyl-6-(trifluoromethyl)imidazo[4,5-c]pyridin-2-yl]-3-pyridyl]oxymethyl]cyclopropanecarbonitrile (Compound P79)

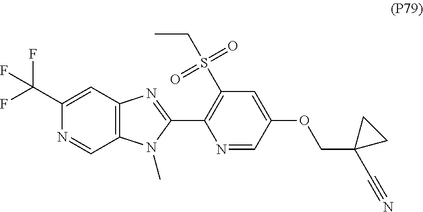 [SMILES: CCS(=O)(=O)c1cc(OCC2(C#N)CC2)cnc1-c1nc2cc(C(F)(F)F)ncc2n1C]

2-(5-bromo-3-ethylsulfonyl-2-pyridyl)-3-methyl-6-(trifluoromethyl)imidazo[4,5-c]pyridine (100 mg, 0.2 mmol) was dissolved in methylsulfinylmethane (3 mL). Dipotassium carbonate (0.2 g, 1 mmol) and 1-(hydroxymethyl)cyclopropanecarbonitrile (0.9 mmol, 0.09 g) were added reaction vial was stirred in microwave a 148° C. for 1 hour. The reaction mixture was poured into water (30 ml) and the aqueous layer was extracted with ethyl acetate. The combined organic layer was washed with aqueous saturated sodium chloride solution (30 ml), dried over anhydrous sodium sulfate, and concentrated under reduced pressure. The residue purify by chromatography over silica gel to afford 1-[[5-ethylsulfonyl-6-[3-methyl-6-(trifluoromethyl)imidazo[4,5-c]pyridin-2-yl]-3-pyridyl]oxymethyl]cyclopropanecarbonitrile.

LCMS: 467 MH+(rt 0.91 min)

1H NMR (400 MHz, CHLOROFORM-d) δ ppm 1.21-1.26 (m, 2H) 1.37 (t, 3H) 1.54-1.59 (m, 1H) 1.58-1.58 (m, 1H) 3.78-3.89 (m, 5H) 4.19 (s, 2H) 7.94 (d, 1H) 8.11 (s, 1H) 8.71 (d, 1H) 8.98 (s, 1H)

### Example E13: Preparation of 2-[3-ethylsulfonyl-4-[3-methyl-6-(trifluoromethyl)imidazo[4,5-c]pyridin-2-yl]phenoxy]-2-methyl-propanenitrile (Compound P103)

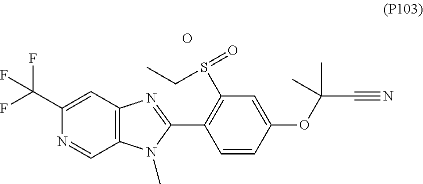

### Step 1: Preparation of 2-[3-ethylsulfonyl-4[3-methyl-6-(trifluoromethyl)imidazo[4,5-c]pyridin-2-yl]phenoxy]-2-methyl-propanamide (Compound 19)

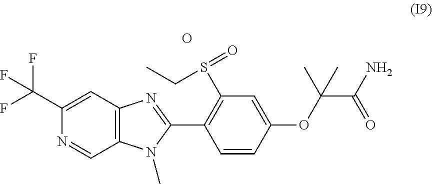

3-ethylsulfonyl-4-[3-methyl-6-(trifluoromethyl)imidazo[4,5-c]pyridin-2-yl]phenol (0.3 g, 0.8 mmol) was dissolved in acetonitrile (4 mL). dicesium; carbonate (0.3 g, 0.9 mmol) and 2-bromo-2-methyl-propanamide (0.1 g, 0.9 mmol) were added at room temperature. The resulting mixture was heated at 70° C. overnight. Reaction was monitored by TLC. dicesium; carbonate (0.15 g, 0.45 mmol) and 2-bromo-2-methyl-propanamide (0.1 g, 0.9 mmol) were added again and reaction mixture was stirred 4 hours at 70° C. until completion. Acetonitrile was concentrated on rotavap, The reaction mixture was acidified with 2N HCl (dropwise addition) and water was added. The aqueous layer was extracted with ethylacetate. The organic layer was washed with brine 10 ml, dried over Na₂SO₄, concentrated to afford 2-[3-ethylsulfonyl-4-[3-methyl-6-(trifluoromethyl)imidazo[4,5-c]pyridin-2-yl]phenoxy]-2-methyl-propanamide.

LCMS—RT1.29 (min), 471 (M+H)

1H NMR (400 MHz, CHLOROFORM-d) δ ppm 1.26 (t, J=7.40 Hz, 5H) 1.69 (s, 6H) 1.95-1.99 (m, 7H) 3.37 (br d, J=7.46 Hz, 2H) 3.76 (s, 3H) 5.41-5.44 (m, 1H) 5.65-5.83 (m, 2H) 5.88 (br s, 1H) 6.47 (br s, 2H) 7.34 (dd, J=8.25, 2.51 Hz, 1H) 7.48 (d, J=8.19 Hz, 1H) 7.79 (d, J=2.45 Hz, 1H) 8.08 (s, 1H) 8.95 (s, 1H)

### Step 2: Preparation of 2-[3-ethylsulfonyl-4-[3-methyl-6-(trifluoromethyl)imidazo[4,5-c]pyridin-2-yl]phenoxy]-2-methyl-propanenitrile

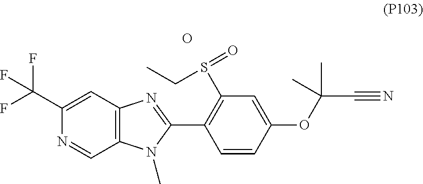

2-[3-ethylsulfonyl-4-[3-methyl-6-(trifluoromethyl)imidazo[4,5-c]pyridin-2-yl]phenoxy]-2-methyl-propanamide (0.3 g, 0.6 mmol) was suspended in dichloromethane (6 mL) and triethylamine (0.4 mL, 3 mmol) was added. The mixture was cooled down at 0° C. and trifluoroacetic anhydride (0.4 mL, 3 mmol) was added dropwise. The resulting suspension was stirred at room temperature. After 2 hours, reaction mixture was carefully quenched with sat.NaHCO₃ to adjust pH basic, and then the aqueous layer was extracted with dichloromethane. The combined organic layer washed with brine, dried over sodium sulfate, filtered and evaporated. Crude was purified by chromatography over silica gel to afford pure product as white solid, 2-[3-ethylsulfonyl-4-[3-methyl-6-(trifluoromethyl)imidazo[4,5-c]pyridin-2-yl]phenoxy]-2-methyl-propanenitrile.

LCMS:—RT 1.44 min, 453.00 (M+H)

1H NMR (400 MHz, CHLOROFORM-d) δ ppm 8.95 (1H, s) 8.09 (1H, s) 7.95 (1H, d) 7.68 (1H, dd) 7.57 (1H, d) 3.77 (3H, s) 3.39 (2H, br d) 1.84-1.91 (6H, m) 1.23-1.32 (4H, m)

### Example E14: Preparation of 2-[2-ethylsulfonyl-4-(methylsulfanylmethoxy)phenyl]-3-methyl-6-(trifluoromethyl)imidazo[4,5-c]pyridine (Compound P106)

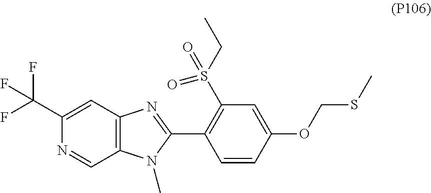 [SMILES: CCS(=O)(=O)c1cc(OCSC)ccc1-c1nc2cc(C(F)(F)F)ncc2n1C]

To a stirred solution of 3-ethylsulfonyl-4-[3-methyl-6-(trifluoromethyl)imidazo[4,5-c]pyridin-2-yl]phenol (0.2 g, 0.5 mmol) in N,N-dimethylformamide (3 mL) was added dipotassium carbonate (0.2 g, 2 mmol) at room temperature and stirred for 5 minutes. Then chloro(methylsulfanyl)methane (0.1 g, 1 mmol) was added dropwise at room temperature. The resulting reaction was stirred at room temperature. 40 ml ice water was added to the reaction mixture and the product was extracted with ethylacetate. The combined organic layers were washed with water (20 ml×3), brine solution, dried over sodium sulphate and concentrated. The crude was then purified by chromatography on silica gel to afford 2-[2-ethylsulfonyl-4-(methylsulfanylmethoxy)phenyl]-3-methyl-6-(trifluoromethyl)imidazo[4,5-c]pyridine (E, 100 mass %) as white solid.

LCMS—RT 1.41 (min), 445.9 (M+H)

1H NMR (400 MHz, CHLOROFORM-d) δ ppm 8.93 (1H, s) 8.09 (1H, s) 7.76 (1H, d) 7.49 (1H, d) 7.36 (1H, dd) 5.32 (2H, s) 3.75 (3H, s) 3.38 (2H, br d) 2.31 (3H, s) 1.27 (4H, t)

### Example H15: Preparation of 2-[2-ethylsulfonyl-4-(methylsulfonylmethoxy)phenyl]-3-methyl-6-(trifluoromethyl)imidazo[4,5-c]pyridine (Compound P105)

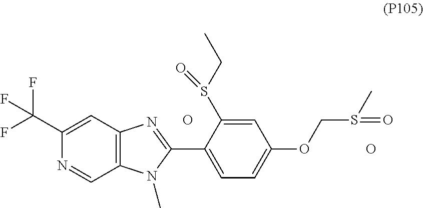

To a solution of 2-[2-ethylsulfonyl-4-(methylsulfanylmethoxy)phenyl]-3-methyl-6-(trifluoromethyl)imidazo[4,5-c]pyridine (0.2 g, 0.4 mmol) in dichloromethane (3 mL) was added portionwise 3-chlorobenzenecarboperoxoic acid (0.2 g, 0.9 mmol) at room temperature. The mixture was stirred at room temperature overnight. 1M solution of sodium hydroxide was added dropwise to adjust the pH basic, and water was added. The aqueous layer was extracted with dichloromethane. Organic phase was separated and washed with brine, dried over Na₂SO₄ and concentrated under vacuum to afford crude product which was then purified by column chromatography on silica gel to afford white solid 2-[2-ethylsulfonyl-4-(methylsulfonylmethoxy)phenyl]-3-methyl-6-(trifluoromethyl)imidazo[4,5-c]pyridine.

LCMS:—RT1.28 min, 477.9 (M+H)

1H NMR (400 MHz, CHLOROFORM-d) δ ppm 8.95 (1H, s) 8.10 (1H, s) 7.89 (1H, d) 7.53-7.58 (2H, m) 5.17 (2H, s) 3.75 (3H, s) 3.40 (2H, br d) 3.12 (3H, s) 1.19-1.34 (6H, m)

TABLE P
Examples of compounds of formula (I)

| | | | RT | [M + H] | | |
|---|---|---|---|---|---|---|
| Entry | IUPAC name | STRUCTURE | (min) | (measured) | Method | MP ° C. |
| P1 | 2-[3-ethylsulfonyl-5- (oxetan-3-yloxy)-2-pyridyl]- 3-methyl-6- (trifluoromethyl)imidazo[4,5- c]pyridine | 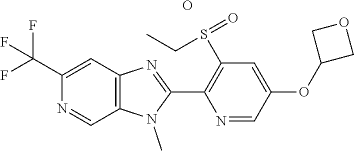 | 1.34 | 443 | 3 | 170-172 |
| P2 | 2-[3-ethylsulfanyl-5- (oxetan-3-yloxy)-2-pyridyl]- 3-methyl-6- (trifluoromethyl)imidazo[4,5- c]pyridine | 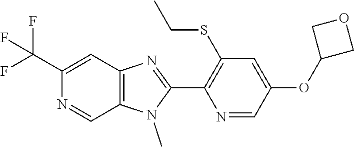 [SMILES: CCSc1cc(OC2COC2)cnc1-c1nc2cc(C(F)(F)F)ncc2n1C] | 0.95 | 411 | 4 | 166-168 |
| P3 | 2-(5-tert-butoxy-3- ethylsulfanyl-2-pyridyl)-3- methyl-6- (trifluoromethyl)imidazo[4,5- c]pyridine | 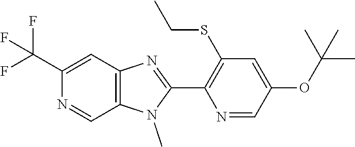 [SMILES: CCSc1cc(OC(C)(C)C)cnc1-c1nc2cc(C(F)(F)F)ncc2n1C] | 1.10 | 411 | 1 | — |
| P4 | 2-[3-ethylsulfonyl-5-(3- methyloxetan-3-yl)oxy-2- pyridyl]-3-methyl-6- (trifluoromethyl)imidazo[4,5- c]pyridine | 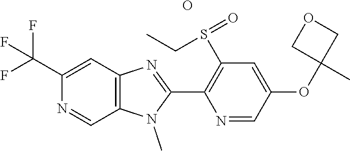 | 0.89 | 457 | 1 | 86-87 |
| P5 | 2-(5-tert-butoxy-3- ethylsulfonyl-2-pyridyl)-3- methyl-6- (trifluoromethyl)imidazo[4,5- c]pyridine | 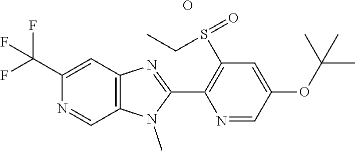 | 1.04 | 443 | 1 | 132-134 |
| P6 | 2-[[5-ethylsulfonyl-6-[3- methyl-6- (trifluoromethyl)imidazo[4,5- c]pyridin-2-yl]-3- pyridyl]oxy]-2,2-difluoro- N,N-dimethyl-acetamide | 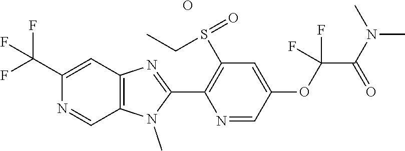 | 1.51 | 508 | 3 | 178-180 |
| P7 | 2-[3-ethylsulfonyl-5-(2- methylsulfonylethoxy)-2- pyridyl]-3-methyl-6- (trifluoromethyl)imidazo[4,5- c]pyridine | 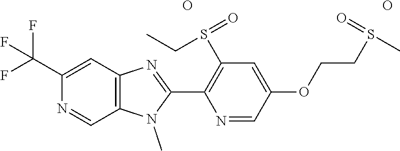 | 1.26 | 493 | 3 | 170-172 |
| P8 | 2-[3-ethylsulfonyl-5-(2- methoxyethoxy)-2-pyridyl]- 3-methyl-6- (trifluoromethyl)imidazo[4,5- c]pyridine | 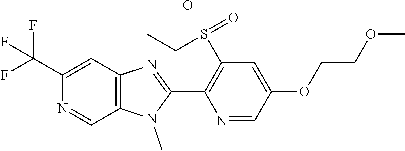 | 0.94 | 445 | 4 | 156-158 |
| P9 | 2-[3-ethylsulfonyl-5- (methylsulfonylmethoxy)-2- pyridyl]-3-methyl-6- (trifluoromethyl)imidazo[4,5- c]pyridine | 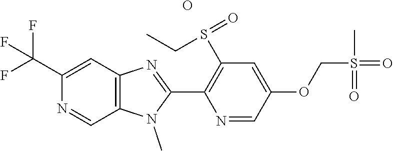 | 1.29 | 479 | 3 | 268-270 |
| P10 | 2-[3-ethylsulfonyl-5- (methylsulfanylmethoxy)-2- pyridyl]-3-methyl-6- (trifluoromethyl)imidazo[4,5- c]pyridine | 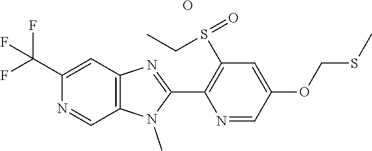 | 1.01 | 447 | 4 | 146-148 |
| P11 | 2-[3-ethylsulfonyl-5-(2- methylsulfanylethoxy)-2- pyridyl]-3-methyl-6- (trifluoromethyl)imidazo[4,5- c]pyridine | 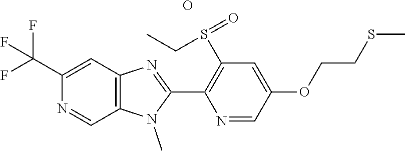 | 1.05 | 461 | 4 | 138-140 |
| P12 | 2-(5-ethoxy-3-ethylsulfonyl- 2-pyridyl)-3-methyl-6- (trifluoromethyl)imidazo[4,5- c]pyridine | 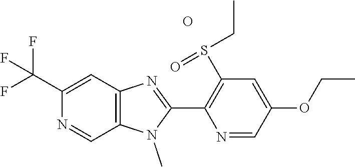 | 0.94 | 415 | 1 | — |
| P13 | 2-(3-ethylsulfonyl-5- methoxy-2-pyridyl)-3- methyl-6- (trifluoromethyl)imidazo[4,5- c]pyridine | 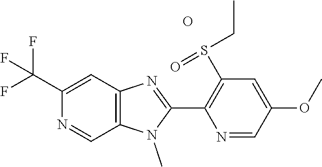 | 0.87 | 401 | 1 | — |
| P14 | 2-[3-ethylsulfanyl-5-(3- methyloxetan-3-yl)oxy-2- pyridyl]-3-methyl-6- (trifluoromethyl)imidazo[4,5- c]pyridine | 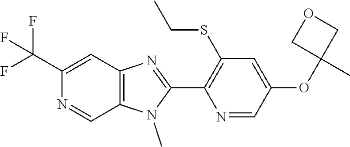 [SMILES: CCSc1cc(OC2(C)COC2)cnc1-c1nc2cc(C(F)(F)F)ncc2n1C] | 0.95 | 425 | 1 | — |
| P15 | 2-[3-ethylsulfonyl-5-(2,2,2- trifluoroethoxy)-2-pyridyl]-3- methyl-6- (trifluoromethyl)imidazo[4,5- c]pyridine | 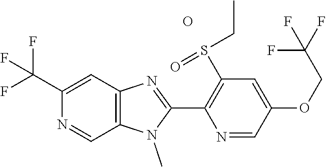 | 0.99 | 469 | 1 | — |
| P16 | 2-[5-(2,2-difluoroethoxy)-3- ethylsulfonyl-2-pyridyl]-3- methyl-6- (trifluoromethyl)imidazo[4,5- c]pyridine | 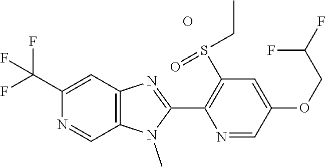 | 1.44 | 451 | 3 | 188-190 |
| P17 | 2-(3-ethylsulfanyl-5-prop-2- ynoxy-2-pyridyl)-3-methyl- 6- (trifluoromethyl)imidazo[4,5- c]pyridine | 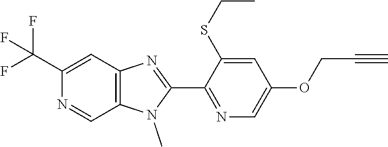 [SMILES: C#CCOc1cnc(-c2nc3cc(C(F)(F)F)ncc3n2C)c(SCC)c1] | — | — | — | 138-140 |
| P18 | 2-[[5-ethylsulfonyl-6-[3- methyl-6- (trifluoromethyl)imidazo[4,5- c]pyridin-2-yl]-3- pyridyl]oxy]-2-methyl- propanenitrile | 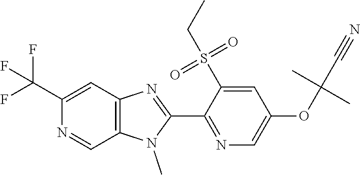 [SMILES: CCS(=O)(=O)c1cc(OC(C)(C)C#N)cnc1-c1nc2cc(C(F)(F)F)ncc2n1C] | 0.97 | 454 | 1 | — |
| P19 | 2-[[5-ethylsulfanyl-6-[3- methyl-6- (trifluoromethyl)imidazo[4,5- c]pyridin-2-yl]-3- pyridyl]oxy]-2-methyl- propanenitrile | 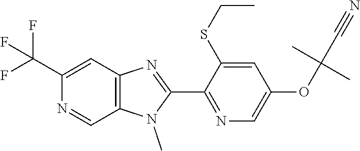 [SMILES: CCSc1cc(OC(C)(C)C#N)cnc1-c1nc2cc(C(F)(F)F)ncc2n1C] | 1.02 | 422 | 1 | — |
| P20 | 2-[[5-ethylsulfonyl-6-[3- methyl-6- (trifluoromethyl)imidazo[4,5- c]pyridin-2-yl]-3- pyridyl]oxy]acetonitrile | 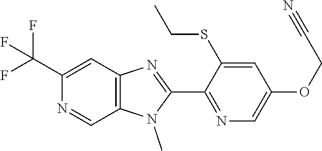 [SMILES: CCSc1cc(OCC#N)cnc1-c1nc2cc(C(F)(F)F)ncc2n1C] | 0.86 | 426 | 1 | — |
| P21 | 2-[[5-ethylsulfanyl-6-[3- methyl-6- (trifluoromethyl)imidazo[4,5- c]pyridin-2-yl]-3- pyridyl]oxy]acetonitrile | 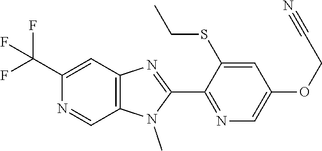 [SMILES: CCSc1cc(OCC#N)cnc1-c1nc2cc(C(F)(F)F)ncc2n1C] | 0.94 | 394 | 1 | — |
| P22 | 2-[[5-ethylsulfonyl-6-[3- methyl-6- (trifluoromethyl)imidazo[4,5- c]pyridin-2-yl]-3- pyridyl]oxy]propanenitrile | 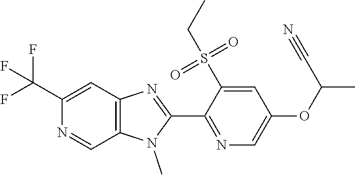 [SMILES: CCS(=O)(=O)c1cc(OC(C)C#N)cnc1-c1nc2cc(C(F)(F)F)ncc2n1C] | 0.92 | 440 | 1 | — |
| P23 | 2-[5-(cyclopropylmethoxy)- 3-ethylsulfanyl-2-pyridyl]-3- methyl-6- (trifluoromethyl)imidazo[4,5- c]pyridine | 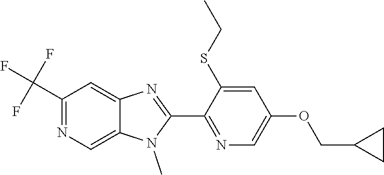 [SMILES: CCSc1cc(OCC2CC2)cnc1-c1nc2cc(C(F)(F)F)ncc2n1C] | 1.05 | 409 | 1 | 135-136 |
| P24 | 2-[5-(cyclopropylmethoxy)- 3-ethylsulfonyl-2-pyridyl]-3- methyl-6- (trifluoromethyl)imidazo[4,5- c]pyridine | 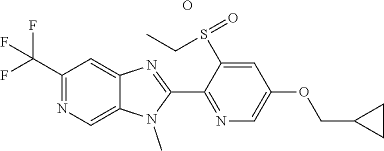 | 0.99 | 441 | 1 | 140-141 |
| P25 | 2-[5-(2,2-dimethylpropoxy)- 3-ethylsulfanyl-2-pyridyl]-3- methyl-6- (trifluoromethyl)imidazo[4,5- c]pyridine | 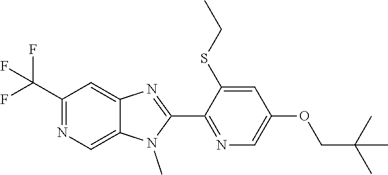 [SMILES: CCSc1cc(OCC(C)(C)C)cnc1-c1nc2cc(C(F)(F)F)ncc2n1C] | 1.17 | 425 | 1 | 82-84 |
| P26 | 2-[5-(2,2-dimethylpropoxy)- 3-ethylsulfonyl-2-pyridyl]-3- methyl-6- (trifluoromethyl)imidazo[4,5- c]pyridine | 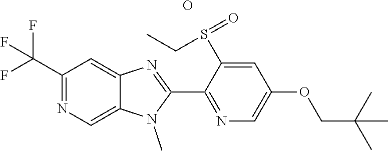 | 1.11 | 457 | 1 | 159-160 |
| P27 | 2-(3-ethylsulfanyl-5- isopropoxy-2-pyridyl)-3- methyl-6- (trifluoromethyl)imidazo[4,5- c]pyridine | 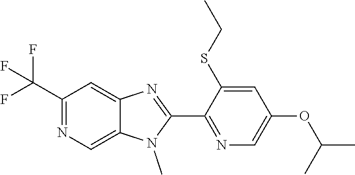 [SMILES: CCSc1cc(OC(C)C)cnc1-c1nc2cc(C(F)(F)F)ncc2n1C] | 1.05 | 397 | 1 | 122-123 |
| P28 | 2-(3-ethylsulfonyl-5- isopropoxy-2-pyridyl)-3- methyl-6- (trifluoromethyl)imidazo[4,5- c]pyridine | 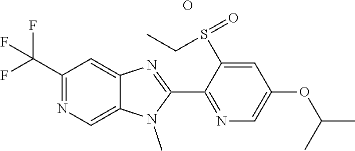 | 0.98 | 429 | 1 | 92-93 |
| P29 | 2-[[5-ethylsulfanyl-6-[3- methyl-6- (trifluoromethyl)imidazo[4,5- c]pyridin-2-yl]-3- pyridyl]oxy]propanenitrile | 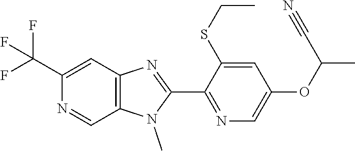 [SMILES: CCSc1cc(OC(C)C#N)cnc1-c1nc2cc(C(F)(F)F)ncc2n1C] | 0.97 | 408 | 1 | — |
| P30 | 2-[3-ethylsulfonyl-5-(2- fluoro-1,1-dimethyl-ethoxy)- 2-pyridyl]-3-methyl-6- (trifluoromethyl)imidazo[4,5- c]pyridine | 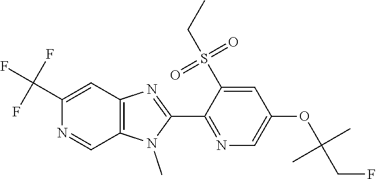 [SMILES: CCS(=O)(=O)c1cc(OC(C)(C)CF)cnc1-c1nc2cc(C(F)(F)F)ncc2n1C] | 0.97 | 461 | 4 | 139-141 |
| P31 | 2-[3-ethylsulfonyl-5-(2- fluoroethoxy)-2-pyridyl]-3- methyl-6- (trifluoromethyl)imidazo[4,5- c]pyridine | 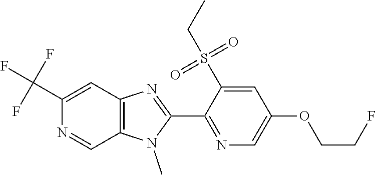 [SMILES: CCS(=O)(=O)c1cc(OCCF)cnc1-c1nc2cc(C(F)(F)F)ncc2n1C] | 0.90 | 433 | 4 | 193-195 |
| P32 | 2-[[5-ethylsulfonyl-6-[3- methyl-6- (trifluoromethyl)imidazo[4,5- c]pyridin-2-yl]-3- pyridyl]oxy]-2- methylsulfanyl-acetonitrile | 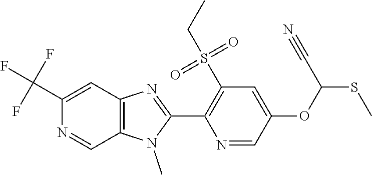 [SMILES: CCS(=O)(=O)c1cc(OC(C#N)SC)cnc1-c1nc2cc(C(F)(F)F)ncc2n1C] | 0.96 | 472 | 4 | 204-206 |
| P33 | methyl 2-[[5-ethylsulfonyl- 6-[3-methyl-6- (trifluoromethyl)imidazo[4,5- c]pyridin-2-yl]-3- pyridyl]oxy]-2- methylsulfonyl-acetate | 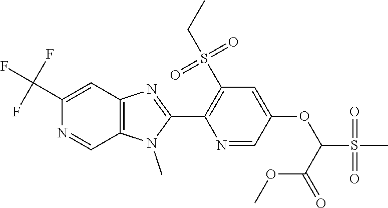 [SMILES: CCS(=O)(=O)c1cc(OC(C(=O)OC)S(C)(=O)=O)cnc1-c1nc2cc(C(F)(F)F)ncc2n1C] | 0.88 | 537 | 4 | 122-124 |
| P34 | methyl 2-[[5-ethylsulfonyl- 6-[3-methyl-6- (trifluoromethyl)imidazo[4,5- c]pyridin-2-yl]-3- pyridyl]oxy]-2- methylsulfanyl-acetate | 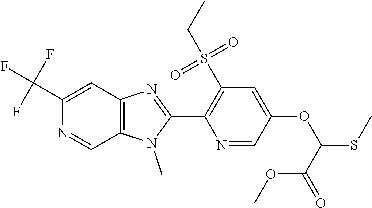 [SMILES: CCS(=O)(=O)c1cc(OC(SC)C(=O)OC)cnc1-c1nc2cc(C(F)(F)F)ncc2n1C] | 0.99 | 505 | 4 | 94-96 |
| P35 | 2-[[5-ethylsulfonyl-6-[3- methyl-6- (trifluoromethyl)imidazo[4,5- c]pyridin-2-yl]-3- pyridyl]oxy]-2- methylsulfanyl-acetamide | 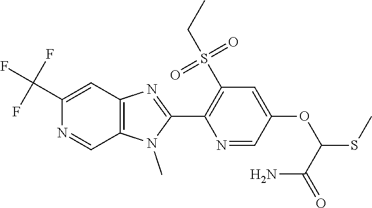 [SMILES: CCS(=O)(=O)c1cc(OC(SC)C(N)=O)cnc1-c1nc2cc(C(F)(F)F)ncc2n1C] | 0.77 | 490 | 4 | 142-144 |
| P36 | 2-[[5-ethylsulfonyl-6-[3- methyl-6- (trifluoromethyl)imidazo[4,5- c]pyridin-2-yl]-3- pyridyl]oxy]-N,N-dimethyl- 2-methylsulfanyl-acetamide | 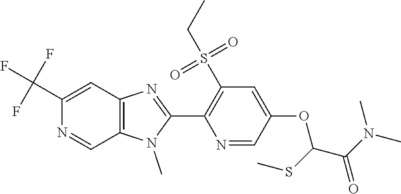 [SMILES: CCS(=O)(=O)c1cc(OC(SC)C(=O)N(C)C)cnc1-c1nc2cc(C(F)(F)F)ncc2n1C] | 0.81 | 550 | 4 | 174-176 |
| P37 | 2-[[5-ethylsulfonyl-6-[3- methyl-6- (trifluoromethyl)imidazo[4,5- c]pyridin-2-yl]-3- pyridyl]oxy]-N-methyl-2- methylsulfanyl-acetamide | 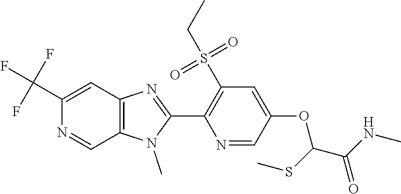 [SMILES: CCS(=O)(=O)c1cc(OC(SC)C(=O)NC)cnc1-c1nc2cc(C(F)(F)F)ncc2n1C] | 0.83 | 504 | 4 | 214-216 |
| P38 | 2-[[5-ethylsulfonyl-6-[3- methyl-6- (trifluoromethyl)imidazo[4,5- c]pyridin-2-yl]-3- pyridyl]oxy]-N,N-dimethyl- 2-methylsulfonyl-acetamide | 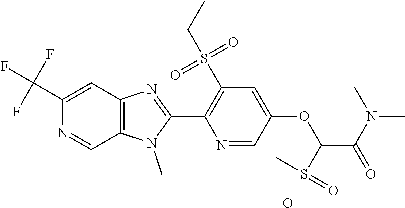 | 0.81 | 550 | 4 | 214-216 |
| P39 | methyl 2-[[5-ethylsulfonyl- 6-[3-methyl-6- (trifluoromethyl)imidazo[4,5- c]pyridin-2-yl]-3- pyridyl]oxy]propanoate | 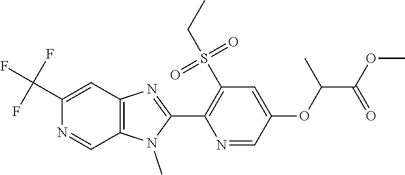 [SMILES: CCS(=O)(=O)c1cc(OC(C)C(=O)OC)cnc1-c1nc2cc(C(F)(F)F)ncc2n1C] | 1.42 | 473 | 3 | 131-133 |
| P40 | 2-[3-ethylsulfonyl-5-(1- fluoroethoxy)-2-pyridyl]-3- methyl-6- (trifluoromethyl)imidazo[4,5- c]pyridine | 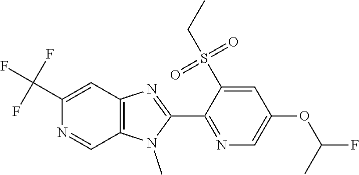 [SMILES: CCS(=O)(=O)c1cc(OC(C)F)cnc1-c1nc2cc(C(F)(F)F)ncc2n1C] | 1.42 | 433 | 3 | 155-157 |
| P41 | 2-[5-(2,2-difluoropropoxy)- 3-ethylsulfonyl-2-pyridyl]-3- methyl-6- (trifluoromethyl)imidazo[4,5- c]pyridine | 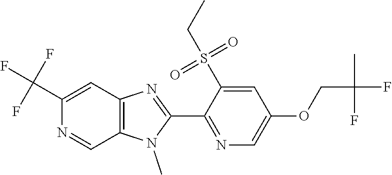 [SMILES: CCS(=O)(=O)c1cc(OCC(C)(F)F)cnc1-c1nc2cc(C(F)(F)F)ncc2n1C] | 1.44 | 465 | 3 | 184-186 |
| P42 | 2-[5-(3,3- difluorocyclobutoxy)-3- ethylsulfonyl-2-pyridyl]-3- methyl-6- (trifluoromethyl)imidazo[4,5- c]pyridine | 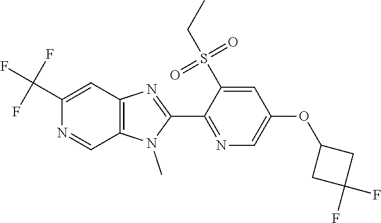 [SMILES: CCS(=O)(=O)c1cc(OC2CC(F)(F)C2)cnc1-c1nc2cc(C(F)(F)F)ncc2n1C] | 1.05 | 477 | 4 | 166-168 |
| P43 | 2-ethyl-2-[[5-ethylsulfanyl- 6-[3-methyl-6- (trifluoromethyl)imidazo[4,5- c]pyridin-2-yl]-3- pyridyl]oxy]butanenitrile | 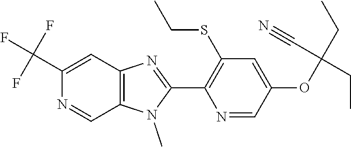 [SMILES: CCSc1cc(OC(C#N)(CC)CC)cnc1-c1nc2cc(C(F)(F)F)ncc2n1C] | 1.17 | 450 | 4 | 86-88 |
| P44 | 2-ethyl-2-[[5-ethylsulfonyl- 6-[3-methyl-6- (trifluoromethyl)imidazo[4,5- c]pyridin-2-yl]-3- pyridyl]oxy]butanenitrile | 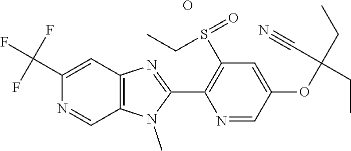 | 1.08 | 482 | 4 | 156-158 |
| P45 | 2-[3-ethylsulfonyl-5-(3- methoxycyclobutoxy)-2- pyridyl]-3-methyl-6- (trifluoromethyl)imidazo[4,5- c]pyridine | 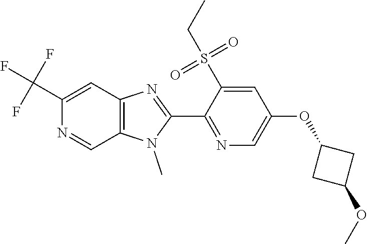 [SMILES: CCS(=O)(=O)c1cc(O[C@H]2C[C@H](OC)C2)cnc1-c1nc2cc(C(F)(F)F)ncc2n1C] | 0.97 | 471 | 4 | 163-165 |
| P46 | 3-[[5-ethylsulfonyl-6-[3- methyl-6- (trifluoromethyl)imidazo[4,5- c]pyridin-2-yl]-3- pyridyl]oxy] cyclobutanecarbonitrile | 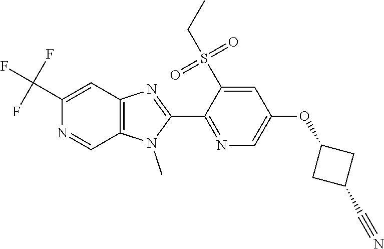 [SMILES: CCS(=O)(=O)c1cc(O[C@H]2C[C@@H](C#N)C2)cnc1-c1nc2cc(C(F)(F)F)ncc2n1C] | 0.94 | 466 | 4 | 217-219 |
| P47 | 3-[[5-ethylsulfonyl-6-[3- methyl-6- (trifluoromethyl)imidazo[4,5- c]pyridin-2-yl]-3- pyridyl]oxy]-3-methyl- butanenitrile | 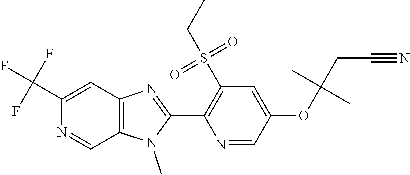 [SMILES: CCS(=O)(=O)c1cc(OC(C)(C)CC#N)cnc1-c1nc2cc(C(F)(F)F)ncc2n1C] | 0.97 | 468 | 4 | 174-176 |
| P48 | 2-[3-ethylsulfonyl-5-(2- methoxy-1,1-dimethyl- ethoxy)-2-pyridyl]-3-methyl- 6- (trifluoromethyl)imidazo[4,5- c]pyridine | 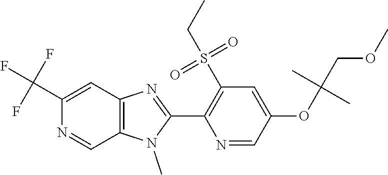 [SMILES: CCS(=O)(=O)c1cc(OC(C)(C)COC)cnc1-c1nc2cc(C(F)(F)F)ncc2n1C] | 0.99 | 473 | 4 | 153-155 |
| P49 | 2-[5-(1,1-dimethyl-2- methylsulfonyl-ethoxy)-3- ethylsulfonyl-2-pyridyl]-3- methyl-6- (trifluoromethyl)imidazo[4,5- c]pyridine | 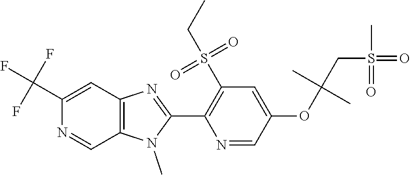 [SMILES: CCS(=O)(=O)c1cc(OC(C)(C)CS(C)(=O)=O)cnc1-c1nc2cc(C(F)(F)F)ncc2n1C] | 0.94 | 521 | 4 | 92-94 |
| P50 | 2-[5-(1,1-dimethyl-2- methylsulfanyl-ethoxy)-3- ethylsulfonyl-2-pyridyl]-3- methyl-6- (trifluoromethyl)imidazo[4,5- c]pyridine | 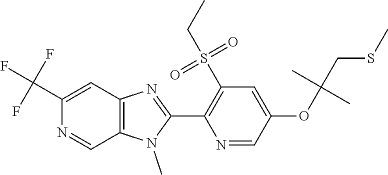 [SMILES: CCS(=O)(=O)c1cc(OC(C)(C)CSC)cnc1-c1nc2cc(C(F)(F)F)ncc2n1C] | 1.1 | 489 | 4 | 131-133 |
| P51 | 2-[[5-ethylsulfonyl-6-[3- methyl-6- (trifluoromethyl)imidazo[4,5- c]pyridin-2-yl]-3- pyridyl]oxy]-N,N-dimethyl- acetamide | 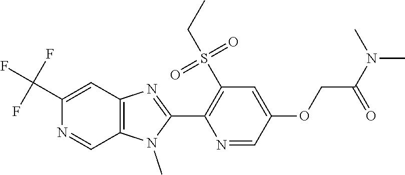 [SMILES: CCS(=O)(=O)c1cc(OCC(=O)N(C)C)cnc1-c1nc2cc(C(F)(F)F)ncc2n1C] | 0.85 | 472 | 4 | 210-212 |
| P52 | 2-[3-ethylsulfonyl-5-(3- methylsulfonylpropoxy)-2- pyridyl]-3-methyl-6- (trifluoromethyl)imidazo[4,5- c]pyridine | 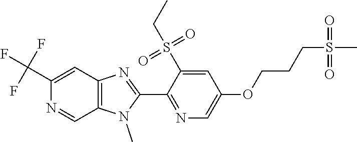 [SMILES: CCS(=O)(=O)c1cc(OCCCS(C)(=O)=O)cnc1-c1nc2cc(C(F)(F)F)ncc2n1C] | 0.85 | 507 | 4 | 112-114 |
| P53 | 2-[3-ethylsulfonyl-5-[2- fluoro-1- (fluoromethyl)ethoxy]-2- pyridyl]-3-methyl-6- (trifluoromethyl)imidazo[4,5- c]pyridine | 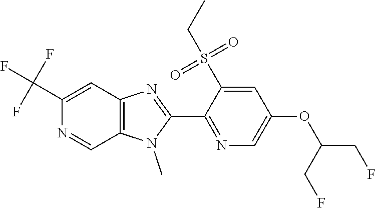 [SMILES: CCS(=O)(=O)c1cc(OC(CF)CF)cnc1-c1nc2cc(C(F)(F)F)ncc2n1C] | 0.95 | 465 | 4 | 134-136 |
| P54 | 2-[3-ethylsulfonyl-5-[(3- methyloxetan-3- yl)methoxy]-2-pyridyl]-3- methyl-6- (trifluoromethyl)imidazo[4,5- c]pyridine | 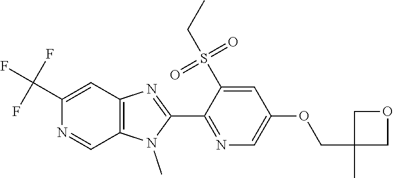 [SMILES: CCS(=O)(=O)c1cc(OCC2(C)COC2)cnc1-c1nc2cc(C(F)(F)F)ncc2n1C] | 0.92 | 471 | 4 | 180-182 |
| P55 | 2-[[5-ethylsulfonyl-6-[3- methyl-6- (trifluoromethyl)imidazo[4,5- c]pyridin-2-yl]-3- pyridyl]oxy]-N,2-dimethyl- propanamide | 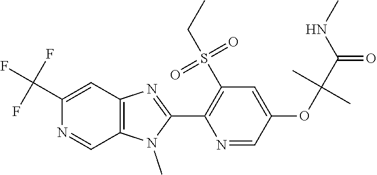 [SMILES: CCS(=O)(=O)c1cc(OC(C)(C)C(=O)NC)cnc1-c1nc2cc(C(F)(F)F)ncc2n1C] | 0.87 | 486 | 4 | 170-172 |
| P56 | 2-[3-ethylsulfonyl-5-(3- methylsulfanylpropoxy)-2- pyridyl]-3-methyl-6- (trifluoromethyl)imidazo[4,5- c]pyridine | 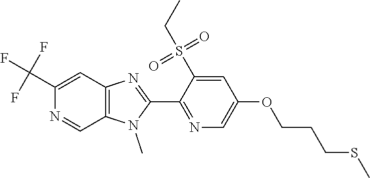 [SMILES: CCS(=O)(=O)c1cc(OCCCSC)cnc1-c1nc2cc(C(F)(F)F)ncc2n1C] | 1.04 | 475 | 4 | 108-110 |
| P57 | ethyl 2-[[5-ethylsulfonyl-6- [3-methyl-6- (trifluoromethyl)imidazo[4,5- c]pyridin-2-yl]-3- pyridyl]oxy]-2-methyl- propanoate | 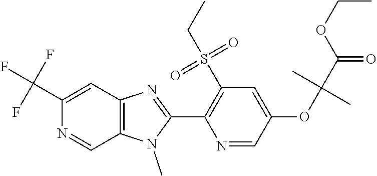 [SMILES: CCOC(=O)C(C)(C)Oc1cnc(-c2nc3cc(C(F)(F)F)ncc3n2C)c(S(=O)(=O)CC)c1] | 1.14 | 501 | 4 | 145-147 |
| P58 | 2-[[5-ethylsulfonyl-6-[3- methyl-6- (trifluoromethyl)imidazo[4,5- c]pyridin-2-yl]-3- pyridyl]oxy]-2-methyl- propanoic acid | 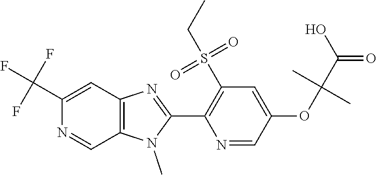 [SMILES: CCS(=O)(=O)c1cc(OC(C)(C)C(=O)O)cnc1-c1nc2cc(C(F)(F)F)ncc2n1C] | 0.88 | 473 | 4 | 242-244 |
| P59 | 2-[[5-ethylsulfonyl-6-[3- methyl-6- (trifluoromethyl)imidazo[4,5- c]pyridin-2-yl]-3- pyridyl]oxy]-N,N,2- trimethyl-propanamide | 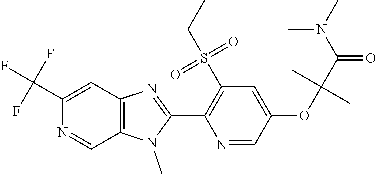 [SMILES: CCS(=O)(=O)c1cc(OC(C)(C)C(=O)N(C)C)cnc1-c1nc2cc(C(F)(F)F)ncc2n1C] | 0.93 | 500 | 4 | 185-187 |
| P60 | 2-(3-ethylsulfonyl-5- isopropoxy-2-pyridyl)-3- methyl-6- (trifluoromethylsulfinyl) imidazo[4,5-c]pyridine | 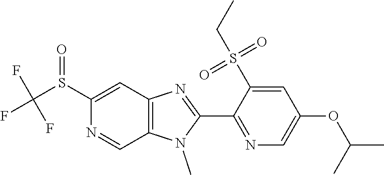 [SMILES: CCS(=O)(=O)c1cc(OC(C)C)cnc1-c1nc2cc(S(=O)C(F)(F)F)ncc2n1C] | 0.96 | 477 | 1 | — |
| P61 | [5-ethylsulfonyl-6-[3- methyl-6- (trifluoromethyl)imidazo[4,5- c]pyridin-2-yl]-3- pyridyl]oxymethyl-trimethyl- silane | 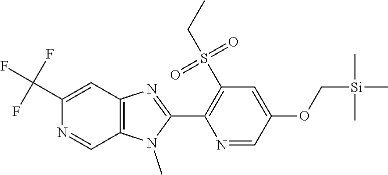 [SMILES: CCS(=O)(=O)c1cc(OC[Si](C)(C)C)cnc1-c1nc2cc(C(F)(F)F)ncc2n1C] | 1.15 | 473 | 4 | 86-88 |
| P62 | 2-[5-(cyclobutoxy)-3- ethylsulfonyl-2-pyridyl]-3- methyl-6- (trifluoromethyl)imidazo[4,5- c]pyridine | 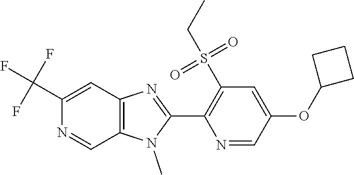 [SMILES: CCS(=O)(=O)c1cc(OC2CCC2)cnc1-c1nc2cc(C(F)(F)F)ncc2n1C] | 1.07 | 441 | 4 | 146-148 |
| P63 | (E)-3-[[5-ethylsulfonyl-6-[3- methyl-6- (trifluoromethyl)imidazo[4,5- c]pyridin-2-yl]-3- pyridyl]oxy]-N-methoxy-3- methyl-butan-2-imine | 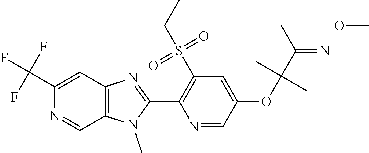 | 1.14 | 500 | 4 | 210-212 |
| P64 | 1-[[5-ethylsulfonyl-6-[3- methyl-6- (trifluoromethyl)imidazo[4,5- c]pyridin-2-yl]-3- pyridyl]oxy]-N-methoxy- propan-2-imine | 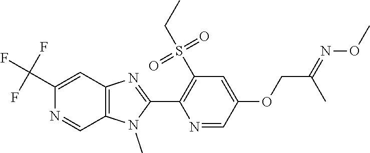 [SMILES: CCS(=O)(=O)c1cc(OC/C(C)=N/OC)cnc1-c1nc2cc(C(F)(F)F)ncc2n1C] | 1.03 | 472 | 4 | 137-139 |
| P65 | 1-[[5-ethylsulfonyl-6-[3- methyl-6- (trifluoromethyl)imidazo[4,5- c]pyridin-2-yl]-3- pyridyl]oxy]propan-2-one | 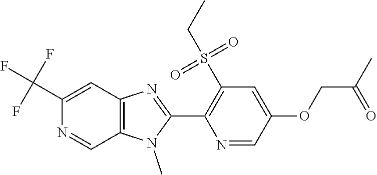 [SMILES: CCS(=O)(=O)c1cc(OCC(C)=O)cnc1-c1nc2cc(C(F)(F)F)ncc2n1C] | 0.91 | 443 | 4 | 184-186 |
| P66 | 3-[[5-ethylsulfonyl-6-[3- methyl-6- (trifluoromethyl)imidazo[4,5- c]pyridin-2-yl]-3- pyridyl]oxy]-3-methyl- butan-2-one | 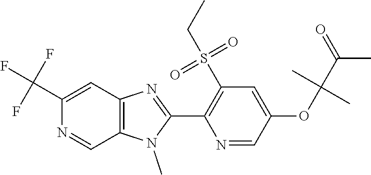 [SMILES: CCS(=O)(=O)c1cc(OC(C)(C)C(C)=O)cnc1-c1nc2cc(C(F)(F)F)ncc2n1C] | 1.05 | 471 | 4 | 138-140 |
| P67 | 2-[5-[(2,2- difluorocyclopropyl) methoxy]- 3-ethylsulfonyl-2-pyridyl]- 3-methyl-6- (trifluoromethyl)imidazo[4,5- c]pyridine | 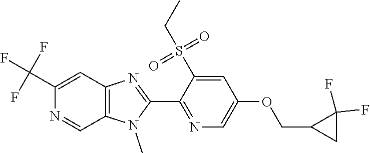 [SMILES: CCS(=O)(=O)c1cc(OCC2CC2(F)F)cnc1-c1nc2cc(C(F)(F)F)ncc2n1C] | 1.41 | 477 | 3 | 186-188 |
| P68 | 2-[3-ethylsulfonyl-5- (methoxymethoxy)-2- pyridyl]-3-methyl-6- (trifluoromethyl)imidazo[4,5- c]pyridine | 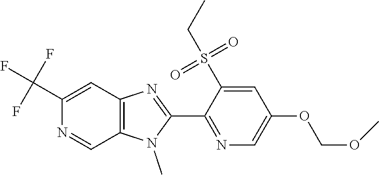 [SMILES: CCS(=O)(=O)c1cc(OCOC)cnc1-c1nc2cc(C(F)(F)F)ncc2n1C] | 0.97 | 431 | 4 | 134-136 |
| P69 | 2-[5-(cyclobutylmethoxy)-3- ethylsulfonyl-2-pyridyl]-3- methyl-6- (trifluoromethyl)imidazo[4,5- c]pyridine | 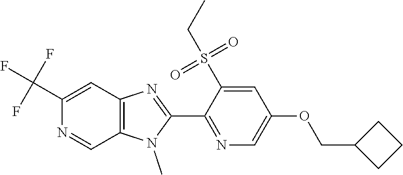 [SMILES: CCS(=O)(=O)c1cc(OCC2CCC2)cnc1-c1nc2cc(C(F)(F)F)ncc2n1C] | 1.13 | 455 | 4 | 150-152 |
| P70 | 4-[[5-ethylsulfonyl-6-[3- methyl-6- (trifluoromethyl)imidazo[4,5- c]pyridin-2-yl]-3- pyridyl]oxy]butanenitrile | 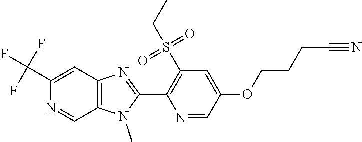 [SMILES: CCS(=O)(=O)c1cc(OCCCC#N)cnc1-c1nc2cc(C(F)(F)F)ncc2n1C] | 0.89 | 454 | 4 | 182-184 |
| P71 | 2-(3-ethylsulfonyl-5- isopropoxy-2-pyridyl)-3- methyl-6- (trifluoromethylsulfanyl) imidazo[4,5-c]pyridine | 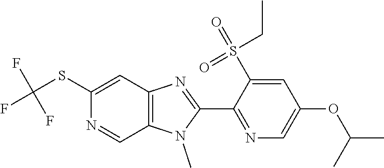 [SMILES: CCS(=O)(=O)c1cc(OC(C)C)cnc1-c1nc2cc(SC(F)(F)F)ncc2n1C] | 1.04 | 461 | 1 | |
| P72 | 2-(3-ethylsulfonyl-5- isopropoxy-2-pyridyl)-3- methyl-6- (trifluoromethylsulfonyl) imidazo[4,5-c]pyridine | 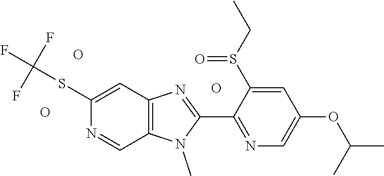 | 1.03 | 493 | 1 | 167-168 |
| P73 | 2-(3-ethylsulfanyl-5- isopropoxy-2-pyridyl)-3- methyl-6- (trifluoromethylsulfonyl) imidazo[4,5-c]pyridine | 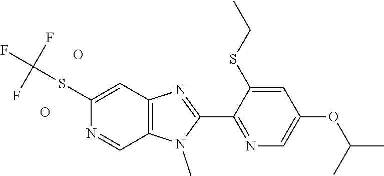 | 1.11 | 461 | 1 | 122-123 |
| P74 | 2-(3-ethylsulfanyl-5- isopropoxy-2-pyridyl)-3- methyl-6- (trifluoromethylsulfanyl) imidazo[4,5-c]pyridine | 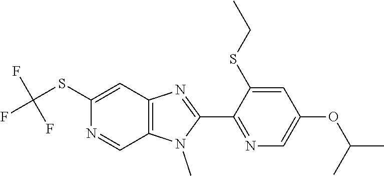 [SMILES: CCSc1cc(OC(C)C)cnc1-c1nc2cc(SC(F)(F)F)ncc2n1C] | 1.11 | 429 | 1 | 113.5-114.3 |
| P75 | 2-(3-ethylsulfonyl-5- isobutoxy-2-pyridyl)-3- methyl-6- (trifluoromethyl)imidazo[4,5- c]pyridine | 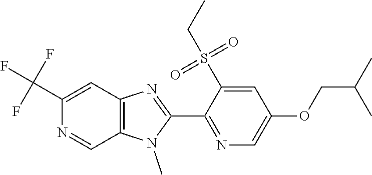 [SMILES: CCS(=O)(=O)c1cc(OCC(C)C)cnc1-c1nc2cc(C(F)(F)F)ncc2n1C] | 1.07 | 443 | 1 | 149-150 |
| P76 | 2-[3-ethylsulfonyl-5-(2,2,2- trifluoro-1,1-dimethyl- ethoxy)-2-pyridyl]-3-methyl- 6- (trifluoromethyl)imidazo[4,5- c]pyridine | 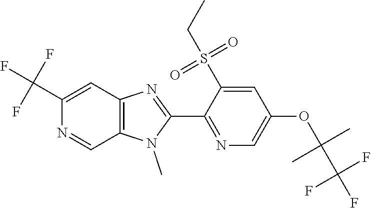 [SMILES: CCS(=O)(=O)c1cc(OC(C)(C)C(F)(F)F)cnc1-c1nc2cc(C(F)(F)F)ncc2n1C] | 1.09 | 497 | 4 | 86-88 |
| P77 | 2-[[5-ethylsulfanyl-6-[3- methyl-6- (trifluoromethyl)imidazo[4,5- c]pyridin-2-yl]-3- pyridyl]oxy]-2-methyl- propanamide | 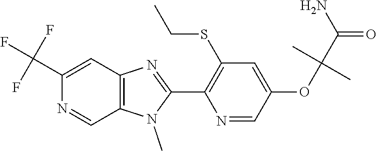 [SMILES: CCSc1cc(OC(C)(C)C(N)=O)cnc1-c1nc2cc(C(F)(F)F)ncc2n1C] | 0.85 | 440 | 1 | — |
| P78 | 2-[5-(1,1-difluoroethoxy)-3- ethylsulfonyl-2-pyridyl]-3- methyl-6- (trifluoromethyl)imidazo[4,5- c]pyridine | 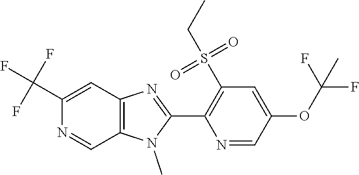 [SMILES: CCS(=O)(=O)c1cc(OC(C)(F)F)cnc1-c1nc2cc(C(F)(F)F)ncc2n1C] | 1.04 | 451 | 4 | 196-198 |
| P79 | 1-[[5-ethylsulfonyl-6-[3- methyl-6- (trifluoromethyl)imidazo[4,5- c]pyridin-2-yl]-3- pyridyl]oxymethyl] cyclopropanecarbonitrile | 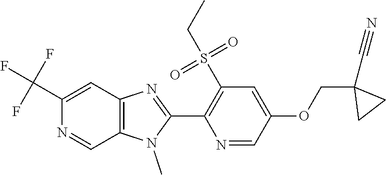 [SMILES: CCS(=O)(=O)c1cc(OCC2(C#N)CC2)cnc1-c1nc2cc(C(F)(F)F)ncc2n1C] | 0.92 | 466 | 4 | 190-192 |
| P80 | [5-ethylsulfonyl-6-[3- methyl-6- (trifluoromethyl)imidazo[4,5- c]pyridin-2-yl]-3- pyridyl]oxymethyl-imino- methyl-oxo-$\|\hat{ }{6}-sulfane | 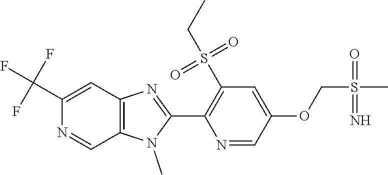 [SMILES: CCS(=O)(=O)c1cc(OCS(C)(=N)=O)cnc1-c1nc2cc(C(F)(F)F)ncc2n1C] | 0.77 | 478 | 4 | 202-204 |
| P81 | 2-[5-(2,2-difluoroethoxy)-3- ethylsulfanyl-2-pyridyl]-3- methyl-6- (trifluoromethyl)imidazo[4,5- c]pyridine | 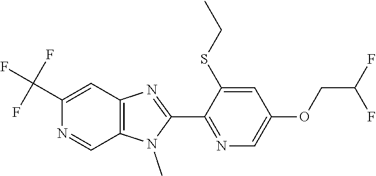 [SMILES: CCSc1cc(OCC(F)F)cnc1-c1nc2cc(C(F)(F)F)ncc2n1C] | 0.98 | 419 | 1 | |
| P82 | 2-[5-(1,1-dimethylprop-2- ynoxy)-3-ethylsulfanyl-2- pyridyl]-3-methyl-6- (trifluoromethyl)imidazo[4,5- c]pyridine | 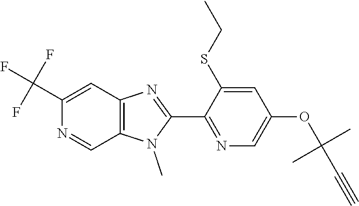 [SMILES: C#CC(C)(C)Oc1cnc(-c2nc3cc(C(F)(F)F)ncc3n2C)c(SCC)c1] | 1.01 | 421 | 1 | 89-91 |
| P83 | 3-cyclopropyl-5-[[5- ethylsulfonyl-6-[3-methyl-6- (trifluoromethyl)imidazo[4,5- c]pyridin-2-yl]-3- pyridyl]oxymethyl]isoxazole | 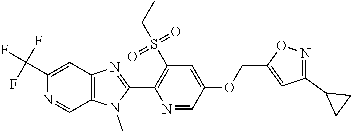 [SMILES: CCS(=O)(=O)c1cc(OCc2cc(C3CC3)no2)cnc1-c1nc2cc(C(F)(F)F)ncc2n1C] | 1.03 | 508 | 4 | 198-200 |
| P84 | ethyl [5-ethylsulfonyl-6-[3- methyl-6- (trifluoromethyl)imidazo[4,5- c]pyridin-2-yl]-3-pyridyl] carbonate | 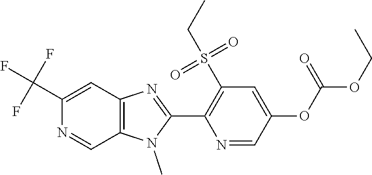 [SMILES: CCOC(=O)Oc1cnc(-c2nc3cc(C(F)(F)F)ncc3n2C)c(S(=O)(=O)CC)c1] | 1.02 | 459 | 4 | 174-176 |
| P85 | [5-ethylsulfonyl-6-[3- methyl-6- (trifluoromethyl)imidazo[4,5- c]pyridin-2-yl]-3-pyridyl] N,N-dimethylcarbamate | 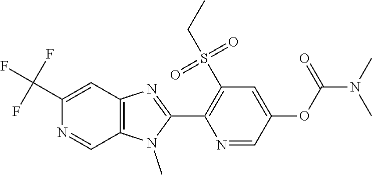 [SMILES: CCS(=O)(=O)c1cc(OC(=O)N(C)C)cnc1-c1nc2cc(C(F)(F)F)ncc2n1C] | 1.42 | 458 | 3 | 186-188 |
| P86 | 6-(difluoromethylsulfanyl)- 2-(3-ethylsulfonyl-5- isopropoxy-2-pyridyl)-3- methyl-imidazo[4,5- c]pyridine | 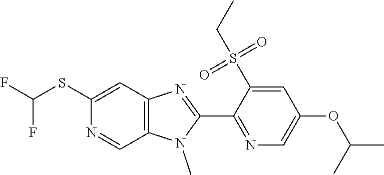 [SMILES: CCS(=O)(=O)c1cc(OC(C)C)cnc1-c1nc2cc(SC(F)F)ncc2n1C] | 1.00 | 444 | 1 | 149-151 |
| P87 | 6-(difluoromethylsulfinyl)-2- (3-ethylsulfonyl-5- isopropoxy-2-pyridyl)-3- methyl-imidazo[4,5- c]pyridine | 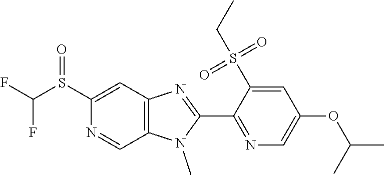 [SMILES: CCS(=O)(=O)c1cc(OC(C)C)cnc1-c1nc2cc(S(=O)C(F)F)ncc2n1C] | 0.88 | 360 | 1 | 203-205 |
| P88 | 6-(difluoromethylsulfonyl)- 2-(3-ethylsulfonyl-5- isopropoxy-2-pyridyl)-3- methyl-imidazo[4,5- c]pyridine | 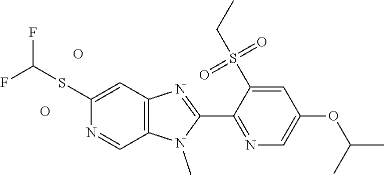 | 0.95 | 476 | 1 | 165-167 |
| P89 | 2-[[5-ethylsulfonyl-6-[3- methyl-6- (trifluoromethyl)imidazo[4,5- c]pyridin-2-yl]-3- pyridyl]oxy]-2-methyl- propanamide | 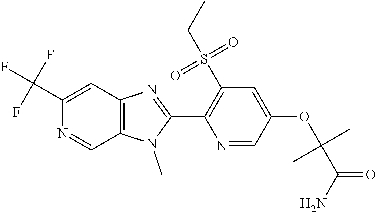 [SMILES: CCS(=O)(=O)c1cc(OC(C)(C)C(N)=O)cnc1-c1nc2cc(C(F)(F)F)ncc2n1C] | 0.81 | 472 | 1 | 177-179 |
| P90 | [5-ethylsulfonyl-6-[3- methyl-6- (trifluoromethyl)imidazo[4,5- c]pyridin-2-yl]-3-pyridyl] fluoromethanesulfonate | 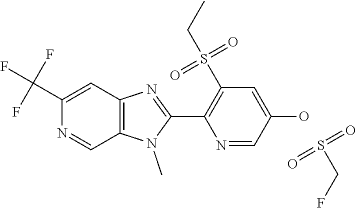 | 0.95 | 484 | 1 | 182-185 |
| P91 | [5-ethylsulfonyl-6-[3- methyl-6- (trifluoromethyl)imidazo[4,5- c]pyridin-2-yl]-3-pyridyl] difluoromethanesulfonate | 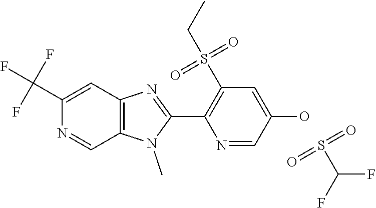 | 1.04 | 501 | 1 | 178.5-182 |
| P92 | [5-ethylsulfonyl-6-[3- methyl-6- (trifluoromethyl)imidazo[4,5- c]pyridin-2-yl]-3-pyridyl] trifluoromethanesulfonate | 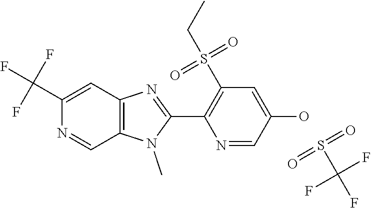 | 1.07 | 519 | 1 | 79-82 |
| P93 | [5-ethylsulfonyl-6-[3- methyl-6- (trifluoromethyl)imidazo[4,5- c]pyridin-2-yl]-3-pyridyl] methanesulfonate | 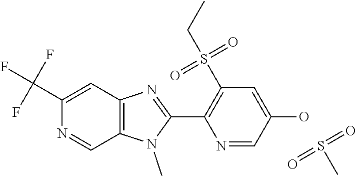 | 0.89 | 465 | 1 | — |
| P94 | 2-(5-benzyloxy-3- ethylsulfonyl-2-pyridyl)-3- methyl-6- (trifluoromethyl)imidazo[4,5- c]pyridine | 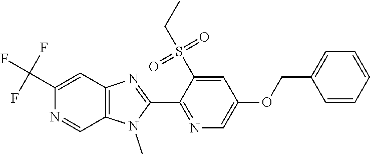 [SMILES: CCS(=O)(=O)c1cc(OCc2ccccc2)cnc1-c1nc2cc(C(F)(F)F)ncc2n1C] | 1.52 | 477 | 3 | |
| P95 | 2-(5-allyloxy-3- ethylsulfanyl-2-pyridyl)-3- methyl-6- (trifluoromethyl)imidazo[4,5- c]pyridine | 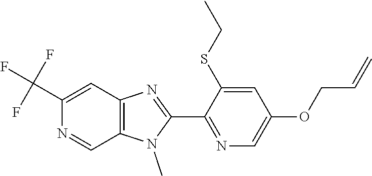 [SMILES: C=CCOc1cnc(-c2nc3cc(C(F)(F)F)ncc3n2C)c(SCC)c1] | 1.08 | 395 | 1 | 128-129 |
| P96 | (E)-1-(3,5-difluorophenyl)- N-[[5-ethylsulfonyl-6-[3- methyl-6- (trifluoromethyl)imidazo[4,5- c]pyridin-2-yl]-3- pyridyl]oxy]ethanimine | 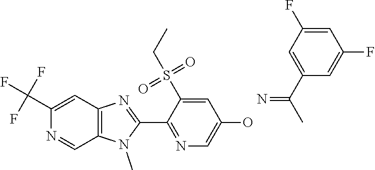 | 1.23 | 540 | 1 | 210-212 |
| P97 | N-[[5-ethylsulfonyl-6-[3- methyl-6- (trifluoromethyl)imidazo[4,5- c]pyridin-2-yl]-3- pyridyl]oxy]propan-2-imine | 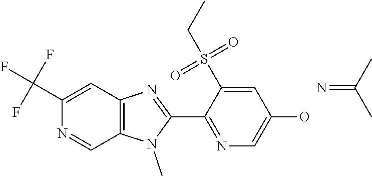 | 1.10 | 442 | 1 | 181-182 |
| P98 | 2-[3-ethylsulfonyl-5- (trifluoromethoxy)-2- pyridyl]-3-methyl-6- (trifluoromethyl)imidazo[4,5- c]pyridine | 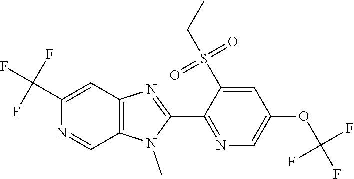 [SMILES: CCS(=O)(=O)c1cc(OC(F)(F)F)cnc1-c1nc2cc(C(F)(F)F)ncc2n1C] | 1.02 | 455 | 1 | 163-164 |
| P99 | 2-[5- [bromo(difluoro)methoxy]- 3-ethylsulfonyl-2-pyridyl]-3- methyl-6- (trifluoromethyl)imidazo[4,5- c]pyridine | 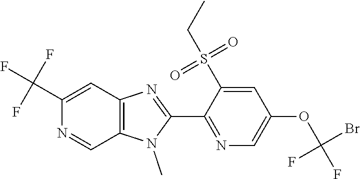 [SMILES: CCS(=O)(=O)c1cc(OC(F)(F)Br)cnc1-c1nc2cc(C(F)(F)F)ncc2n1C] | 1.02 | 515/517 | 1 | 184-186 |
| P100 | 2-[2-ethylsulfonyl-4-[2- fluoro-1- (fluoromethyl)ethoxy]phenyl]- 3-methyl-6- (trifluoromethyl)imidazo[4,5- c]pyridine | 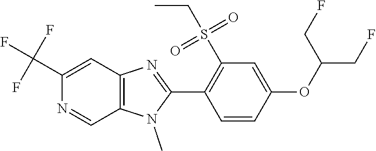 [SMILES: CCS(=O)(=O)c1cc(OC(CF)CF)ccc1-c1nc2cc(C(F)(F)F)ncc2n1C] | 1.41 | 464 | 3 | 155-157 |
| P101 | 1-[[3-ethylsulfonyl-4-[3- methyl-6- (trifluoromethyl)imidazo[4,5- c]pyridin-2- yl]phenoxy]methyl] cyclopropanecarbonitrile | 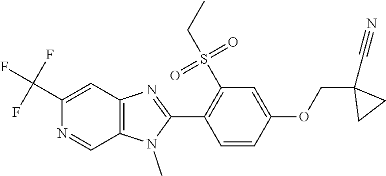 [SMILES: CCS(=O)(=O)c1cc(OCC2(C#N)CC2)ccc1-c1nc2cc(C(F)(F)F)ncc2n1C] | 1.38 | 465 | 3 | 214-216 |
| P102 | 2-[2-ethylsulfonyl-4-(2- methylsulfonylethoxy) phenyl]-3-methyl-6- (trifluoromethyl)imidazo[4,5- c]pyridine | 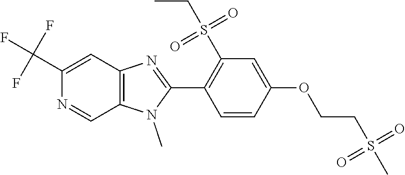 [SMILES: CCS(=O)(=O)c1cc(OCCS(C)(=O)=O)ccc1-c1nc2cc(C(F)(F)F)ncc2n1C] | 0.83 | 492 | 4 | 226-228 |
| P103 | 2-[3-ethylsulfonyl-4-[3- methyl-6- (trifluoromethyl)imidazo[4,5- c]pyridin-2-yl]phenoxy]-2- methyl-propanenitrile | 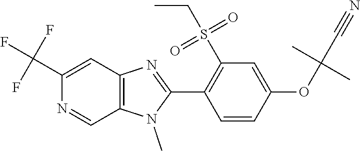 [SMILES: CCS(=O)(=O)c1cc(OC(C)(C)C#N)ccc1-c1nc2cc(C(F)(F)F)ncc2n1C] | 1.44 | 453 | 3 | 192-194 |
| P104 | 2-[2-ethylsulfonyl-4-(2- methylsulfanylethoxy) phenyl]-3-methyl-6- (trifluoromethyl)imidazo[4,5- c]pyridine | 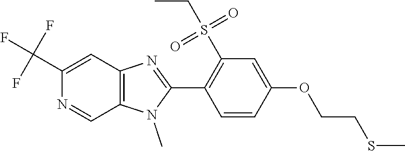 [SMILES: CCS(=O)(=O)c1cc(OCCSC)ccc1-c1nc2cc(C(F)(F)F)ncc2n1C] | 1.41 | 460 | 3 | 168-170 |
| P105 | 2-[2-ethylsulfonyl-4- (methylsulfonylmethoxy) phenyl]-3-methyl-6- (trifluoromethyl)imidazo[4,5- c]pyridine | 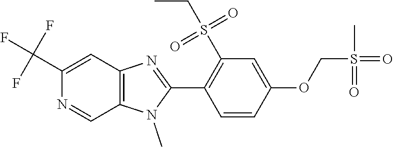 [SMILES: CCS(=O)(=O)c1cc(OCS(C)(=O)=O)ccc1-c1nc2cc(C(F)(F)F)ncc2n1C] | 1.28 | 478 | 3 | 214-216 |
| P106 | 2-[2-ethylsulfonyl-4- (methylsulfanylmethoxy) phenyl]-3-methyl-6- (trifluoromethyl)imidazo[4,5- c]pyridine | 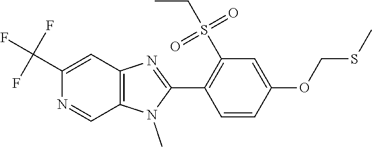 [SMILES: CCS(=O)(=O)c1cc(OCSC)ccc1-c1nc2cc(C(F)(F)F)ncc2n1C] | 1.41 | 456 | 3 | 163-165 |
| P107 | 2-[4-(2,2-difluoroethoxy)-2- ethylsulfonyl-phenyl]-3- methyl-6- (trifluoromethyl)imidazo[4,5- c]pyridine | 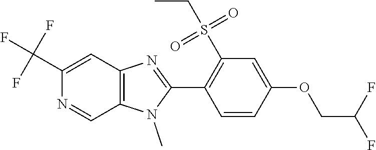 [SMILES: CCS(=O)(=O)c1cc(OCC(F)F)ccc1-c1nc2cc(C(F)(F)F)ncc2n1C] | 1.36 | 450 | 3 | 179-181 |
| P108 | 2-(2-ethylsulfonyl-4- isopropoxy-phenyl)-3- methyl-6- (trifluoromethyl)imidazo[4,5- c]pyridine | 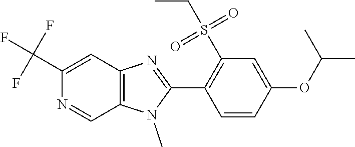 [SMILES: CCS(=O)(=O)c1cc(OC(C)C)ccc1-c1nc2cc(C(F)(F)F)ncc2n1C] | 1.43 | 428 | 3 | 164-166 |
| P109 | 2-[5-(2-bromo-1,1-difluoro- ethoxy)-3-ethylsulfonyl-2- pyridyl]-3-methyl-6- (trifluoromethyl)imidazo[4,5- c]pyridine | 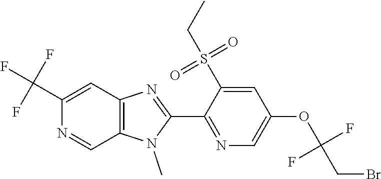 [SMILES: CCS(=O)(=O)c1cc(OC(F)(F)CBr)cnc1-c1nc2cc(C(F)(F)F)ncc2n1C] | 1.50 | 529 | 3 | 162-166 |
| P110 | 2-[[5-ethylsulfanyl-6-[3- methyl-6- (trifluoromethyl)imidazo[4,5- c]pyridin-2-yl]-3- pyridyl]oxy]-2-methyl- propanoic acid | 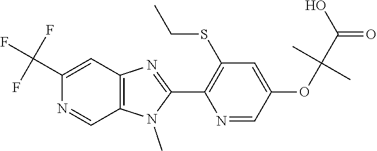 [SMILES: CCSc1cc(OC(C)(C)C(=O)O)cnc1-c1nc2cc(C(F)(F)F)ncc2n1C] | 0.96 | 441 | 4 | 161-163 |
| P111 | 2-[4-(2-bromo-1,1-difluoro- ethoxy)-2-ethylsulfonyl- phenyl]-3-methyl-6- (trifluoromethyl)imidazo[4,5- c]pyridine | 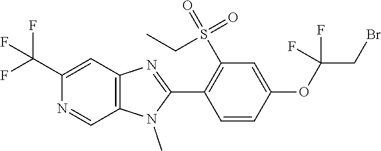 [SMILES: CCS(=O)(=O)c1cc(OC(F)(F)CBr)ccc1-c1nc2cc(C(F)(F)F)ncc2n1C] | 1.49 | 528 | 3 | 137-139 |
| P112 | 2-[4-(1,1-difluoroethoxy)-2- ethylsulfonyl-phenyl]-3- methyl-6- (trifluoromethyl)imidazo[4,5- c]pyridine | 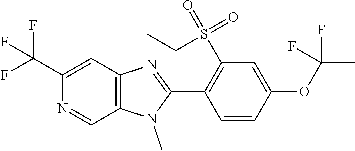 [SMILES: CCS(=O)(=O)c1cc(OC(C)(F)F)ccc1-c1nc2cc(C(F)(F)F)ncc2n1C] | 1.46 | 450 | 3 | 142-144 |

TABLE I
Examples of intermediate compounds of formula IV:

| | | | RT | [M + H] | | MP |
|---|---|---|---|---|---|---|
| Entry | IUPAC name | STRUCTURE | (min) | (measured) | Method | ° C. |
| I1 | 5-ethylsulfonyl-6-[3- methyl-6- (trifluoromethyl)imidazo [4,5-c]pyridin-2- yl]pyridin-3-ol | 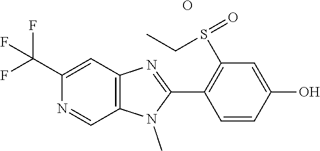 | 0.77 | 387 | 1 | — |
| I2 | 5-ethylsulfanyl-6-[3- methyl-6- (trifluoromethylsulfonyl) imidazo[4,5-c]pyridin-2- yl]pyridin-3-ol | 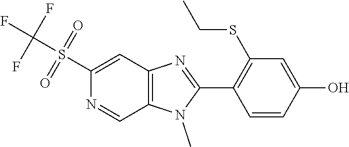 [SMILES: CCSc1cc(O)ccc1-c1nc2cc(S(=O)(=O)C(F)(F)F)ncc2n1C] | 0.89 | 419 | 1 | 267- 270° C. |
| I3 | 5-ethylsulfanyl-6-[3- methyl-6- (trifluoromethylsulfanyl) imidazo[4,5-c]pyridin-2- yl]pyridin-3-ol | 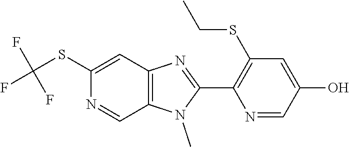 [SMILES: CCSc1cc(O)cnc1-c1nc2cc(SC(F)(F)F)ncc2n1C] | 0.88 | 387 | 1 | 218- 219° C. |
| I4 | 2-[[5-ethylsulfonyl-6-[3- methyl-6- (trifluoromethyl)imidazo [4,5-c]pyridin-2-yl]-3- pyridyl]oxy]-2-methyl- propan-1-ol | 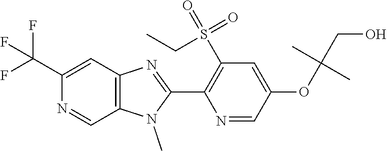 [SMILES: CCS(=O)(=O)c1cc(OC(C)(C)CO)cnc1-c1nc2cc(C(F)(F)F)ncc2n1C] | 0.81 | 459 | 1 | — |
| I5 | [2-[[5-ethylsulfonyl-6-[3- methyl-6- (trifluoromethyl)imidazo [4,5-c]pyridin-2-yl]-3- pyridyl]oxy]-2-methyl- propyl] trifluoromethanesulfonate | 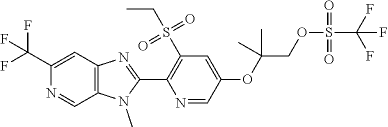 [SMILES: CCS(=O)(=O)c1cc(OC(C)(C)COS(=O)(=O)C(F)(F)F)cnc1-c1nc2cc(C(F)(F)F)ncc2n1C] | 1.07 | 591 | 1 | — |
| I6 | 2-[5-(3- benzyloxycyclobutoxy)- 3-ethylsulfonyl-2- pyridyl]-3-methyl-6- (trifluoromethyl)imidazo [4,5-c]pyridine | 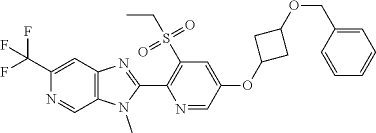 [SMILES: CCS(=O)(=O)c1cc(OC2CC(OCc3ccccc3)C2)cnc1-c1nc2cc(C(F)(F)F)ncc2n1C] | 1.13 | 547 | 1 | — |
| I7 | 3-[[5-ethylsulfonyl-6-[3- methyl-6- (trifluoromethyl)imidazo [4,5-c]pyridin-2-yl]-3- pyridyl]oxy]cyclobutanol | 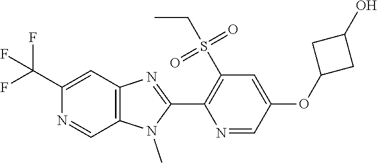 [SMILES: CCS(=O)(=O)c1cc(OC2CC(O)C2)cnc1-c1nc2cc(C(F)(F)F)ncc2n1C] | 0.86 | 457 | 1 | — |
| I8 | 3-[[5-ethylsulfonyl-6-[3- methyl-6- (trifluoromethyl)imidazo [4,5-c]pyridin-2-yl]-3- pyridyl]oxy]cyclobutanone | 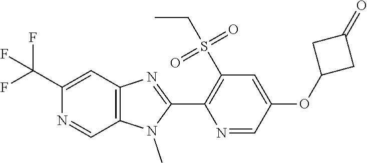 [SMILES: CCS(=O)(=O)c1cc(OC2CC(=O)C2)cnc1-c1nc2cc(C(F)(F)F)ncc2n1C] | 0.91 | 455 | 1 | — |
| I9 | 2-[3-ethylsulfonyl-4-[3- methyl-6- (trifluoromethyl)imidazo [4,5-c]pyridin-2- yl]phenoxy]-2-methyl- propanamide | 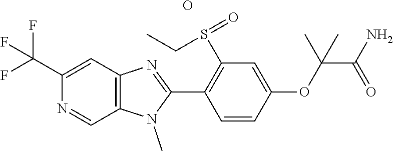 | 1.29 | 471 | 1 | — |

The following mixtures of the compounds of formula I with active ingredients are preferred (the abbreviation “TX” means “one compound selected from the group consisting of the compounds described in Tables 1-3 and P of the present invention”):

an adjuvant selected from the group of substances consisting of petroleum oils (alternative name) (628)+TX,

an acaricide selected from the group of substances consisting of 1,1-bis(4-chlorophenyl)-2-ethoxyethanol (IUPAC name) (910)+TX, 2,4-dichlorophenyl benzenesulfonate (IUPAC/Chemical Abstracts name) (1059)+TX, 2-fluoro-N-methyl-N-1-naphthylacetamide (IUPAC name) (1295)+TX, 4-chlorophenyl phenyl sulfone (IUPAC name) (981)+TX, abamectin (1)+TX, acequinocyl (3)+TX, acetoprole [CCN]+TX, acrinathrin (9)+TX, aldicarb (16)+TX, aldoxycarb (863)+TX, alpha-cypermethrin (202)+TX, amidithion (870)+TX, amidoflumet [CCN]+TX, amidothioate (872)+TX, amiton (875)+TX, amiton hydrogen oxalate (875)+TX, amitraz (24)+TX, aramite (881)+TX, arsenous oxide (882)+TX, AVI 382 (compound code)+TX, AZ 60541 (compound code)+TX, azinphos-ethyl (44)+TX, azinphos-methyl (45)+TX, azobenzene (IUPAC name) (888)+TX, azocyclotin (46)+TX, azothoate (889)+TX, benomyl (62)+TX, benoxafos (alternative name) [CCN]+TX, benzoximate (71)+TX, benzyl benzoate (IUPAC name) [CCN]+TX, bifenazate (74)+TX, bifenthrin (76)+TX, binapacryl (907)+TX, brofenvalerate (alternative name)+TX, bromocyclen (918)+TX, bromophos (920)+TX, bromophos-ethyl (921)+TX, bromopropylate (94)+TX, buprofezin (99)+TX, butocarboxim (103)+TX, butoxycarboxim (104)+TX, butylpyridaben (alternative name)+TX, calcium polysulfide (IUPAC name) (111)+TX, camphechlor (941)+TX, carbanolate (943)+TX, carbaryl (115)+TX, carbofuran (118)+TX, carbophenothion (947)+TX, CGA 50′439 (development code) (125)+TX, chinomethionat (126)+TX, chlorbenside (959)+TX, chlordimeform (964)+TX, chlordimeform hydrochloride (964)+TX, chlorfenapyr (130)+TX, chlorfenethol (968)+TX, chlorfenson (970)+TX, chlorfensulfide (971)+TX, chlorfenvinphos (131)+TX, chlorobenzilate (975)+TX, chloromebuform (977)+TX, chloromethiuron (978)+TX, chloropropylate (983)+TX, chlorpyrifos (145)+TX, chlorpyrifos-methyl (146)+TX, chlorthiophos (994)+TX, cinerin I (696)+TX, cinerin II (696)+TX, cinerins (696)+TX, clofentezine (158)+TX, closantel (alternative name) [CCN]+TX, coumaphos (174)+TX, crotamiton (alternative name) [CCN]+TX, crotoxyphos (1010)+TX, cufraneb (1013)+TX, cyanthoate (1020)+TX, cyflumetofen (CAS Reg. No.: 400882-07-7)+TX, cyhalothrin (196)+TX, cyhexatin (199)+TX, cypermethrin (201)+TX, DCPM (1032)+TX, DDT (219)+TX, demephion (1037)+TX, demephion-O (1037)+TX, demephion-S (1037)+TX, demeton (1038)+TX, demeton-methyl (224)+TX, demeton-O (1038)+TX, demeton-O-methyl (224)+TX, demeton-S (1038)+TX, demeton-S-methyl (224)+TX, demeton-S-methylsulfon (1039)+TX, diafenthiuron (226)+TX, dialifos (1042)+TX, diazinon (227)+TX, dichlofluanid (230)+TX, dichlorvos (236)+TX, dicliphos (alternative name)+TX, dicofol (242)+TX, dicrotophos (243)+TX, dienochlor (1071)+TX, dimefox (1081)+TX, dimethoate (262)+TX, dinactin (alternative name) (653)+TX, dinex (1089)+TX, dinex-diclexine (1089)+TX, dinobuton (269)+TX, dinocap (270)+TX, dinocap-4 [CCN]+TX, dinocap-6 [CCN]+TX, dinocton (1090)+TX, dinopenton (1092)+TX, dinosulfon (1097)+TX, dinoterbon (1098)+TX, dioxathion (1102)+TX, diphenyl sulfone (IUPAC name) (1103)+TX, disulfiram (alternative name) [CCN]+TX, disulfoton (278)+TX, DNOC (282)+TX, dofenapyn (1113)+TX, doramectin (alternative name) [CCN]+TX, endosulfan (294)+TX, endothion (1121)+TX, EPN (297)+TX, eprinomectin (alternative name) [CCN]+TX, ethion (309)+TX, ethoate-methyl (1134)+TX, etoxazole (320)+TX, etrimfos (1142)+TX, fenazaflor (1147)+TX, fenazaquin (328)+TX, fenbutatin oxide (330)+TX, fenothiocarb (337)+TX, fenpropathrin (342)+TX, fenpyrad (alternative name)+TX, fenpyroximate (345)+TX, fenson (1157)+TX, fentrifanil (1161)+TX, fenvalerate (349)+TX, fipronil (354)+TX, fluacrypyrim (360)+TX, fluazuron (1166)+TX, flubenzimine (1167)+TX, flucycloxuron (366)+TX, flucythrinate (367)+TX, fluenetil (1169)+TX, flufenoxuron (370)+TX, flumethrin (372)+TX, fluorbenside (1174)+TX, fluvalinate (1184)+TX, FMC 1137 (development code) (1185)+TX, formetanate (405)+TX, formetanate hydrochloride (405)+TX, formothion (1192)+TX, formparanate (1193)+TX, gamma-HCH (430)+TX, glyodin (1205)+TX, halfenprox (424)+TX, heptenophos (432)+TX, hexadecyl cyclopropanecarboxylate (IUPAC/Chemical Abstracts name) (1216)+TX, hexythiazox (441)+TX, iodomethane (IUPAC name) (542)+TX, isocarbophos (alternative name) (473)+TX, isopropyl O-(methoxyaminothiophosphoryl)salicylate (IUPAC name) (473)+TX, ivermectin (alternative name) [CCN]+TX, jasmolin I (696)+TX, jasmolin II (696)+TX, jodfenphos (1248)+TX, lindane (430)+TX, lufenuron (490)+TX, malathion (492)+TX, malonoben (1254)+TX, mecarbam (502)+TX, mephosfolan (1261)+TX, mesulfen (alternative name) [CCN]+TX, methacrifos (1266)+TX, methamidophos (527)+TX, methidathion (529)+TX, methiocarb (530)+TX, methomyl (531)+TX, methyl bromide (537)+TX, metolcarb (550)+TX, mevinphos (556)+TX, mexacarbate (1290)+TX, milbemectin (557)+TX, milbemycin oxime (alternative name) [CCN]+TX, mipafox (1293)+TX, monocrotophos (561)+TX, morphothion (1300)+TX, moxidectin (alternative name) [CCN]+TX, naled (567)+TX, NC-184 (compound code)+TX, NC-512 (compound code)+TX, nifluridide (1309)+TX, nikkomycins (alternative name) [CCN]+TX, nitrilacarb (1313)+TX, nitrilacarb 1:1 zinc chloride complex (1313)+TX, NNI-0101 (compound code)+TX, NNI-0250 (compound code)+TX, omethoate (594)+TX, oxamyl (602)+TX, oxydeprofos (1324)+TX, oxydisulfoton (1325)+TX, pp′-DDT (219)+TX, parathion (615)+TX, permethrin (626)+TX, petroleum oils (alternative name) (628)+TX, phenkapton (1330)+TX, phenthoate (631)+TX, phorate (636)+TX, phosalone (637)+TX, phosfolan (1338)+TX, phosmet (638)+TX, phosphamidon (639)+TX, phoxim (642)+TX, pirimiphos-methyl (652)+TX, polychloroterpenes (traditional name) (1347)+TX, polynactins (alternative name) (653)+TX, proclonol (1350)+TX, profenofos (662)+TX, promacyl (1354)+TX, propargite (671)+TX, propetamphos (673)+TX, propoxur (678)+TX, prothidathion (1360)+TX, prothoate (1362)+TX, pyrethrin I (696)+TX, pyrethrin II (696)+TX, pyrethrins (696)+TX, pyridaben (699)+TX, pyridaphenthion (701)+TX, pyrimidifen (706)+TX, pyrimitate (1370)+TX, quinalphos (711)+TX, quintiofos (1381)+TX, R-1492 (development code) (1382)+TX, RA-17 (development code) (1383)+TX, rotenone (722)+TX, schradan (1389)+TX, sebufos (alternative name)+TX, selamectin (alternative name) [CCN]+TX, SI-0009 (compound code)+TX, sophamide (1402)+TX, spirodiclofen (738)+TX, spiromesifen (739)+TX, SSI-121 (development code) (1404)+TX, sulfiram (alternative name) [CCN]+TX, sulfluramid (750)+TX, sulfotep (753)+TX, sulfur (754)+TX, SZI-121 (development code) (757)+TX, tau-fluvalinate (398)+TX, tebufenpyrad (763)+TX, TEPP (1417)+TX, terbam (alternative name)+TX, tetrachlorvinphos (777)+TX, tetradifon (786)+TX, tetranactin (alternative name) (653)+TX, tetrasul (1425)+TX, thiafenox (alternative name)+TX, thiocarboxime (1431)+TX, thiofanox (800)+TX, thiometon (801)+TX, thioquinox (1436)+TX, thuringiensin (alternative name) [CCN]+TX, triamiphos (1441)+TX, triarathene (1443)+TX, triazophos (820)+TX, triazuron (alternative name)+TX, trichlorfon (824)+TX, trifenofos (1455)+TX, trinactin (alternative name) (653)+TX, vamidothion (847)+TX, vaniliprole [CCN] and YI-5302 (compound code)+TX,

an algicide selected from the group of substances consisting of bethoxazin [CCN]+TX, copper dioctanoate (IUPAC name) (170)+TX, copper sulfate (172)+TX, cybutryne [CCN]+TX, dichlone (1052)+TX, dichlorophen (232)+TX, endothal (295)+TX, fentin (347)+TX, hydrated lime [CCN]+TX, nabam (566)+TX, quinoclamine (714)+TX, quinonamid (1379)+TX, simazine (730)+TX, triphenyltin acetate (IUPAC name) (347) and triphenyltin hydroxide (IUPAC name) (347)+TX,

an anthelmintic selected from the group of substances consisting of abamectin (1)+TX, crufomate (1011)+TX, doramectin (alternative name) [CCN]+TX, emamectin (291)+TX, emamectin benzoate (291)+TX, eprinomectin (alternative name) [CCN]+TX, ivermectin (alternative name) [CCN]+TX, milbemycin oxime (alternative name) [CCN]+TX, moxidectin (alternative name) [CCN]+TX, piperazine [CCN]+TX, selamectin (alternative name) [CCN]+TX, spinosad (737) and thiophanate (1435)+TX,

an avicide selected from the group of substances consisting of chloralose (127)+TX, endrin (1122)+TX, fenthion (346)+TX, pyridin-4-amine (IUPAC name) (23) and strychnine (745)+TX, a bactericide selected from the group of substances consisting of 1-hydroxy-1H-pyridine-2-thione (IUPAC name) (1222)+TX, 4-(quinoxalin-2-ylamino)benzenesulfonamide (IUPAC name) (748)+TX, 8-hydroxyquinoline sulfate (446)+TX, bronopol (97)+TX, copper dioctanoate (IUPAC name) (170)+TX, copper hydroxide (IUPAC name) (169)+TX, cresol [CCN]+TX, dichlorophen (232)+TX, dipyrithione (1105)+TX, dodicin (1112)+TX, fenaminosulf (1144)+TX, formaldehyde (404)+TX, hydrargaphen (alternative name) [CCN]+TX, kasugamycin (483)+TX, kasugamycin hydrochloride hydrate (483)+TX, nickel bis(dimethyldithiocarbamate) (IUPAC name) (1308)+TX, nitrapyrin (580)+TX, octhilinone (590)+TX, oxolinic acid (606)+TX, oxytetracycline (611)+TX, potassium hydroxyquinoline sulfate (446)+TX, probenazole (658)+TX, streptomycin (744)+TX, streptomycin sesquisulfate (744)+TX, tecloftalam (766)+TX, and thiomersal (alternative name) [CCN]+TX,

a biological agent selected from the group of substances consisting of Adoxophyes orana GV (alternative name) (12)+TX, Agrobacterium radiobacter (alternative name) (13)+TX, Amblyseius spp. (alternative name) (19)+TX, Anagrapha falcifera NPV (alternative name) (28)+TX, Anagrus atomus (alternative name) (29)+TX, Aphelinus abdominalis (alternative name) (33)+TX, Aphidius colemani (alternative name) (34)+TX, Aphidoletes aphidimyza (alternative name) (35)+TX, Autographa californica NPV (alternative name) (38)+TX, Bacillus firmus (alternative name) (48)+TX, Bacillus sphaericus Neide (scientific name) (49)+TX, Bacillus thuringiensis Berliner (scientific name) (51)+TX, Bacillus thuringiensis subsp. aizawai (scientific name) (51)+TX, Bacillus thuringiensis subsp. israelensis (scientific name) (51)+TX, Bacillus thuringiensis subsp. japonensis (scientific name) (51)+TX, Bacillus thuringiensis subsp. kurstaki (scientific name) (51)+TX, Bacillus thuringiensis subsp. tenebrionis (scientific name) (51)+TX, Beauveria bassiana (alternative name) (53)+TX, Beauveria brongniartii (alternative name) (54)+TX, Chrysoperla carnea (alternative name) (151)+TX, Cryptolaemus montrouzieri (alternative name) (178)+TX, Cydia pomonella GV (alternative name) (191)+TX, Dacnusa sibirica (alternative name) (212)+TX, Digtyphus isaea (alternative name) (254)+TX, Encarsia formosa (scientific name) (293)+TX, Eretmocerus eremicus (alternative name) (300)+TX, Helicoverpa zea NPV (alternative name) (431)+TX, Heterorhabditis bacteriophora and H. megidis (alternative name) (433)+TX, Hippodamia convergens (alternative name) (442)+TX, Leptomastix dactylopii (alternative name) (488)+TX, Macrolophus caliginosus (alternative name) (491)+TX, Mamestra brassicae NPV (alternative name) (494)+TX, Metaphycus helvolus (alternative name) (522)+TX, Metarhizium anisopliae var. acridum (scientific name) (523)+TX, Metarhizium anisopliae var. anisopliae (scientific name) (523)+TX, Neodiprion sertifer NPV and N. lecontei NPV (alternative name) (575)+TX, Orius spp. (alternative name) (596)+TX, Paecilomyces fumosoroseus (alternative name) (613)+TX, Phytoseiulus persimilis (alternative name) (644)+TX, Spodoptera exigua multicapsid nuclear polyhedrosis virus (scientific name) (741)+TX, Steinernema bibionis (alternative name) (742)+TX, Steinernema carpocapsae (alternative name) (742)+TX, Steinernema feltiae (alternative name) (742)+TX, Steinernema glaseri (alternative name) (742)+TX, Steinernema riobrave (alternative name) (742)+TX, Steinernema riobravis (alternative name) (742)+TX, Steinernema scapterisci (alternative name) (742)+TX, Steinernema spp. (alternative name) (742)+TX, Trichogramma spp. (alternative name) (826)+TX, Typhlodromus occidentalis (alternative name) (844) and Verticillium lecanii (alternative name) (848)+TX,

a soil sterilant selected from the group of substances consisting of iodomethane (IUPAC name) (542) and methyl bromide (537)+TX,

a chemosterilant selected from the group of substances consisting of apholate [CCN]+TX, bisazir (alternative name) [CCN]+TX, busulfan (alternative name) [CCN]+TX, diflubenzuron (250)+TX, dimatif (alternative name) [CCN]+TX, hemel [CCN]+TX, hempa [CCN]+TX, metepa [CCN]+TX, methiotepa [CCN]+TX, methyl apholate [CCN]+TX, morzid [CCN]+TX, penfluron (alternative name) [CCN]+TX, tepa [CCN]+TX, thiohempa (alternative name) [CCN]+TX, thiotepa (alternative name) [CCN]+TX, tretamine (alternative name) [CCN] and uredepa (alternative name) [CCN]+TX,

an insect pheromone selected from the group of substances consisting of (E)-dec-5-en-1-yl acetate with (E)-dec-5-en-1-ol (IUPAC name) (222)+TX, (E)-tridec-4-en-1-yl acetate (IUPAC name) (829)+TX, (E)-6-methylhept-2-en-4-ol (IUPAC name) (541)+TX, (E,Z)-tetradeca-4,10-dien-1-yl acetate (IUPAC name) (779)+TX, (Z)-dodec-7-en-1-yl acetate (IUPAC name) (285)+TX, (Z)-hexadec-11-enal (IUPAC name) (436)+TX, (Z)-hexadec-11-en-1-yl acetate (IUPAC name) (437)+TX, (Z)-hexadec-13-en-11-yn-1-yl acetate (IUPAC name) (438)+TX, (Z)-icos-13-en-10-one (IUPAC name) (448)+TX, (Z)-tetradec-7-en-1-al (IUPAC name) (782)+TX, (Z)-tetradec-9-en-1-ol (IUPAC name) (783)+TX, (Z)-tetradec-9-en-1-yl acetate (IUPAC name) (784)+TX, (7E,9Z)-dodeca-7,9-dien-1-yl acetate (IUPAC name) (283)+TX, (9Z,11E)-tetradeca-9,11-dien-1-yl acetate (IUPAC name) (780)+TX, (9Z,12E)-tetradeca-9,12-dien-1-yl acetate (IUPAC name) (781)+TX, 14-methyloctadec-1-ene (IUPAC name) (545)+TX, 4-methylnonan-5-ol with 4-methylnonan-5-one (IUPAC name) (544)+TX, alpha-multistriatin (alternative name) [CCN]+TX, brevicomin (alternative name) [CCN]+TX, codlelure (alternative name) [CCN]+TX, codlemone (alternative name) (167)+TX, cuelure (alternative name) (179)+TX, disparlure (277)+TX, dodec-8-en-1-yl acetate (IUPAC name) (286)+TX, dodec-9-en-1-yl acetate (IUPAC name) (287)+TX, dodeca-8+TX, 10-dien-1-yl acetate (IUPAC name) (284)+TX, dominicalure (alternative name) [CCN]+TX, ethyl 4-methyloctanoate (IUPAC name) (317)+TX, eugenol (alternative name) [CCN]+TX, frontalin (alternative name) [CCN]+TX, gossyplure (alternative name) (420)+TX, grandlure (421)+TX, grandlure I (alternative name) (421)+TX, grandlure II (alternative name) (421)+TX, grandlure III (alternative name) (421)+TX, grandlure IV (alternative name) (421)+TX, hexalure [CCN]+TX, ipsdienol (alternative name) [CCN]+TX, ipsenol (alternative name) [CCN]+TX, japonilure (alternative name) (481)+TX, lineatin (alternative name) [CCN]+TX, litlure (alternative name) [CCN]+TX, looplure (alternative name) [CCN]+TX, medlure [CCN]+TX, megatomoic acid (alternative name) [CCN]+TX, methyl eugenol (alternative name) (540)+TX, muscalure (563)+TX, octadeca-2,13-dien-1-yl acetate (IUPAC name) (588)+TX, octadeca-3,13-dien-1-yl acetate (IUPAC name) (589)+TX, orfralure (alternative name) [CCN]+TX, oryctalure (alternative name) (317)+TX, ostramone (alternative name) [CCN]+TX, siglure [CCN]+TX, sordidin (alternative name) (736)+TX, sulcatol (alternative name) [CCN]+TX, tetradec-11-en-1-yl acetate (IUPAC name) (785)+TX, trimedlure (839)+TX, trimedlure A (alternative name) (839)+TX, trimedlure B₁ (alternative name) (839)+TX, trimedlure B₂ (alternative name) (839)+TX, trimedlure C (alternative name) (839) and trunc-call (alternative name) [CCN]+TX,

an insect repellent selected from the group of substances consisting of 2-(octylthio)ethanol (IUPAC name) (591)+TX, butopyronoxyl (933)+TX, butoxy(polypropylene glycol) (936)+TX, dibutyl adipate (IUPAC name) (1046)+TX, dibutyl phthalate (1047)+TX, dibutyl succinate (IUPAC name) (1048)+TX, diethyltoluamide [CCN]+TX, dimethyl carbate [CCN]+TX, dimethyl phthalate [CCN]+TX, ethyl hexanediol (1137)+TX, hexamide [CCN]+TX, methoquin-butyl (1276)+TX, methylneodecanamide [CCN]+TX, oxamate [CCN] and picaridin [CCN]+TX, an insecticide selected from the group of substances consisting of 1-dichloro-1-nitroethane (IUPAC/Chemical Abstracts name) (1058)+TX, 1,1-dichloro-2,2-bis(4-ethylphenyl)ethane (IUPAC name) (1056), +TX, 1,2-dichloropropane (IUPAC/Chemical Abstracts name) (1062)+TX, 1,2-dichloropropane with 1,3-dichloropropene (IUPAC name) (1063)+TX, 1-bromo-2-chloroethane (IUPAC/Chemical Abstracts name) (916)+TX, 2,2,2-trichloro-1-(3,4-dichlorophenyl)ethyl acetate (IUPAC name) (1451)+TX, 2,2-dichlorovinyl 2-ethylsulfinylethyl methyl phosphate (IUPAC name) (1066)+TX, 2-(1,3-dithiolan-2-yl)phenyl dimethylcarbamate (IUPAC/Chemical Abstracts name) (1109)+TX, 2-(2-butoxyethoxy)ethyl thiocyanate (IUPAC/Chemical Abstracts name) (935)+TX, 2-(4,5-dimethyl-1,3-dioxolan-2-yl)phenyl methylcarbamate (IUPAC/Chemical Abstracts name) (1084)+TX, 2-(4-chloro-3,5-xylyloxy)ethanol (IUPAC name) (986)+TX, 2-chlorovinyl diethyl phosphate (IUPAC name) (984)+TX, 2-imidazolidone (IUPAC name) (1225)+TX, 2-isovalerylindan-1,3-dione (IUPAC name) (1246)+TX, 2-methyl(prop-2-ynyl)aminophenyl methylcarbamate (IUPAC name) (1284)+TX, 2-thiocyanatoethyl laurate (IUPAC name) (1433)+TX, 3-bromo-1-chloroprop-1-ene (IUPAC name) (917)+TX, 3-methyl-1-phenylpyrazol-5-yl dimethylcarbamate (IUPAC name) (1283)+TX, 4-methyl(prop-2-ynyl)amino-3,5-xylyl methylcarbamate (IUPAC name) (1285)+TX, 5,5-dimethyl-3-oxocyclohex-1-enyl dimethylcarbamate (IUPAC name) (1085)+TX, abamectin (1)+TX, acephate (2)+TX, acetamiprid (4)+TX, acethion (alternative name) [CCN]+TX, acetoprole [CCN]+TX, acrinathrin (9)+TX, acrylonitrile (IUPAC name) (861)+TX, alanycarb (15)+TX, aldicarb (16)+TX, aldoxycarb (863)+TX, aldrin (864)+TX, allethrin (17)+TX, allosamidin (alternative name) [CCN]+TX, allyxycarb (866)+TX, alpha-cypermethrin (202)+TX, alpha-ecdysone (alternative name) [CCN]+TX, aluminium phosphide (640)+TX, amidithion (870)+TX, amidothioate (872)+TX, aminocarb (873)+TX, amiton (875)+TX, amiton hydrogen oxalate (875)+TX, amitraz (24)+TX, anabasine (877)+TX, athidathion (883)+TX, AVI 382 (compound code)+TX, AZ 60541 (compound code)+TX, azadirachtin (alternative name) (41)+TX, azamethiphos (42)+TX, azinphos-ethyl (44)+TX, azinphos-methyl (45)+TX, azothoate (889)+TX, Bacillus thuringiensis delta endotoxins (alternative name) (52)+TX, barium hexafluorosilicate (alternative name) [CCN]+TX, barium polysulfide (IUPAC/Chemical Abstracts name) (892)+TX, barthrin [CCN]+TX, Bayer 22/190 (development code) (893)+TX, Bayer 22408 (development code) (894)+TX, bendiocarb (58)+TX, benfuracarb (60)+TX, bensultap (66)+TX, beta-cyfluthrin (194)+TX, beta-cypermethrin (203)+TX, bifenthrin (76)+TX, bioallethrin (78)+TX, bioallethrin S-cyclopentenyl isomer (alternative name) (79)+TX, bioethanomethrin [CCN]+TX, biopermethrin (908)+TX, bioresmethrin (80)+TX, bis(2-chloroethyl) ether (IUPAC name) (909)+TX, bistrifluron (83)+TX, borax (86)+TX, brofenvalerate (alternative name)+TX, bromfenvinfos (914)+TX, bromocyclen (918)+TX, bromo-DDT (alternative name) [CCN]+TX, bromophos (920)+TX, bromophos-ethyl (921)+TX, bufencarb (924)+TX, buprofezin (99)+TX, butacarb (926)+TX, butathiofos (927)+TX, butocarboxim (103)+TX, butonate (932)+TX, butoxycarboxim (104)+TX, butylpyridaben (alternative name)+TX, cadusafos (109)+TX, calcium arsenate [CCN]+TX, calcium cyanide (444)+TX, calcium polysulfide (IUPAC name) (111)+TX, camphechlor (941)+TX, carbanolate (943)+TX, carbaryl (115)+TX, carbofuran (118)+TX, carbon disulfide (IUPAC/Chemical Abstracts name) (945)+TX, carbon tetrachloride (IUPAC name) (946)+TX, carbophenothion (947)+TX, carbosulfan (119)+TX, cartap (123)+TX, cartap hydrochloride (123)+TX, cevadine (alternative name) (725)+TX, chlorbicyclen (960)+TX, chlordane (128)+TX, chlordecone (963)+TX, chlordimeform (964)+TX, chlordimeform hydrochloride (964)+TX, chlorethoxyfos (129)+TX, chlorfenapyr (130)+TX, chlorfenvinphos (131)+TX, chlorfluazuron (132)+TX, chlormephos (136)+TX, chloroform [CCN]+TX, chloropicrin (141)+TX, chlorphoxim (989)+TX, chlorprazophos (990)+TX, chlorpyrifos (145)+TX, chlorpyrifos-methyl (146)+TX, chlorthiophos (994)+TX, chromafenozide (150)+TX, cinerin I (696)+TX, cinerin II (696)+TX, cinerins (696)+TX, cis-resmethrin (alternative name)+TX, cismethrin (80)+TX, clocythrin (alternative name)+TX, cloethocarb (999)+TX, closantel (alternative name) [CCN]+TX, clothianidin (165)+TX, copper acetoarsenite [CCN]+TX, copper arsenate [CCN]+TX, copper oleate [CCN]+TX, coumaphos (174)+TX, coumithoate (1006)+TX, crotamiton (alternative name) [CCN]+TX, crotoxyphos (1010)+TX, crufomate (1011)+TX, cryolite (alternative name) (177)+TX, CS 708 (development code) (1012)+TX, cyanofenphos (1019)+TX, cyanophos (184)+TX, cyanthoate (1020)+TX, cyclethrin [CCN]+TX, cycloprothrin (188)+TX, cyfluthrin (193)+TX, cyhalothrin (196)+TX, cypermethrin (201)+TX, cyphenothrin (206)+TX, cyromazine (209)+TX, cythioate (alternative name) [CCN]+TX, d-limonene (alternative name) [CCN]+TX, d-tetramethrin (alternative name) (788)+TX, DAEP (1031)+TX, dazomet (216)+TX, DDT (219)+TX, decarbofuran (1034)+TX, deltamethrin (223)+TX, demephion (1037)+TX, demephion-O (1037)+TX, demephion-S (1037)+TX, demeton (1038)+TX, demeton-methyl (224)+TX, demeton-O (1038)+TX, demeton-O-methyl (224)+TX, demeton-S (1038)+TX, demeton-S-methyl (224)+TX, demeton-S-methylsulphon (1039)+TX, diafenthiuron (226)+TX, dialifos (1042)+TX, diamidafos (1044)+TX, diazinon (227)+TX, dicapthon (1050)+TX, dichlofenthion (1051)+TX, dichlorvos (236)+TX, dicliphos (alternative name)+TX, dicresyl (alternative name) [CCN]+TX, dicrotophos (243)+TX, dicyclanil (244)+TX, dieldrin (1070)+TX, diethyl 5-methylpyrazol-3-yl phosphate (IUPAC name) (1076)+TX, diflubenzuron (250)+TX, dilor (alternative name) [CCN]+TX, dimefluthrin [CCN]+TX, dimefox (1081)+TX, dimetan (1085)+TX, dimethoate (262)+TX, dimethrin (1083)+TX, dimethylvinphos (265)+TX, dimetilan (1086)+TX, dinex (1089)+TX, dinex-diclexine (1089)+TX, dinoprop (1093)+TX, dinosam (1094)+TX, dinoseb (1095)+TX, dinotefuran (271)+TX, diofenolan (1099)+TX, dioxabenzofos (1100)+TX, dioxacarb (1101)+TX, dioxathion (1102)+TX, disulfoton (278)+TX, dithicrofos (1108)+TX, DNOC (282)+TX, doramectin (alternative name) [CCN]+TX, DSP (1115)+TX, ecdysterone (alternative name) [CCN]+TX, EI 1642 (development code) (1118)+TX, emamectin (291)+TX, emamectin benzoate (291)+TX, EMPC (1120)+TX, empenthrin (292)+TX, endosulfan (294)+TX, endothion (1121)+TX, endrin (1122)+TX, EPBP (1123)+TX, EPN (297)+TX, epofenonane (1124)+TX, eprinomectin (alternative name) [CCN]+TX, esfenvalerate (302)+TX, etaphos (alternative name) [CCN]+TX, ethiofencarb (308)+TX, ethion (309)+TX, ethiprole (310)+TX, ethoate-methyl (1134)+TX, ethoprophos (312)+TX, ethyl formate (IUPAC name) [CCN]+TX, ethyl-DDD (alternative name) (1056)+TX, ethylene dibromide (316)+TX, ethylene dichloride (chemical name) (1136)+TX, ethylene oxide [CCN]+TX, etofenprox (319)+TX, etrimfos (1142)+TX, EXD (1143)+TX, famphur (323)+TX, fenamiphos (326)+TX, fenazaflor (1147)+TX, fenchlorphos (1148)+TX, fenethacarb (1149)+TX, fenfluthrin (1150)+TX, fenitrothion (335)+TX, fenobucarb (336)+TX, fenoxacrim (1153)+TX, fenoxycarb (340)+TX, fenpirithrin (1155)+TX, fenpropathrin (342)+TX, fenpyrad (alternative name)+TX, fensulfothion (1158)+TX, fenthion (346)+TX, fenthion-ethyl [CCN]+TX, fenvalerate (349)+TX, fipronil (354)+TX, flonicamid (358)+TX, flubendiamide (CAS. Reg. No.: 272451-65-7)+TX, flucofuron (1168)+TX, flucycloxuron (366)+TX, flucythrinate (367)+TX, fluenetil (1169)+TX, flufenerim [CCN]+TX, flufenoxuron (370)+TX, flufenprox (1171)+TX, flumethrin (372)+TX, fluvalinate (1184)+TX, FMC 1137 (development code) (1185)+TX, fonofos (1191)+TX, formetanate (405)+TX, formetanate hydrochloride (405)+TX, formothion (1192)+TX, formparanate (1193)+TX, fosmethilan (1194)+TX, fospirate (1195)+TX, fosthiazate (408)+TX, fosthietan (1196)+TX, furathiocarb (412)+TX, furethrin (1200)+TX, gamma-cyhalothrin (197)+TX, gamma-HCH (430)+TX, guazatine (422)+TX, guazatine acetates (422)+TX, GY-81 (development code) (423)+TX, halfenprox (424)+TX, halofenozide (425)+TX, HCH (430)+TX, HEOD (1070)+TX, heptachlor (1211)+TX, heptenophos (432)+TX, heterophos [CCN]+TX, hexaflumuron (439)+TX, HHDN (864)+TX, hydramethylnon (443)+TX, hydrogen cyanide (444)+TX, hydroprene (445)+TX, hyquincarb (1223)+TX, imidacloprid (458)+TX, imiprothrin (460)+TX, indoxacarb (465)+TX, iodomethane (IUPAC name) (542)+TX, IPSP (1229)+TX, isazofos (1231)+TX, isobenzan (1232)+TX, isocarbophos (alternative name) (473)+TX, isodrin (1235)+TX, isofenphos (1236)+TX, isolane (1237)+TX, isoprocarb (472)+TX, isopropyl O-(methoxy-aminothiophosphoryl)salicylate (IUPAC name) (473)+TX, isoprothiolane (474)+TX, isothioate (1244)+TX, isoxathion (480)+TX, ivermectin (alternative name) [CCN]+TX, jasmolin I (696)+TX, jasmolin II (696)+TX, jodfenphos (1248)+TX, juvenile hormone I (alternative name) [CCN]+TX, juvenile hormone II (alternative name) [CCN]+TX, juvenile hormone III (alternative name) [CCN]+TX, kelevan (1249)+TX, kinoprene (484)+TX, lambda-cyhalothrin (198)+TX, lead arsenate [CCN]+TX, lepimectin (CCN)+TX, leptophos (1250)+TX, lindane (430)+TX, lirimfos (1251)+TX, lufenuron (490)+TX, lythidathion (1253)+TX, m-cumenyl methylcarbamate (IUPAC name) (1014)+TX, magnesium phosphide (IUPAC name) (640)+TX, malathion (492)+TX, malonoben (1254)+TX, mazidox (1255)+TX, mecarbam (502)+TX, mecarphon (1258)+TX, menazon (1260)+TX, mephosfolan (1261)+TX, mercurous chloride (513)+TX, mesulfenfos (1263)+TX, metaflumizone (CCN)+TX, metam (519)+TX, metam-potassium (alternative name) (519)+TX, metam-sodium (519)+TX, methacrifos (1266)+TX, methamidophos (527)+TX, methanesulfonyl fluoride (IUPAC/Chemical Abstracts name) (1268)+TX, methidathion (529)+TX, methiocarb (530)+TX, methocrotophos (1273)+TX, methomyl (531)+TX, methoprene (532)+TX, methoquin-butyl (1276)+TX, methothrin (alternative name) (533)+TX, methoxychlor (534)+TX, methoxyfenozide (535)+TX, methyl bromide (537)+TX, methyl isothiocyanate (543)+TX, methylchloroform (alternative name) [CCN]+TX, methylene chloride [CCN]+TX, metofluthrin [CCN]+TX, metolcarb (550)+TX, metoxadiazone (1288)+TX, mevinphos (556)+TX, mexacarbate (1290)+TX, milbemectin (557)+TX, milbemycin oxime (alternative name) [CCN]+TX, mipafox (1293)+TX, mirex (1294)+TX, monocrotophos (561)+TX, morphothion (1300)+TX, moxidectin (alternative name) [CCN]+TX, naftalofos (alternative name) [CCN]+TX, naled (567)+TX, naphthalene (IUPAC/Chemical Abstracts name) (1303)+TX, NC-170 (development code) (1306)+TX, NC-184 (compound code)+TX, nicotine (578)+TX, nicotine sulfate (578)+TX, nifluridide (1309)+TX, nitenpyram (579)+TX, nithiazine (1311)+TX, nitrilacarb (1313)+TX, nitrilacarb 1:1 zinc chloride complex (1313)+TX, NNI-0101 (compound code)+TX, NNI-0250 (compound code)+TX, nornicotine (traditional name) (1319)+TX, novaluron (585)+TX, noviflumuron (586)+TX, O-5-dichloro-4-iodophenyl O-ethyl ethylphosphonothioate (IUPAC name) (1057)+TX, 0,0-diethyl O-4-methyl-2-oxo-2H-chromen-7-yl phosphorothioate (IUPAC name) (1074)+TX, 0,0-diethyl O-6-methyl-2-propylpyrimidin-4-yl phosphorothioate (IUPAC name) (1075)+TX, O,O,O′,O′-tetrapropyl dithiopyrophosphate (IUPAC name) (1424)+TX, oleic acid (IUPAC name) (593)+TX, omethoate (594)+TX, oxamyl (602)+TX, oxydemeton-methyl (609)+TX, oxydeprofos (1324)+TX, oxydisulfoton (1325)+TX, pp′-DDT (219)+TX, para-dichlorobenzene [CCN]+TX, parathion (615)+TX, parathion-methyl (616)+TX, penfluron (alternative name) [CCN]+TX, pentachlorophenol (623)+TX, pentachlorophenyl laurate (IUPAC name) (623)+TX, permethrin (626)+TX, petroleum oils (alternative name) (628)+TX, PH 60-38 (development code) (1328)+TX, phenkapton (1330)+TX, phenothrin (630)+TX, phenthoate (631)+TX, phorate (636)+TX, phosalone (637)+TX, phosfolan (1338)+TX, phosmet (638)+TX, phosnichlor (1339)+TX, phosphamidon (639)+TX, phosphine (IUPAC name) (640)+TX, phoxim (642)+TX, phoxim-methyl (1340)+TX, pirimetaphos (1344)+TX, pirimicarb (651)+TX, pirimiphos-ethyl (1345)+TX, pirimiphos-methyl (652)+TX, polychlorodicyclopentadiene isomers (IUPAC name) (1346)+TX, polychloroterpenes (traditional name) (1347)+TX, potassium arsenite [CCN]+TX, potassium thiocyanate [CCN]+TX, prallethrin (655)+TX, precocene I (alternative name) [CCN]+TX, precocene II (alternative name) [CCN]+TX, precocene III (alternative name) [CCN]+TX, primidophos (1349)+TX, profenofos (662)+TX, profluthrin [CCN]+TX, promacyl (1354)+TX, promecarb (1355)+TX, propaphos (1356)+TX, propetamphos (673)+TX, propoxur (678)+TX, prothidathion (1360)+TX, prothiofos (686)+TX, prothoate (1362)+TX, protrifenbute [CCN]+TX, pymetrozine (688)+TX, pyraclofos (689)+TX, pyrazophos (693)+TX, pyresmethrin (1367)+TX, pyrethrin I (696)+TX, pyrethrin II (696)+TX, pyrethrins (696)+TX, pyridaben (699)+TX, pyridalyl (700)+TX, pyridaphenthion (701)+TX, pyrimidifen (706)+TX, pyrimitate (1370)+TX, pyriproxyfen (708)+TX, quassia (alternative name) [CCN]+TX, quinalphos (711)+TX, quinalphos-methyl (1376)+TX, quinothion (1380)+TX, quintiofos (1381)+TX, R-1492 (development code) (1382)+TX, rafoxanide (alternative name) [CCN]+TX, resmethrin (719)+TX, rotenone (722)+TX, RU 15525 (development code) (723)+TX, RU 25475 (development code) (1386)+TX, ryania (alternative name) (1387)+TX, ryanodine (traditional name) (1387)+TX, sabadilla (alternative name) (725)+TX, schradan (1389)+TX, sebufos (alternative name)+TX, selamectin (alternative name) [CCN]+TX, SI-0009 (compound code)+TX, SI-0205 (compound code)+TX, SI-0404 (compound code)+TX, SI-0405 (compound code)+TX, silafluofen (728)+TX, SN 72129 (development code) (1397)+TX, sodium arsenite [CCN]+TX, sodium cyanide (444)+TX, sodium fluoride (IUPAC/Chemical Abstracts name) (1399)+TX, sodium hexafluorosilicate (1400)+TX, sodium pentachlorophenoxide (623)+TX, sodium selenate (IUPAC name) (1401)+TX, sodium thiocyanate [CCN]+TX, sophamide (1402)+TX, spinosad (737)+TX, spiromesifen (739)+TX, spirotetrmat (CCN)+TX, sulcofuron (746)+TX, sulcofuron-sodium (746)+TX, sulfluramid (750)+TX, sulfotep (753)+TX, sulfuryl fluoride (756)+TX, sulprofos (1408)+TX, tar oils (alternative name) (758)+TX, tau-fluvalinate (398)+TX, tazimcarb (1412)+TX, TDE (1414)+TX, tebufenozide (762)+TX, tebufenpyrad (763)+TX, tebupirimfos (764)+TX, teflubenzuron (768)+TX, tefluthrin (769)+TX, temephos (770)+TX, TEPP (1417)+TX, terallethrin (1418)+TX, terbam (alternative name)+TX, terbufos (773)+TX, tetrachloroethane [CCN]+TX, tetrachlorvinphos (777)+TX, tetramethrin (787)+TX, theta-cypermethrin (204)+TX, thiacloprid (791)+TX, thiafenox (alternative name)+TX, thiamethoxam (792)+TX, thicrofos (1428)+TX, thiocarboxime (1431)+TX, thiocyclam (798)+TX, thiocyclam hydrogen oxalate (798)+TX, thiodicarb (799)+TX, thiofanox (800)+TX, thiometon (801)+TX, thionazin (1434)+TX, thiosultap (803)+TX, thiosultap-sodium (803)+TX, thuringiensin (alternative name) [CCN]+TX, tolfenpyrad (809)+TX, tralomethrin (812)+TX, transfluthrin (813)+TX, transpermethrin (1440)+TX, triamiphos (1441)+TX, triazamate (818)+TX, triazophos (820)+TX, triazuron (alternative name)+TX, trichlorfon (824)+TX, trichlormetaphos-3 (alternative name) [CCN]+TX, trichloronat (1452)+TX, trifenofos (1455)+TX, triflumuron (835)+TX, trimethacarb (840)+TX, triprene (1459)+TX, vamidothion (847)+TX, vaniliprole [CCN]+TX, veratridine (alternative name) (725)+TX, veratrine (alternative name) (725)+TX, XMC (853)+TX, xylylcarb (854)+TX, YI-5302 (compound code)+TX, zeta-cypermethrin (205)+TX, zetamethrin (alternative name)+TX, zinc phosphide (640)+TX, zolaprofos (1469) and ZXI 8901 (development code) (858)+TX, cyantraniliprole [736994-63-19+TX, chlorantraniliprole [500008-45-7]+TX, cyenopyrafen [560121-52-0]+TX, cyflumetofen [400882-07-7]+TX, pyrifluquinazon [337458-27-2]+TX, spinetoram [187166-40-1+187166-15-0]+TX, spirotetramat [203313-25-1]+TX, sulfoxaflor [946578-00-3]+TX, flufiprole [704886-18-0]+TX, meperfluthrin [915288-13-0]+TX, tetramethylfluthrin [84937-88-2]+TX, triflumezopyrim (disclosed in WO 2012/092115)+TX, fluxametamide (WO 2007/026965)+TX, epsilon-metofluthrin [240494-71-7]+TX, epsilon-momfluorothrin [1065124-65-3]+TX, fluazaindolizine [1254304-22-7]+TX, chloroprallethrin [399572-87-3]+TX, fluxametamide [928783-29-3]+TX, cyhalodiamide [1262605-53-7]+TX, tioxazafen [330459-31-9]+TX, broflanilide [1207727-04-5]+TX, flufiprole [704886-18-0]+TX, cyclaniliprole [1031756-98-5]+TX, tetraniliprole [1229654-66-3]+TX, guadipyr (described in WO2010/060231)+TX, cycloxaprid (described in WO 2005/077934)+TX, spiropidion+TX, Afidopyropen+TX, flupyrimin+TX, Momfluorothrin+TX, kappa-bifenthrin+TX, kappa-tefluthrin+TX, Dichloromezotiaz+TX, Tetrachloraniliprole+TX, benzpyrimoxan+TX;

a molluscicide selected from the group of substances consisting of bis(tributyltin) oxide (IUPAC name) (913)+TX, bromoacetamide [CCN]+TX, calcium arsenate [CCN]+TX, cloethocarb (999)+TX, copper acetoarsenite [CCN]+TX, copper sulfate (172)+TX, fentin (347)+TX, ferric phosphate (IUPAC name) (352)+TX, metaldehyde (518)+TX, methiocarb (530)+TX, niclosamide (576)+TX, niclosamide-olamine (576)+TX, pentachlorophenol (623)+TX, sodium pentachlorophenoxide (623)+TX, tazimcarb (1412)+TX, thiodicarb (799)+TX, tributyltin oxide (913)+TX, trifenmorph (1454)+TX, trimethacarb (840)+TX, triphenyltin acetate (IUPAC name) (347) and triphenyltin hydroxide (IUPAC name) (347)+TX, pyriprole [394730-71-3]+TX,

a nematicide selected from the group of substances consisting of AKD-3088 (compound code)+TX, 1,2-dibromo-3-chloropropane (IUPAC/Chemical Abstracts name) (1045)+TX, 1,2-dichloropropane (IUPAC/Chemical Abstracts name) (1062)+TX, 1,2-dichloropropane with 1,3-dichloropropene (IUPAC name) (1063)+TX, 1,3-dichloropropene (233)+TX, 3,4-dichlorotetrahydrothiophene 1,1-dioxide (IUPAC/Chemical Abstracts name) (1065)+TX, 3-(4-chlorophenyl)-5-methylrhodanine (IUPAC name) (980)+TX, 5-methyl-6-thioxo-1,3,5-thiadiazinan-3-ylacetic acid (IUPAC name) (1286)+TX, 6-isopentenylaminopurine (alternative name) (210)+TX, abamectin (1)+TX, acetoprole [CCN]+TX, alanycarb (15)+TX, aldicarb (16)+TX, aldoxycarb (863)+TX, AZ 60541 (compound code)+TX, benclothiaz [CCN]+TX, benomyl (62)+TX, butylpyridaben (alternative name)+TX, cadusafos (109)+TX, carbofuran (118)+TX, carbon disulfide (945)+TX, carbosulfan (119)+TX, chloropicrin (141)+TX, chlorpyrifos (145)+TX, cloethocarb (999)+TX, cytokinins (alternative name) (210)+TX, dazomet (216)+TX, DBCP (1045)+TX, DCIP (218)+TX, diamidafos (1044)+TX, dichlofenthion (1051)+TX, dicliphos (alternative name)+TX, dimethoate (262)+TX, doramectin (alternative name) [CCN]+TX, emamectin (291)+TX, emamectin benzoate (291)+TX, eprinomectin (alternative name) [CCN]+TX, ethoprophos (312)+TX, ethylene dibromide (316)+TX, fenamiphos (326)+TX, fenpyrad (alternative name)+TX, fensulfothion (1158)+TX, fosthiazate (408)+TX, fosthietan (1196)+TX, furfural (alternative name) [CCN]+TX, GY-81 (development code) (423)+TX, heterophos [CCN]+TX, iodomethane (IUPAC name) (542)+TX, isamidofos (1230)+TX, isazofos (1231)+TX, ivermectin (alternative name) [CCN]+TX, kinetin (alternative name) (210)+TX, mecarphon (1258)+TX, metam (519)+TX, metam-potassium (alternative name) (519)+TX, metam-sodium (519)+TX, methyl bromide (537)+TX, methyl isothiocyanate (543)+TX, milbemycin oxime (alternative name) [CCN]+TX, moxidectin (alternative name) [CCN]+TX, Myrothecium verrucaria composition (alternative name) (565)+TX, NC-184 (compound code)+TX, oxamyl (602)+TX, phorate (636)+TX, phosphamidon (639)+TX, phosphocarb [CCN]+TX, sebufos (alternative name)+TX, selamectin (alternative name) [CCN]+TX, spinosad (737)+TX, terbam (alternative name)+TX, terbufos (773)+TX, tetrachlorothiophene (IUPAC/Chemical Abstracts name) (1422)+TX, thiafenox (alternative name)+TX, thionazin (1434)+TX, triazophos (820)+TX, triazuron (alternative name)+TX, xylenols [CCN]+TX, YI-5302 (compound code) and zeatin (alternative name) (210)+TX, fluensulfone [318290-98-1]+TX, fluopyram+TX,

a nitrification inhibitor selected from the group of substances consisting of potassium ethylxanthate [CCN] and nitrapyrin (580)+TX,

a plant activator selected from the group of substances consisting of acibenzolar (6)+TX, acibenzolar-S-methyl (6)+TX, probenazole (658) and Reynoutria sachalinensis extract (alternative name) (720)+TX,

a rodenticide selected from the group of substances consisting of 2-isovalerylindan-1,3-dione (IUPAC name) (1246)+TX, 4-(quinoxalin-2-ylamino)benzenesulfonamide (IUPAC name) (748)+TX, alpha-chlorohydrin [CCN]+TX, aluminium phosphide (640)+TX, antu (880)+TX, arsenous oxide (882)+TX, barium carbonate (891)+TX, bisthiosemi (912)+TX, brodifacoum (89)+TX, bromadiolone (91)+TX, bromethalin (92)+TX, calcium cyanide (444)+TX, chloralose (127)+TX, chlorophacinone (140)+TX, cholecalciferol (alternative name) (850)+TX, coumachlor (1004)+TX, coumafuryl (1005)+TX, coumatetralyl (175)+TX, crimidine (1009)+TX, difenacoum (246)+TX, difethialone (249)+TX, diphacinone (273)+TX, ergocalciferol (301)+TX, flocoumafen (357)+TX, fluoroacetamide (379)+TX, flupropadine (1183)+TX, flupropadine hydrochloride (1183)+TX, gamma-HCH (430)+TX, HCH (430)+TX, hydrogen cyanide (444)+TX, iodomethane (IUPAC name) (542)+TX, lindane (430)+TX, magnesium phosphide (IUPAC name) (640)+TX, methyl bromide (537)+TX, norbormide (1318)+TX, phosacetim (1336)+TX, phosphine (IUPAC name) (640)+TX, phosphorus [CCN]+TX, pindone (1341)+TX, potassium arsenite [CCN]+TX, pyrinuron (1371)+TX, scilliroside (1390)+TX, sodium arsenite [CCN]+TX, sodium cyanide (444)+TX, sodium fluoroacetate (735)+TX, strychnine (745)+TX, thallium sulfate [CCN]+TX, warfarin (851) and zinc phosphide (640)+TX,

a synergist selected from the group of substances consisting of 2-(2-butoxyethoxy)ethyl piperonylate (IUPAC name) (934)+TX, 5-(1,3-benzodioxol-5-yl)-3-hexylcyclohex-2-enone (IUPAC name) (903)+TX, farnesol with nerolidol (alternative name) (324)+TX, MB-599 (development code) (498)+TX, MGK 264 (development code) (296)+TX, piperonyl butoxide (649)+TX, piprotal (1343)+TX, propyl isomer (1358)+TX, S421 (development code) (724)+TX, sesamex (1393)+TX, sesasmolin (1394) and sulfoxide (1406)+TX,

an animal repellent selected from the group of substances consisting of anthraquinone (32)+TX, chloralose (127)+TX, copper naphthenate [CCN]+TX, copper oxychloride (171)+TX, diazinon (227)+TX, dicyclopentadiene (chemical name) (1069)+TX, guazatine (422)+TX, guazatine acetates (422)+TX, methiocarb (530)+TX, pyridin-4-amine (IUPAC name) (23)+TX, thiram (804)+TX, trimethacarb (840)+TX, zinc naphthenate [CCN] and ziram (856)+TX, a virucide selected from the group of substances consisting of imanin (alternative name) [CCN] and ribavirin (alternative name) [CCN]+TX,

a wound protectant selected from the group of substances consisting of mercuric oxide (512)+TX, octhilinone (590) and thiophanate-methyl (802)+TX,

and biologically active compounds selected from the group consisting of azaconazole (60207-31-0]+TX, bitertanol [70585-36-3]+TX, bromuconazole [116255-48-2]+TX, cyproconazole [94361-06-5]+TX, difenoconazole [119446-68-3]+TX, diniconazole [83657-24-3]+TX, epoxiconazole [106325-08-0]+TX, fenbuconazole [114369-43-6]+TX, fluquinconazole [136426-54-5]+TX, flusilazole [85509-19-9]+TX, flutriafol [76674-21-0]+TX, hexaconazole [79983-71-4]+TX, imazalil [35554-44-0]+TX, imibenconazole [86598-92-7]+TX, ipconazole [125225-28-7]+TX, metconazole [125116-23-6]+TX, myclobutanil [88671-89-0]+TX, pefurazoate [101903-30-4]+TX, penconazole [66246-88-6]+TX, prothioconazole [178928-70-6]+TX, pyrifenox [88283-41-4]+TX, prochloraz [67747-09-5]+TX, propiconazole [60207-90-1]+TX, simeconazole [149508-90-7]+TX, tebuconazole [107534-96-3]+TX, tetraconazole [112281-77-3]+TX, triadimefon [43121-43-3]+TX, triadimenol [55219-65-3]+TX, triflumizole [99387-89-0]+TX, triticonazole [131983-72-7]+TX, ancymidol [12771-68-5]+TX, fenarimol [60168-88-9]+TX, nuarimol [63284-71-9]+TX, bupirimate [41483-43-6]+TX, dimethirimol [5221-53-4]+TX, ethirimol [23947-60-6]+TX, dodemorph [1593-77-7]+TX, fenpropidine [67306-00-7]+TX, fenpropimorph [67564-91-4]+TX, spiroxamine [118134-30-8]+TX, tridemorph [81412-43-3]+TX, cyprodinil [121552-61-2]+TX, mepanipyrim [110235-47-7]+TX, pyrimethanil [53112-28-0]+TX, fenpiclonil [74738-17-3]+TX, fludioxonil [131341-86-1]+TX, benalaxyl [71626-11-4]+TX, furalaxyl [57646-30-7]+TX, metalaxyl [57837-19-1]+TX, R-metalaxyl [70630-17-0]+TX, ofurace [58810-48-3]+TX, oxadixyl [77732-09-3]+TX, benomyl [17804-35-2]+TX, carbendazim [10605-21-7]+TX, debacarb [62732-91-6]+TX, fuberidazole [3878-19-1]+TX, thiabendazole [148-79-8]+TX, chlozolinate [84332-86-5]+TX, dichlozoline [24201-58-9]+TX, iprodione [36734-19-7]+TX, myclozoline [54864-61-8]+TX, procymidone [32809-16-8]+TX, vinclozoline [50471-44-8]+TX, boscalid [188425-85-6]+TX, carboxin [5234-68-4]+TX, fenfuram [24691-80-3]+TX, flutolanil [66332-96-5]+TX, mepronil [55814-41-0]+TX, oxycarboxin [5259-88-1]+TX, penthiopyrad [183675-82-3]+TX, thifluzamide [130000-40-7]+TX, guazatine [108173-90-6]+TX, dodine [2439-10-3] [112-65-2] (free base)+TX, iminoctadine [13516-27-3]+TX, azoxystrobin [131860-33-8]+TX, dimoxystrobin [149961-52-4]+TX, enestroburin {Proc. BCPC, Int. Congr., Glasgow, 2003, 1, 93}+TX, fluoxastrobin [361377-29-9]+TX, kresoxim-methyl [143390-89-0]+TX, metominostrobin [133408-50-1]+TX, trifloxystrobin [141517-21-7]+TX, orysastrobin [248593-16-0]+TX, picoxystrobin [117428-22-5]+TX, pyraclostrobin [175013-18-0]+TX, ferbam [14484-64-1]+TX, mancozeb [8018-01-7]+TX, maneb [12427-38-2]+TX, metiram [9006-42-2]+TX, propineb [12071-83-9]+TX, thiram [137-26-8]+TX, zineb [12122-67-7]+TX, ziram [137-30-4]+TX, captafol [2425-06-1]+TX, captan [133-06-2]+TX, dichlofluanid [1085-98-9]+TX, fluoroimide [41205-21-4]+TX, folpet [133-07-3]+TX, tolylfluanid [731-27-1]+TX, bordeaux mixture [8011-63-0]+TX, copperhydroxid [20427-59-2]+TX, copperoxychlorid [1332-40-7]+TX, coppersulfat [7758-98-7]+TX, copperoxid [1317-39-1]+TX, mancopper [53988-93-5]+TX, oxine-copper [10380-28-6]+TX, dinocap [131-72-6]+TX, nitrothal-isopropyl [10552-74-6]+TX, edifenphos [17109-49-8]+TX, iprobenphos [26087-47-8]+TX, isoprothiolane [50512-35-1]+TX, phosdiphen [36519-00-3]+TX, pyrazophos [13457-18-6]+TX, tolclofos-methyl [57018-04-9]+TX, acibenzo-lar-S-methyl [135158-54-2]+TX, anilazine [101-05-3]+TX, benthiavalicarb [413615-35-7]+TX, blasticidin-S [2079-00-7]+TX, chinomethionat [2439-01-2]+TX, chloroneb [2675-77-6]+TX, chlorothalonil [1897-45-6]+TX, cyflufenamid [180409-60-3]+TX, cymoxanil [57966-95-7]+TX, dichlone [117-80-6]+TX, diclocymet [139920-32-4]+TX, diclomezine [62865-36-5]+TX, dicloran [99-30-9]+TX, diethofencarb [87130-20-9]+TX, dimethomorph [110488-70-5]+TX, SYP-L190 (Flumorph) [211867-47-9]+TX, dithianon [3347-22-6]+TX, ethaboxam [162650-77-3]+TX, etridiazole [2593-15-9]+TX, famoxadone [131807-57-3]+TX, fenamidone [161326-34-7]+TX, fenoxanil [115852-48-7]+TX, fentin [668-34-8]+TX, ferimzone [89269-64-7]+TX, fluazinam [79622-59-6]+TX, fluopicolide [239110-15-7]+TX, flusulfamide [106917-52-6]+TX, fenhexamid [126833-17-8]+TX, fosetyl-aluminium [39148-24-8]+TX, hymexazol [10004-44-1]+TX, iprovalicarb [140923-17-7]+TX, IKF-916 (Cyazofamid) [120116-88-3]+TX, kasugamycin [6980-18-3]+TX, methasulfocarb [66952-49-6]+TX, metrafenone [220899-03-6]+TX, pencycuron [66063-05-6]+TX, phthalide [27355-22-2]+TX, polyoxins [11113-80-7]+TX, probenazole [27605-76-1]+TX, propamocarb [25606-41-1]+TX, proquinazid [189278-12-4]+TX, pyroquilon [57369-32-1]+TX, quinoxyfen [124495-18-7]+TX, quintozene [82-68-8]+TX, sulfur [7704-34-9]+TX, tiadinil [223580-51-6]+TX, triazoxide [72459-58-6]+TX, tricyclazole [41814-78-2]+TX, triforine [26644-46-2]+TX, validamycin [37248-47-8]+TX, zoxamide (RH7281) [156052-68-5]+TX, mandipropamid [374726-62-2]+TX, isopyrazam [881685-58-1]+TX, sedaxane [874967-67-6]+TX, 3-difluoromethyl-1-methyl-1H-pyrazole-4-carboxylic acid (9-dichloromethylene-1,2,3,4-tetrahydro-1,4-methano-naphthalen-5-yl)-amide (disclosed in WO 2007/048556)+TX, 3-difluoromethyl-1-methyl-1H-pyrazole-4-carboxylic acid (3′,4′,5′-trifluoro-biphenyl-2-yl)-amide (disclosed in WO 2006/087343)+TX, [(3S,4R,4aR,6S,6aS,12R,12aS,12bS)-3-[(cyclopropylcarbonyl)oxy]-1,3,4,4a,5,6,6a,12,12a,12b-decahydro-6,12-dihydroxy-4,6a,12b-trimethyl-11-oxo-9-(3-pyridinyl)-2H,11Hnaphtho[2,1-b]pyrano[3,4-e]pyran-4-yl]methyl-cyclopropanecarboxylate [915972-17-7]+TX and 1,3,5-trimethyl-N-(2-methyl-1-oxopropyl)-N-[3-(2-methylpropyl)-4-[2,2,2-trifluoro-1-methoxy-1-(trifluoromethyl)ethyl]phenyl]-1H-pyrazole-4-carboxamide [926914-55-8]+TX, lancotrione [1486617-21-3]+TX, florpyrauxifen [943832-81-3]+TX, ipfentrifluconazole[1417782-08-1]+TX, mefentrifluconazole [1417782-03-6]+TX, quinofumelin [861647-84-9]+TX, chloroprallethrin [399572-87-3]+TX, cyhalodiamide [1262605-53-7]+TX, fluazaindolizine [1254304-22-7]+TX, fluxametamide [928783-29-3]+TX, epsilon-metofluthrin [240494-71-7]+TX, epsilon-momfluorothrin [1065124-65-3]+TX, pydiflumetofen [1228284-64-7]+TX, kappa-bifenthrin [439680-76-9]+TX, broflanilide [1207727-04-5]+TX, dicloromezotiaz [1263629-39-5]+TX, dipymetitrone [16114-35-5]+TX, pyraziflumid [942515-63-1]+TX and kappa-tefluthrin [391634-71-2]+TX; and

microbials including: Acinetobacter lwoffii+TX, Acremonium alternatum+TX+TX, Acremonium cephalosporium+TX+TX, Acremonium diospyri+TX, Acremonium obclavatum+TX, Adoxophyes orana granulovirus (AdoxGV) (Capex®)+TX, Agrobacterium radiobacter strain K84 (Galltrol-A®)+TX, Alternaria alternate+TX, Alternaria cassia+TX, Alternaria destruens (Smolder®)+TX, Ampelomyces quisqualis (AQ10®)+TX, Aspergillus flavus AF36 (AF36®)+TX, Aspergillus flavus NRRL 21882 (Aflaguard®)+TX, Aspergillus spp.+TX, Aureobasidium pullulans+TX, Azospirillum+TX, (MicroAZ®+TX, TAZO B®)+TX, Azotobacter+TX, Azotobacter chroocuccum (Azotomeal®)+TX, Azotobacter cysts (Bionatural Blooming Blossoms®)+TX, Bacillus amyloliquefaciens+TX, Bacillus cereus+TX, Bacillus chitinosporus strain CM-1+TX, Bacillus chitinosporus strain AQ746+TX, Bacillus licheniformis strain HB-2 (Biostart™ Rhizoboost®)+TX, Bacillus licheniformis strain 3086 (EcoGuard®+TX, Green Releaf®)+TX, Bacillus circulans+TX, Bacillus firmus (BioSafe®+TX, BioNem-WP®+TX, VOTiVO®)+TX, Bacillus firmus strain 1-1582+TX, Bacillus macerans+TX, Bacillus marismortui+TX, Bacillus megaterium+TX, Bacillus mycoides strain AQ726+TX, Bacillus papillae (Milky Spore Powder®)+TX, Bacillus pumilus spp.+TX, Bacillus pumilus strain GB34 (Yield Shield®)+TX, Bacillus pumilus strain AQ717+TX, Bacillus pumilus strain QST 2808 (Sonata®+TX, Ballad Plus®)+TX, Bacillus spahericus (VectoLex®)+TX, Bacillus spp.+TX, Bacillus spp. strain AQ175+TX, Bacillus spp. strain AQ177+TX, Bacillus spp. strain AQ178+TX, Bacillus subtilis strain QST 713 (CEASE®+TX, Serenade®+TX, Rhapsody®)+TX, Bacillus subtilis strain QST 714 (JAZZ®)+TX, Bacillus subtilis strain AQ153+TX, Bacillus subtilis strain AQ743+TX, Bacillus subtilis strain QST3002+TX, Bacillus subtilis strain QST3004+TX, Bacillus subtilis var. amyloliquefaciens strain FZB24 (Taegro®+TX, Rhizopro®)+TX, Bacillus thuringiensis Cry 2Ae+TX, Bacillus thuringiensis Cry1Ab+TX, Bacillus thuringiensis aizawai GC 91 (Agree®)+TX, Bacillus thuringiensis israelensis (BMP123®+TX, Aquabac®+TX, VectoBac®)+TX, Bacillus thuringiensis kurstaki (Javelin®+TX, Deliver®+TX, CryMax®+TX, Bonide®+TX, Scutella WP®+TX, Turilav WP®+TX, Astuto®+TX, Dipel WP®+TX, Biobit®+TX, Foray®)+TX, Bacillus thuringiensis kurstaki BMP 123 (Baritone®)+TX, Bacillus thuringiensis kurstaki HD-1 (Bioprotec-CAF/3P®)+TX, Bacillus thuringiensis strain BD#32+TX, Bacillus thuringiensis strain AQ52+TX, Bacillus thuringiensis var. aizawai (XenTari®+TX, DiPel®)+TX, bacteria spp. (GROWMEND®+TX, GROWSWEET®+TX, Shootup®)+TX, bacteriophage of Clavipacter michiganensis (AgriPhage®)+TX, Bakflor®+TX, Beauveria bassiana (Beaugenic®+TX, Brocaril WP®)+TX, Beauveria bassiana GHA (Mycotrol ES®+TX, Mycotrol O®+TX, BotaniGuard®)+TX, Beauveria brongniartii (Engerlingspilz®+TX, Schweizer Beauveria®+TX, Melocont®)+TX, Beauveria spp.+TX, Botrytis cineria+TX, Bradyrhizobium japonicum (TerraMax®)+TX, Brevibacillus brevis+TX, Bacillus thuringiensis tenebrionis (Novodor®)+TX, BtBooster+TX, Burkholderia cepacia (Deny®+TX, Intercept®+TX, Blue Circle®)+TX, Burkholderia gladii+TX, Burkholderia gladioli+TX, Burkholderia spp.+TX, Canadian thistle fungus (CBH Canadian Bioherbicide®)+TX, Candida butyri+TX, Candida famata+TX, Candida fructus+TX, Candida glabrata+TX, Candida guilliermondii+TX, Candida melibiosica+TX, Candida oleophila strain 0+TX, Candida parapsilosis+TX, Candida pelliculosa+TX, Candida pulcherrima+TX, Candida reukaufii+TX, Candida saitoana (Bio-Coat®+TX, Biocure®)+TX, Candida sake+TX, Candida spp.+TX, Candida tenius+TX, Cedecea dravisae+TX, Cellulomonas flavigena+TX, Chaetomium cochliodes (Nova-Cide®)+TX, Chaetomium globosum (Nova-Cide®)+TX, Chromobacterium subtsugae strain PRAA4-1T (Grandevo®)+TX, Cladosporium cladosporioides+TX, Cladosporium oxysporum+TX, Cladosporium chlorocephalum+TX, Cladosporium spp.+TX, Cladosporium tenuissimum+TX, Clonostachys rosea (EndoFine®)+TX, Colletotrichum acutatum+TX, Coniothyrium minitans (Cotans WG®)+TX, Coniothyrium spp.+TX, Cryptococcus albidus (YIELDPLUS®)+TX, Cryptococcus humicola+TX, Cryptococcus infirmo-miniatus+TX, Cryptococcus laurentii+TX, Cryptophlebia leucotreta granulovirus (Cryptex®)+TX, Cupriavidus campinensis+TX, Cydia pomonella granulovirus (CYD-X®)+TX, Cydia pomonella granulovirus (Madex®+TX, Madex Plus®+TX, Madex Max/Carpovirusine®)+TX, Cylindrobasidium laeve (Stumpout®)+TX, Cylindrocladium+TX, Debaryomyces hansenii+TX, Drechslera hawaiinensis+TX, Enterobacter cloacae+TX, Enterobacteriaceae+TX, Entomophtora virulenta (Vektor®)+TX, Epicoccum nigrum+TX, Epicoccum purpurascens+TX, Epicoccum spp.+TX, Filobasidium floriforme+TX, Fusarium acuminatum+TX, Fusarium chlamydosporum+TX, Fusarium oxysporum (Fusaclean®/Biofox C®)+TX, Fusarium proliferatum+TX, Fusarium spp.+TX, Galactomyces geotrichum+TX, Gliocladium catenulatum (Primastop®+TX, Prestop®)+TX, Gliocladium roseum+TX, Gliocladium spp. (SoilGard®)+TX, Gliocladium virens (Soilgard®)+TX, Granulovirus (Granupom®)+TX, Halobacillus halophilus+TX, Halobacillus litoralis+TX, Halobacillus trueperi+TX, Halomonas spp.+TX, Halomonas subglaciescola+TX, Halovibrio variabilis+TX, Hanseniaspora uvarum+TX, Helicoverpa armigera nucleopolyhedrovirus (Helicovex®)+TX, Helicoverpa zea nuclear polyhedrosis virus (Gemstar®)+TX, Isoflavone—formononetin (Myconate®)+TX, Kloeckera apiculata+TX, Kloeckera spp.+TX, Lagenidium giganteum (Laginex®)+TX, Lecanicillium longisporum (Vertiblast®)+TX, Lecanicillium muscarium (Vertikil®)+TX, Lymantria Dispar nucleopolyhedrosis virus (Disparvirus®)+TX, Marinococcus halophilus+TX, Meira geulakonigii+TX, Metarhizium anisopliae (Met52®)+TX, Metarhizium anisopliae (Destruxin WP®)+TX, Metschnikowia fruticola (Shemer®)+TX, Metschnikowia pulcherrima+TX, Microdochium dimerum (Antibot®)+TX, Micromonospora coerulea+TX, Microsphaeropsis ochracea+TX, Muscodor albus 620 (Muscudor®)+TX, Muscodor roseus strain A3-5+TX, Mycorrhizae spp. (AMykor®+TX, Root Maximizer®)+TX, Myrothecium verrucaria strain AARC-0255 (DiTera®)+TX, BROS PLUS®+TX, Ophiostoma piliferum strain D97 (Sylvanex®)+TX, Paecilomyces farinosus+TX, Paecilomyces fumosoroseus (PFR-97®+TX, PreFeRal®)+TX, Paecilomyces linacinus (Biostat WP®)+TX, Paecilomyces lilacinus strain 251 (MeloCon WG®)+TX, Paenibacillus polymyxa+TX, Pantoea agglomerans (BlightBan C9-1®)+TX, Pantoea spp.+TX, Pasteuria spp. (Econem®)+TX, Pasteuria nishizawae+TX, Penicillium aurantiogriseum+TX, Penicillium billai (Jumpstart®+TX, TagTeam®)+TX, Penicillium brevicompactum+TX, Penicillium frequentans+TX, Penicillium griseofulvum+TX, Penicillium purpurogenum+TX, Penicillium spp.+TX, Penicillium viridicatum+TX, Phlebiopsis gigantean (Rotstop®)+TX, phosphate solubilizing bacteria (Phosphomeal®)+TX, Phytophthora cryptogea+TX, Phytophthora palmivora (Devine®)+TX, Pichia anomala+TX, Pichia guilermondii+TX, Pichia membranaefaciens+TX, Pichia onychis+TX, Pichia stipites+TX, Pseudomonas aeruginosa+TX, Pseudomonas aureofasciens (Spot-Less Biofungicide®)+TX, Pseudomonas cepacia+TX, Pseudomonas chlororaphis (AtEze®)+TX, Pseudomonas corrugate+TX, Pseudomonas fluorescens strain A506 (BlightBan A506®)+TX, Pseudomonas putida+TX, Pseudomonas reactans+TX, Pseudomonas spp.+TX, Pseudomonas syringae (Bio-Save®)+TX, Pseudomonas viridiflava+TX, Pseudomons fluorescens (Zequanox®)+TX, Pseudozyma flocculosa strain PF-A22 UL (Sporodex L®)+TX, Puccinia canaliculata+TX, Puccinia thlaspeos (Wood Warrior®)+TX, Pythium paroecandrum+TX, Pythium oligandrum (Polygandron®+TX, Polyversum®)+TX, Pythium periplocum+TX, Rhanella aquatilis+TX, Rhanella spp.+TX, Rhizobia (Dormal®+TX, Vault®)+TX, Rhizoctonia+TX, Rhodococcus globerulus strain AQ719+TX, Rhodosporidium diobovatum+TX, Rhodosporidium toruloides+TX, Rhodotorula spp.+TX, Rhodotorula glutinis+TX, Rhodotorula graminis+TX, Rhodotorula mucilagnosa+TX, Rhodotorula rubra+TX, Saccharomyces cerevisiae+TX, Salinococcus roseus+TX, Sclerotinia minor+TX, Sclerotinia minor (SARRITOR®)+TX, Scytalidium spp.+TX, Scytalidium uredinicola+TX, Spodoptera exigua nuclear polyhedrosis virus (Spod-X®+TX, Spexit®)+TX, Serratia marcescens+TX, Serratia plymuthica+TX, Serratia spp.+TX, Sordaria fimicola+TX, Spodoptera littoralis nucleopolyhedrovirus (Littovir®)+TX, Sporobolomyces roseus+TX, Stenotrophomonas maltophilia+TX, Streptomyces ahygroscopicus+TX, Streptomyces albaduncus+TX, Streptomyces exfoliates+TX, Streptomyces galbus+TX, Streptomyces griseoplanus+TX, Streptomyces griseoviridis (Mycostop®)+TX, Streptomyces lydicus (Actinovate®)+TX, Streptomyces lydicus WYEC-108 (ActinoGrow®)+TX, Streptomyces violaceus+TX, Tilletiopsis minor+TX, Tilletiopsis spp.+TX, Trichoderma asperellum (T34 Biocontrol®)+TX, Trichoderma gamsii (Tenet®)+TX, Trichoderma atroviride (Plantmate®)+TX, Trichoderma hamatum TH 382+TX, Trichoderma harzianum rifai (Mycostar®)+TX, Trichoderma harzianum T-22 (Trianum-P®+TX, PlantShield HC®+TX, RootShield®+TX, Trianum-G®)+TX, Trichoderma harzianum T-39 (Trichodex®)+TX, Trichoderma inhamatum+TX, Trichoderma koningii+TX, Trichoderma spp. LC 52 (Sentinel®)+TX, Trichoderma lignorum+TX, Trichoderma longibrachiatum+TX, Trichoderma polysporum (Binab T®)+TX, Trichoderma taxi+TX, Trichoderma virens+TX, Trichoderma virens (formerly Gliocladium virens GL-21) (SoilGuard®)+TX, Trichoderma viride+TX, Trichoderma viride strain ICC 080 (Remedier®)+TX, Trichosporon pullulans+TX, Trichosporon spp.+TX, Trichothecium spp.+TX, Trichothecium roseum+TX, Typhula phacorrhiza strain 94670+TX, Typhula phacorrhiza strain 94671+TX, Ulocladium atrum+TX, Ulocladium oudemansii (Botry-Zen®)+TX, Ustilago maydis+TX, various bacteria and supplementary micronutrients (Natural II®)+TX, various fungi (Millennium Microbes®)+TX, Verticillium chlamydosporium+TX, Verticillium lecanii (Mycotal®+TX, Vertalec®)+TX, Vip3Aa20 (VIPtera®)+TX, Virgibaclillus marismortui+TX, Xanthomonas campestris pv. Poae (Camperico®)+TX, Xenorhabdus bovienii+TX, Xenorhabdus nematophilus; and

Plant extracts including: pine oil (Retenol®)+TX, azadirachtin (Plasma Neem Oil®+TX, AzaGuard®+TX, MeemAzal®+TX, Molt-X®+TX, Botanical IGR (Neemazad®+TX, Neemix®)+TX, canola oil (Lilly Miller Vegol®)+TX, Chenopodium ambrosioides near ambrosioides (Requiem®)+TX, Chrysanthemum extract (Crisant®)+TX, extract of neem oil (Trilogy®)+TX, essentials oils of Labiatae (Botania®)+TX, extracts of clove rosemary peppermint and thyme oil (Garden Insect Killer®)+TX, Glycinebetaine (Greenstim®)+TX, garlic+TX, lemongrass oil (GreenMatch®)+TX, neem oil+TX, Nepeta cataria (Catnip oil)+TX, Nepeta catarina+TX, nicotine+TX, oregano oil (MossBuster®)+TX, Pedaliaceae oil (Nematon®)+TX, pyrethrum+TX, Quillaja saponaria (NemaQ®)+TX, Reynoutria sachalinensis (Regalia®+TX, Sakalia®)+TX, rotenone (Eco Roten®)+TX, Rutaceae plant extract (Soleo®)+TX, soybean oil (Ortho Ecosense®)+TX, tea tree oil (Timorex Gold®)+TX, thymus oil+TX, AGNIQUE® MMF+TX, BugOil®+TX, mixture of rosemary sesame peppermint thyme and cinnamon extracts (EF 300®)+TX, mixture of clove rosemary and peppermint extract (EF 400®)+TX, mixture of clove peppermint garlic oil and mint (Soil Shot®)+TX, kaolin (Screen®)+TX, storage glucam of brown algae (Laminarin®); and

pheromones including: blackheaded fireworm pheromone (3M Sprayable Blackheaded Fireworm Pheromone®)+TX, Codling Moth Pheromone (Paramount dispenser-(CM)/Isomate C-Plus®)+TX, Grape Berry Moth Pheromone (3M MEC-GBM Sprayable Pheromone®)+TX, Leafroller pheromone (3M MEC—LR Sprayable Pheromone®)+TX, Muscamone (Snip7 Fly Bait®+TX, Starbar Premium Fly Bait®)+TX, Oriental Fruit Moth Pheromone (3M Oriental Fruit Moth Sprayable Pheromone®)+TX, Peachtree Borer Pheromone (Isomate-P®)+TX, Tomato Pinworm Pheromone (3M Sprayable Pheromone®)+TX, Entostat powder (extract from palm tree) (Exosex CM®)+TX, (E+TX,Z+TX,Z)-3+TX,8+TX,11 Tetradecatrienyl acetate+TX, (Z+TX,Z+TX,E)-7+TX,11+TX,13-Hexadecatrienal+TX, (E+TX,Z)-7+TX,9-Dodecadien-1-yl acetate+TX, 2-Methyl-1-butanol+TX, Calcium acetate+TX, Scenturion®+TX, Biolure®+TX, Check-Mate®+TX, Lavandulyl senecioate; and

Macrobials including: Aphelinus abdominalis+TX, Aphidius ervi (Aphelinus-System®+TX, Acerophagus papaya+TX, Adalia bipunctata (Adalia-System®)+TX, Adalia bipunctata (Adaline®)+TX, Adalia bipunctata (Aphidalia®)+TX, Ageniaspis citricola+TX, Ageniaspis fuscicollis+TX, Amblyseius andersoni (Anderline®+TX, Andersoni-System®)+TX, Amblyseius califomicus (Amblyline®+TX, Spical®)+TX, Amblyseius cucumeris (Thripex®+TX, Bugline Cucumeris®)+TX, Amblyseius fallacis (Fallacis®)+TX, Amblyseius swirskii (Bugline Swirskii®+TX, Swirskii-Mite®)+TX, Amblyseius womersleyi (WomerMite®)+TX, Amitus hesperidum+TX, Anagrus atomus+TX, Anagyrus fusciventris+TX, Anagyrus kamali+TX, Anagyrus loecki+TX, Anagyrus pseudococci (Citripar®)+TX, Anicetus benefices+TX, Anisopteromalus calandrae+TX, Anthocoris nemoralis (Anthocoris-System®)+TX, Aphelinus abdominalis (Apheline®+TX, Aphiline®)+TX, Aphelinus asychis+TX, Aphidius colemani (Aphipar®)+TX, Aphidius ervi (Ervipar®)+TX, Aphidius gifuensis+TX, Aphidius matricariae (Aphipar-M®)+TX, Aphidoletes aphidimyza (Aphidend®)+TX, Aphidoletes aphidimyza (Aphidoline®)+TX, Aphytis lingnanensis+TX, Aphytis melinus+TX, Aprostocetus hagenowii+TX, Atheta coriaria (Staphyline®)+TX, Bombus spp.+TX, Bombus terrestris (Natupol Beehive®)+TX, Bombus terrestris (Beeline®+TX, Tripol®)+TX, Cephalonomia stephanoderis+TX, Chilocorus nigritus+TX, Chrysoperla carnea (Chrysoline®)+TX, Chrysoperla carnea (Chrysopa®)+TX, Chrysoperla rufilabris+TX, Cirrospilus ingenuus+TX, Cirrospilus quadristriatus+TX, Citrostichus phyllocnistoides+TX, Closterocerus chamaeleon+TX, Closterocerus spp.+TX, Coccidoxenoides perminutus (Planopar®)+TX, Coccophagus cowperi+TX, Coccophagus lycimnia+TX, Cotesia flavipes+TX, Cotesia plutellae+TX, Cryptolaemus montrouzieri (Cryptobug®+TX, Cryptoline®)+TX, Cybocephalus nipponicus+TX, Dacnusa sibirica+TX, Dacnusa sibirica (Minusa®)+TX, Diglyphus isaea (Diminex®)+TX, Delphastus catalinae (Delphastus®)+TX, Delphastus pusillus+TX, Diachasmimorpha krausii+TX, Diachasmimorpha longicaudata+TX, Diaparsis jucunda+TX, Diaphorencyrtus aligarhensis+TX, Diglyphus isaea+TX, Diglyphus isaea (Miglyphus®+TX, Digline®)+TX, Dacnusa sibirica (DacDigline®+TX, Minex®)+TX, Diversinervus spp.+TX, Encarsia citrina+TX, Encarsia formosa (Encarsia Max®+TX, Encarline®+TX, En-Strip®)+TX, Eretmocerus eremicus (Enermix®)+TX, Encarsia guadeloupae+TX, Encarsia haitiensis+TX, Episyrphus balteatus (Syrphidend®)+TX, Eretmoceris siphonini+TX, Eretmocerus califomicus+TX, Eretmocerus eremicus (Ercal®+TX, Eretline e®)+TX, Eretmocerus eremicus (Bemimix®)+TX, Eretmocerus hayati+TX, Eretmocerus mundus (Bemipar®+TX, Eretline m®)+TX, Eretmocerus siphonini+TX, Exochomus quadripustulatus+TX, Feltiella acarisuga (Spidend®)+TX, Feltiella acarisuga (Feltiline®)+TX, Fopius arisanus+TX, Fopius ceratitivorus+TX, Formononetin (Wirless Beehome®)+TX, Franklinothrips vespiformis (Vespop®)+TX, Galendromus occidentalis+TX, Goniozus legneri+TX, Habrobracon hebetor+TX, Harmonia axyridis (HarmoBeetle®)+TX, Heterorhabditis spp. (Lawn Patrol®)+TX, Heterorhabditis bacteriophora (NemaShield HB®+TX, Nemaseek®+TX, Terranem-Nam®+TX, Terranem®+TX, Larvanem®+TX, B-Green®+TX, NemAttack®+TX, Nematop®)+TX, Heterorhabditis megidis (Nemasys H®+TX, BioNem H®+TX, Exhibitline hm®+TX, Larvanem-M®)+TX, Hippodamia convergens+TX, Hypoaspis aculeifer (Aculeifer-System®+TX, Entomite-A®)+TX, Hypoaspis miles (Hypoline m®+TX, Entomite-M®)+TX, Lbalia leucospoides+TX, Lecanoideus floccissimus+TX, Lemophagus errabundus+TX, Leptomastidea abnormis+TX, Leptomastix dactylopii (Leptopar®)+TX, Leptomastix epona+TX, Lindorus lophanthae+TX, Lipolexis oregmae+TX, Lucilia caesar (Natufly®)+TX, Lysiphlebus testaceipes+TX, Macrolophus caliginosus (Mirical-N®+TX, Macroline c®+TX, Mirical®)+TX, Mesoseiulus longipes+TX, Metaphycus flavus+TX, Metaphycus lounsburyi+TX, Micromus angulatus (Milacewing®)+TX, Microterys flavus+TX, Muscidifurax raptorellus and Spalangia cameroni (Biopar®)+TX, Neodryinus typhlocybae+TX, Neoseiulus californicus+TX, Neoseiulus cucumeris (THRYPEX®)+TX, Neoseiulus fallacis+TX, Nesideocoris tenuis (NesidioBug®+TX, Nesibug®)+TX, Ophyra aenescens (Biofly®)+TX, Orius insidiosus (Thripor-I®+TX, Oriline i®)+TX, Orius laevigatus (Thripor-L®+TX, Oriline I®)+TX, Orius majusculus (Oriline m®)+TX, Orius strigicollis (Thripor-S®)+TX, Pauesia juniperorum+TX, Pediobius foveolatus+TX, Phasmarhabditis hermaphrodita (Nemaslug®)+TX, Phymastichus coffea+TX, Phytoseiulus macropilus+TX, Phytoseiulus persimilis (Spidex®+TX, Phytoline p®)+TX, Podisus maculiventris (Podisus®)+TX, Pseudacteon curvatus+TX, Pseudacteon obtusus+TX, Pseudacteon tricuspis+TX, Pseudaphycus maculipennis+TX, Pseudleptomastix mexicana+TX, Psyllaephagus pilosus+TX, Psyttalia concolor (complex)+TX, Quadrastichus spp.+TX, Rhyzobius lophanthae+TX, Rodolia cardinalis+TX, Rumina decollate+TX, Semielacher petiolatus+TX, Sitobion avenae (Ervibank®)+TX, Steinernema carpocapsae (Nematac C®+TX, Millenium®+TX, BioNem C®+TX, NemAttack®+TX, Nemastar®+TX, Capsanem®)+TX, Steinernema feltiae (NemaShield®+TX, Nemasys F®+TX, BioNem F®+TX, Steinernema-System®+TX, NemAttack®+TX, Nemaplus®+TX, Exhibitline sf®+TX, Scia-Rid®+TX, Entonem®)+TX, Steinernema kraussei (Nemasys L®+TX, BioNem L®+TX, Exhibitline srb®)+TX, Steinernema riobrave (BioVector®+TX, BioVektor®)+TX, Steinernema scapterisci (Nematac S®)+TX, Steinernema spp.+TX, Steinernematid spp. (Guardian Nematodes®)+TX, Stethorus punctillum (Stethorus®)+TX, Tamarixia radiate+TX, Tetrastichus setifer+TX, Thripobius semiluteus+TX, Torymus sinensis+TX, Trichogramma brassicae (Tricholine b®)+TX, Trichogramma brassicae (Tricho-Strip®)+TX, Trichogramma evanescens+TX, Trichogramma minutum+TX, Trichogramma ostriniae+TX, Trichogramma platneri+TX, Trichogramma pretiosum+TX, Xanthopimpla stemmator; and

other biologicals including: abscisic acid+TX, bioSea®+TX, Chondrostereum purpureum (Chontrol Paste®)+TX, Colletotrichum gloeosporioides (Gallego®)+TX, Copper Octanoate (Cueva®)+TX, Delta traps (Trapline d®)+TX, Erwinia amylovora (Harpin) (ProAct®+TX, Ni-HIBIT Gold CST®)+TX, Ferri-phosphate (Ferramol®)+TX, Funnel traps (Trapline y®)+TX, Gallex®+TX, Growers Secret®+TX, Homo-brassonolide+TX, Iron Phosphate (Lilly Miller Worry Free Ferramol Slug & Snail Bait®)+TX, MCP hail trap (Trapline f®)+TX, Microctonus hyperodae+TX, Mycoleptodiscus terrestris (Des-X®)+TX, BioGain®+TX, Aminomite®+TX, Zenox®+TX, Pheromone trap (Thripline ams®)+TX, potassium bicarbonate (MilStop®)+TX, potassium salts of fatty acids (Sanova®)+TX, potassium silicate solution (SD-Matrix®)+TX, potassium iodide+potassiumthiocyanate (Enzicur®)+TX, SuffOil-X®+TX, Spider venom+TX, Nosema locustae (Semaspore Organic Grasshopper Control®)+TX, Sticky traps (Trapline YF®+TX, Rebell Amarillo®)+TX and Traps (Takitrapline y+b®)+TX;

or a biologically active compound or agent selected from: Brofluthrinate+TX, Diflovidazine+TX, Flometoquin+TX, Fluhexafon+TX, Plutella xylostella Granulosis virus+TX, Cydia pomonella Granulosis virus+TX, Imicyafos+TX, Heliothis virescens Nucleopolyhedrovirus+TX, Heliothis punctigera Nucleopolyhedrovirus+TX, Helicoverpa zea Nucleopolyhedrovirus+TX, Spodoptera frugiperda Nucleopolyhedrovirus+TX, Plutella xylostella Nucleopolyhedrovirus+TX, p-cymene+TX, Pyflubumide+TX, Pyrafluprole+TX, QRD 420+TX, QRD 452+TX, QRD 460+TX, Terpenoid blends+TX, Terpenoids+TX, Tetraniliprole+TX, and α-terpinene+TX;

or an active substance referenced by a code+TX, such as code AE 1887196 (BSC-BX60309)+TX, code NNI-0745 GR+TX, code IKI-3106+TX, code JT-L001+TX, code ZNQ-08056+TX, code IPPA152201+TX, code HNPC-A9908 (CAS: [660411-21-2])+TX, code HNPC-A2005 (CAS: [860028-12-2])+TX, code JS118+TX, code ZJ0967+TX, code ZJ2242+TX, code JS7119 (CAS: [929545-74-4])+TX, code SN-1172+TX, code HNPC-A9835+TX, code HNPC-A9955+TX, code HNPC-A3061+TX, code Chuanhua 89-1+TX, code IPP-10+TX, code ZJ3265+TX, code JS9117+TX, code ZJ3757+TX, code ZJ4042+TX, code ZJ4014+TX, code ITM-121+TX, code DPX-RAB55 (DKI-2301)+TX, code NA-89+TX, code MIE-1209+TX, code MCI-8007+TX, code BCS-CL73507+TX, code S-1871+TX, code DPX-RDS63+TX, Quinofumelin+TX, mefentrifluconazol+TX, fenpicoxamid+TX, fluindapyr+TX, inpyrfluxam+TX or indiflumetpyr+TX, isoflucypram+TX, pyrapropoyne+TX, florylpicoxamid+TX, metyltetraprole+TX, ipflufenoquin+TX, pyridachlometyl+TX or chlopyridiflu+TX, tetrachlorantraniliprole+TX, tetrachloraniliprole+TX, Tyclopyrazoflor+TX, flupyrimin+TX or pyrifluramide+TX, benzpyrimoxan+TX, Benzosufyl+TX or oxazosulfyl+TX, etpyrafen+TX, acynonapyr+TX or pyrinonafen+TX, oxotrione+TX, bixlozone+TX or clofendizone+TX or dicloroxizone+TX, cyclopyranil+TX or pyrazocyclonil+TX or cyclopyrazonil+TX, alpha-bromadiolone+TX,

and code AKD-1193+TX.

The references in brackets behind the active ingredients, e.g. [3878-19-1] refer to the Chemical Abstracts Registry number. The above described mixing partners are known. Where the active ingredients are included in “The Pesticide Manual” [The Pesticide Manual—A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright® 1995-2004]; for example, the compound “acetoprole” is described under the internet address http://www.alanwood.net/pesticides/acetoprole.html.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “develoment code” is used or, if neither one of those designations nor a “common name” is used, an “alternative name” is employed. “CAS Reg. No” means the Chemical Abstracts Registry Number.

The active ingredient mixture of the compounds of formula I selected from Tables 1-3 and P with active ingredients described above comprises a compound selected from Tables 1-3 and P and an active ingredient as described above preferably in a mixing ratio of from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight.

The mixtures as described above can be used in a method for controlling pests, which comprises applying a composition comprising a mixture as described above to the pests or their environment, with the exception of a method for treatment of the human or animal body by surgery or therapy and diagnostic methods practised on the human or animal body.

The mixtures comprising a compound of formula I selected from Tables 1-3 and P and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula I selected from Tables 1-3 and P and the active ingredients as described above is not essential for working the present invention.

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds I for the preparation of these compositions are also a subject of the invention.

The application methods for the compositions, that is the methods of controlling pests of the abovementioned type, such as spraying, atomizing, dusting, brushing on, dressing, scattering or pouring—which are to be selected to suit the intended aims of the prevailing circumstances—and the use of the compositions for controlling pests of the abovementioned type are other subjects of the invention. Typical rates of concentration are between 0.1 and 1000 ppm, preferably between 0.1 and 500 ppm, of active ingredient. The rate of application per hectare is generally 1 to 2000 g of active ingredient per hectare, in particular 10 to 1000 g/ha, preferably 10 to 600 g/ha.

A preferred method of application in the field of crop protection is application to the foliage of the plants (foliar application), it being possible to select frequency and rate of application to match the danger of infestation with the pest in question. Alternatively, the active ingredient can reach the plants via the root system (systemic action), by drenching the locus of the plants with a liquid composition or by incorporating the active ingredient in solid form into the locus of the plants, for example into the soil, for example in the form of granules (soil application). In the case of paddy rice crops, such granules can be metered into the flooded paddy-field.

The compounds of the invention and compositions thereof are also be suitable for the protection of plant propagation material, for example seeds, such as fruit, tubers or kernels, or nursery plants, against pests of the abovementioned type. The propagation material can be treated with the compound prior to planting, for example seed can be treated prior to sowing. Alternatively, the compound can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention. Typical treatment rates would depend on the plant and pest/fungi to be controlled and are generally between 1 to 200 grams per 100 kg of seeds, preferably between 5 to 150 grams per 100 kg of seeds, such as between 10 to 100 grams per 100 kg of seeds.

The term seed embraces seeds and plant propagules of all kinds including but not limited to true seeds, seed pieces, suckers, corns, bulbs, fruit, tubers, grains, rhizomes, cuttings, cut shoots and the like and means in a preferred embodiment true seeds.

The present invention also comprises seeds coated or treated with or containing a compound of formula I. The term “coated or treated with and/or containing” generally signifies that the active ingredient is for the most part on the surface of the seed at the time of application, although a greater or lesser part of the ingredient may penetrate into the seed material, depending on the method of application. When the said seed product is (re)planted, it may absorb the active ingredient. In an embodiment, the present invention makes available a plant propagation material adhered thereto with a compound of formula (I). Further, it is hereby made available, a composition comprising a plant propagation material treated with a compound of formula (I).

Seed treatment comprises all suitable seed treatment techniques known in the art, such as seed dressing, seed coating, seed dusting, seed soaking and seed pelleting. The seed treatment application of the compound formula (I) can be carried out by any known methods, such as spraying or by dusting the seeds before sowing or during the sowing/planting of the seeds.

### Biological Examples

### Example Bemisia tabaci (Cotton White Fly): Feeding/Contact Activity

Cotton leaf discs were placed on agar in 24-well microtiter plates and sprayed with aqueous test solutions prepared from 10′000 ppm DMSO stock solutions. After drying the leaf discs were infested with adult white flies. The samples were checked for mortality 6 days after incubation.

The following compounds resulted in at least 80% mortality at an application rate of 200 ppm:

P3, P5, P8, P9, P10, P11, P15, P16, P18, P19, P26, P28, P30, P40, P41, P42, P44, P46, P47, P50, P52, P53, P56, P60, P61, P62, P67, P70, P71, P72, P75, P76, P78, P79, P89, P97, P100, P101, P103, P104, P105, P106

### Example B2: Diabrotica Balteata (Corn Root Worm)

Maize sprouts placed onto an agar layer in 24-well microtiter plates were treated with aqueous test solutions prepared from 10′000 ppm DMSO stock solutions by spraying. After drying, the plates were infested with L2 larvae (6 to 10 per well). The samples were assessed for mortality and growth inhibition in comparison to untreated samples 4 days after infestation.

The following compounds gave an effect of at least 80% in at least one of the two categories (mortality or growth inhibition) at an application rate of 200 ppm:

P1, P2, P3, P4, P5, P6, P8, P9, P10, P12, P13, P14, P15, P16, P17, P18, P19, P22, P23, P24, P26, P27, P28, P30, P31, P40, P41, P42, P46, P47, P50, P53, P56, P61, P62, P65, P67, P68, P69, P71, P72, P73, P74, P75, P76, P78, P79, P81, P82, P83, P86, P89, P91, P94, P95, P96, P97, P98, P99, P100, P101, P103, P104, P105, P106, P107, P108

### Example B3: Euschistus heros(Neotropical Brown Stink Bug)

Soybean leaves on agar in 24-well microtiter plates were sprayed with aqueous test solutions prepared from 10′000 ppm DMSO stock solutions. After drying the leaves were infested with N2 nymphs. The samples were assessed for mortality and growth inhibition in comparison to untreated samples 5 days after infestation.

The following compounds gave an effect of at least 80% in at least one of the two categories (mortality or growth inhibition) at an application rate of 200 ppm:

P1, P2, P3, P4, P5, P6, P7, P8, P9, P10, P11, P15, P16, P18, P19, P22, P24, P28, P30, P31, P36, P37, P40,

P41, P42, P43, P44, P45, P46, P47, P49, P50, P51, P52, P53, P54, P56, P60, P62, P64, P65, P67, P68, P69, P70, P71, P72, P73, P74, P75, P76, P77, P78, P79, P80, P81, P82, P83, P85, P86, P87, P88, P89, P96, P97, P98, P99, P100, P101, P102, P103, P104, P105, P106, P107, P108

### Example B4: Frankliniella occidentalis (Western Flower Thrips):Feeding/Contact Activity

Sunflower leaf discs were placed on agar in 24-well microtiter plates and sprayed with aqueous test solutions prepared from 10′000 DMSO stock solutions. After drying the leaf discs were infested with a Frankliniella population of mixed ages. The samples were assessed for mortality 7 days after infestation.

The following compounds resulted in at least 80% mortality at an application rate of 200 ppm:

P10, P18, P41, P53, P60, P78, P103

### Example B5: Myzus persicae (Green Peach Aphid):Feeding/Contact Activity

Sunflower leaf discs were placed onto agar in a 24-well microtiter plate and sprayed with aqueous test solutions prepared from 10′000 ppm DMSO stock solutions. After drying, the leaf discs were infested with an aphid population of mixed ages. The samples were assessed for mortality 6 days after infestation.

The following compounds resulted in at least 80% mortality at an application rate of 200 ppm:

P1, P3, P4, P5, P6, P7, P8, P9, P10, P11, P12, P13, P15, P16, P18, P19, P22, P23, P24, P25, P26, P27, P28, P30, P31, P37, P40, P41, P42, P45, P46, P47, P48, P49, P50, P52, P53, P54, P55, P56, P60, P61, P62, P64, P65, P67, P68, P69, P70, P71, P72, P73, P74, P75, P76, P78, 7P9, P80, P85, P86, P87, P88, P89, P93, P97, 1P0, P101, P102, P103, P104, P105, P106, P107, P108

### Example B6: Myzus persicae (Green Peach Aphid). Systemic Activity

Roots of pea seedlings infested with an aphid population of mixed ages were placed directly into aqueous test solutions prepared from 10′000 DMSO stock solutions. The samples were assessed for mortality 6 days after placing seedlings into test solutions.

The following compounds resulted in at least 80% mortality at a test rate of 24 ppm:

P1, P4, P5, P6, P7, P8, P9, P10, P11, P12, P13, P15, P16, P18, P22, P24, P28, P30, P31, P38, P40, P41, P42, P45, P46, P47, P48, P49, P50, P51, P52, P53, P54, P55, P56, P59, P60, P65, P68, P70, P72, P75, P78, P79, P80, P81, P85, P86, P87, P88, P89, P100, P101, P102, P103, P104, P105, P106, P107

### Example B7: Myzus persicae (Green Peach Aphid). Intrinsic Activity

Test compounds prepared from 10′000 ppm DMSO stock solutions were applied by pipette into 24-well microtiter plates and mixed with sucrose solution. The plates were closed with a stretched Parafilm. A plastic stencil with 24 holes was placed onto the plate and infested pea seedlings were placed directly on the Parafilm. The infested plate was closed with a gel blotting paper and another plastic stencil and then turned upside down. The samples were assessed for mortality 5 days after infestation.

The following compounds resulted in at least 80% mortality at a test rate of 12 ppm:

P19

### Example B8: Plutella xylostella (Diamond Back Moth)

24-well microtiter plates with artificial diet were treated with aqueous test solutions prepared from 10′000 ppm DMSO stock solutions by pipetting. After drying, the plates were infested with L2 larvae (10 to 15 per well). The samples were assessed for mortality and growth inhibition in comparison to untreated samples 5 days after infestation.

The following compounds gave an effect of at least 80% in at least one of the two categories (mortality or growth inhibition) at an application rate of 200 ppm:

P1, P3, P4, P5, P6, P8, P12, P13, P14, P15, P16, P18, P19, P22, P23, P24, P25, P26, P27, P28, P30, P31, P40, P41, P42, P45, P46, P47, P48, P49, P50, P53, P60, P62, P64, P65, P67, P68, P70, P71, P72, P73, P74, P75, P76, P78, P79, P81, P83, P85, P86, P87, P88, P89, P93, P96, P97, P98, P99, P100, P101, P103, P107, P108, P109

### Example B9: Spodoptera littoralis (Egyptian Cotton Leaf Worm)

Cotton leaf discs were placed onto agar in 24-well microtiter plates and sprayed with aqueous test solutions prepared from 10′000 ppm DMSO stock solutions. After drying the leaf discs were infested with five L1 larvae. The samples were assessed for mortality, anti-feeding effect, and growth inhibition in comparison to untreated samples 3 days after infestation. Control of Spodoptera littoralis by a test sample is given when at least one of the categories mortality, anti-feedant effect, and growth inhibition is higher than the untreated sample.

The following compounds resulted in at least 80% control at an application rate of 200 ppm:

P5, P8, P10, P11, P12, P13, P15, P16, P18, P19, P22, P24, P26, P27, P28, P30, P31, P40, P41, P42, P45, P53, P60, P61, P62, P67, P68, P69, P70, P71, P72, P73, P74, P75, P76, P78, P79, P81, P82, P84, P85, P86, P87, P88, P92, P96, P97, P98, P99, P100, P101, P103, P107, P108

### Example B10: Spodoptera littoralis (Egyptian Cotton Leaf Worm)

Test compounds were applied by pipette from 10′000 ppm DMSO stock solutions into 24-well plates and mixed with agar. Lettuce seeds were placed onto the agar and the multi well plate was closed by another plate which contained also agar. After 7 days the compound was absorbed by the roots and the lettuce grew into the lid plate. The lettuce leaves were then cut off into the lid plate. Spodoptera eggs were pipetted through a plastic stencil onto a humid gel blotting paper and the lid plate was closed with it. The samples were assessed for mortality, anti-feedant effect and growth inhibition in comparison to untreated samples 6 days after infestation.

The following compounds gave an effect of at least 80% in at least one of the three categories (mortality, anti-feeding, or growth inhibition) at a test rate of 12.5 ppm:

P18, P24, P28, P97, P99

### Example B11: Tetranychus urticae (Two-Spotted Spider Mite):Feeding/Contact Activity

Bean leaf discs on agar in 24-well microtiter plates were sprayed with aqueous test solutions prepared from 10′000 ppm DMSO stock solutions. After drying the leaf discs were infested with a mite population of mixed ages. The samples were assessed for mortality on mixed population (mobile stages) 8 days after infestation.

The following compounds resulted in at least 80% mortality at an application rate of 200 ppm:

P5, P18, P19, P47, P53, P60, P62, P71, P76, P79, P87, P88, P99, P103, P104

1. A compound of formula I, 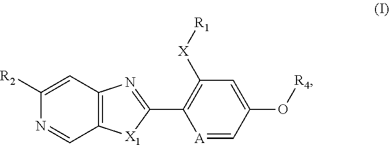
wherein
A is CH or N;
X is S, SO or SO₂;
R₁ is C₁-C₄alkyl, C₁-C₄haloalkyl or C₃-C₆cycloalkylC₁-C₄alkyl;
R₂ is halogen, C₁-C₆haloalkyl, C₁-C₄haloalkylsulfanyl, C₁-C₄haloalkylsulfinyl, C₁-C₄haloalkylsulfonyl or C₁-C₆haloalkoxy;
X₁ is O, S or NR₃, wherein R₃ is C₁-C₄alkyl;
R₄ is C₁-C₆alkyl, C₃-C₆cycloalkylC₁-C₄alkyl, C₃-C₆cyanocycloalkylC₁-C₄alkyl, C₃-C₆halocycloalkyl, C₃-C₆alkynyl, C₃-C₆haloalkenyl, C₃-C₆halocycloalkylC₁-C₄alkyl, C₁-C₄alkoxyC₁-C₄alkyl, C₁-C₄alkoximinoC₁-C₄alkyl, C₁-C₄alkylsulfanylC₁-C₄alkyl, C₁-C₄alkylsulfinylC₁-C₄alkyl, C₁-C₄alkylsulfonylC₁-C₄alkyl, C₁-C₆cyanoalkyl, aminocarbonylC₁-C₄alkyl, C₁-C₄alkylaminocarbonylC₁-C₄alkyl, di(C₁-C₄alkyl)aminocarbonylC₁-C₄alkyl, di(C₁-C₄alkyl)aminocarbonyl, hydroxycarbonylC₁-C₄alkyl, C₁-C₄alkoxycarbonylC₁-C₄alkyl, C₁-C₆cyanohaloalkyl, aminocarbonylC₁-C₄haloalkyl, C₁-C₄alkylaminocarbonylC₁-C₄haloalkyl, di(C₁-C₄alkyl)aminocarbonylC₁-C₄haloalkyl, hydroxycarbonylC₁-C₄haloalkyl, C₁-C₄alkoxycarbonylC₁-C₄haloalkyl, 2,2-difluoroethyl, 2,2,2-trifluoroethyl, 1,3-difluoropropyl, trifluoromethyl, 2-fluoro-1,1-dimethyl-ethyl, 2-fluoroethyl, 1-fluorethyl, 2,2-difluoropropyl, 2-fluoro-1-(fluoromethyl)ethyl, 2,2,2-trifluoro-1,1-dimethyl-ethyl, 1,1-difluoroethyl, di(C₁-C₄alkyl)aminocarbonylC₁-C₄haloalkyl, C₁-C₆cyano(C₁-C₄alkylsulfanyl)alkyl, C₁-C₄alkoxycarbonylC₁-C₄(C₁-C₄alkylsulfonyl)alkyl, C₁-C₄alkoxycarbonylC₁-C₄(C₁-C₄alkylsulfanyl)alkyl, aminocarbonylC₁-C₄(C₁-C₄alkylsulfanyl)alkyl, di(C₁-C₄alkyl)aminocarbonylC₁-C₄(C₁-C₄alkylsulfanyl)alkyl, C₁-C₄alkylaminocarbonylC₁-C₄(C₁-C₄alkylsulfanyl)alkyl, di(C₁-C₄alkyl)aminocarbonylC₁-C₄(C₁-C₄alkylsulfonyl)alkyl, tri-(C₁-C₆)alkylsilyl C₁-C₄alkyl, C₁-C₆alkylcarbonylC₁-C₄alkyl, imino-C₁-C₄alkyl-oxo-C₁-C₄alkyl-$l\hat{ }{6}-sulfane, C₁-C₄haloalkylsulfonyl, propan-2-imine; or
R₄ is C₃-C₆cycloalkyl which can be mono- or polysubstituted by substituents selected from the group consisting of halogen, cyano, C₁-C₄alkyl, C₁-C₄alkoxy and C₁-C₄haloalkyl; or
R₄ is phenyl or a four- to six-membered heterocyclic ring system which can be partially saturated or fully saturated, said ring system contains 1 to 2 heteroatoms selected from the group consisting of nitrogen, oxygen and sulfur, and said ring system can be mono- or polysubstituted by substituents selected from the group consisting of halogen, cyano, C₁-C₄alkyl, C₃-C₆cycloalkyl, C₁-C₄alkoxy, C₁-C₄haloalkyl and oxo, and where the ring system is attached directly to the oxygen or via an C₁-C₄alkylene chain;
and agrochemically acceptable salts, stereoisomers, enantiomers, tautomers and N-oxides of the compounds of formula I.

2. A compound of formula I according to claim 1, represented by the compounds of formula I-1 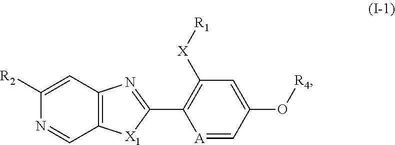
wherein A, X, R₁, R₂, and X₁ are as defined under formula I in claims 1 and R₄ is C₁-C₆alkyl, C₃-C₆cycloalkylC₁-C₄alkyl, C₃-C₆alkynyl, C₃-C₆haloalkenyl, C₃-C₆halocycloalkylC₁-C₄alkyl, C₁-C₄alkoxyC₁-C₄alkyl, C₁-C₄alkoximinoC₁-C₄alkyl, C₁-C₄alkylsulfanylC₁-C₄alkyl, C₁-C₄alkylsulfinylC₁-C₄alkyl, C₁-C₄alkylsulfonylC₁-C₄alkyl, C₁-C₆cyanoalkyl, aminocarbonylC₁-C₄alkyl, C₁-C₄alkylaminocarbonylC₁-C₄alkyl, di(C₁-C₄alkyl)aminocarbonylC₁-C₄alkyl, hydroxycarbonylC₁-C₄alkyl, C₁-C₄alkoxycarbonylC₁-C₄alkyl, C₁-C₆cyanohaloalkyl, aminocarbonylC₁-C₄haloalkyl, C₁-C₄alkylaminocarbonylC₁-C₄haloalkyl, di(C₁-C₄alkyl)aminocarbonylC₁-C₄haloalkyl, hydroxycarbonylC₁-C₄haloalkyl, C₁-C₄alkoxycarbonylC₁-C₄haloalkyl; or is C₃-C₆cycloalkyl which can be mono- or polysubstituted by substituents selected from the group consisting of halogen, cyano, C₁-C₄alkyl, C₁-C₄alkoxy, C₁-C₄haloalkyl, or R₄ is a four- to six-membered heterocyclic ring system which can be partially saturated or fully saturated, said ring system contains 1 to 2 heteroatoms selected from the group consisting of nitrogen, oxygen and sulfur, and said ring system can be mono- or polysubstituted by substituents selected from the group consisting of halogen, cyano, C₁-C₄alkyl, C₁-C₄alkoxy, C₁-C₄haloalkyl and oxo.

3. A compound of formula I according to claim 1, represented by the compounds of formula I-2 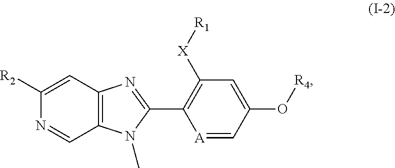
wherein A, X, R₁, and R₂ are defined under formula I in claims 1 and R₄ is defined as under formula I-1.
In said preferred group of compounds of formula I-2,
R₁ is preferably methyl, ethyl, n-propyl, i-propyl or cyclopropylmethyl;
R₂ is preferably C₁-C₄haloalkyl, C₁-C₄haloalkylsulfanyl, C₁-C₄haloalkylsulfinyl or C₁-C₄haloalkylsulfonyl;
and X is preferably SO₂.

4. A compound of formula I-2 according to claim 3, represented by the compounds of formula I-2a 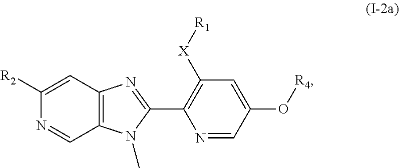
wherein X, R₁, and R₂ are defined under formula I in claim 1, and R₄ is defined as in claim 2.

5. A compound of formula I-2 according to claim 3, represented by the compounds of formula I-2b 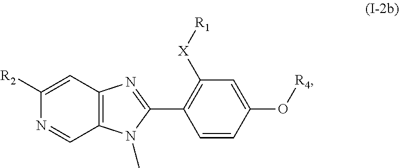
wherein X, R₁, and R₂ are defined under formula I in claims 1 and R₄ is defined as in claim 2. under formula I-1.

6. A compound of formula I according to claim 1, represented by the compounds of formula I-3 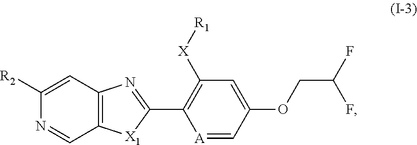
wherein A, X, X₁, R₁, and R₂ are as defined under formula I in claim 1.

7. A compound of formula I-3 according to claim 6, represented by the compounds of formula I-3a 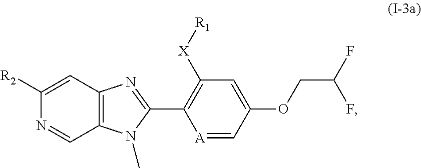
wherein A, X, R₁, and R₂ are as defined under formula I in claim 1.

8. A compound of formula I according to claim 1, represented by the compounds of formula I-4 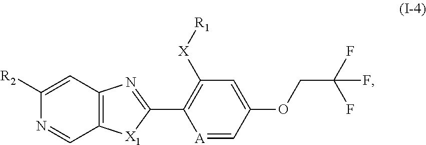
wherein A, X, X₁, R₁, and R₂ are as defined under formula I in claim 1.

9. A compound of formula I-4 according to claim 8, represented by the compounds of formula I-4a 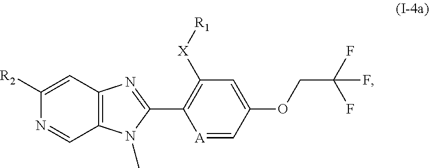
wherein A, X, R₁, and R₂ are as defined under formula I in claim 1.

10. A compound of formula I according to claim 1, wherein
R₁ is C₁-C₄alkyl;
R₂ is C₁-C₄haloalkyl;
X₁ is NR₃; wherein R₃ is C₁-C₄alkyl;
A is CH or N;
X is S or SO₂;
R₄ is oxetanyl which can be mono-substituted by C₁-C₄alkyl, or is C₁-C₄alkyl which can be substituted by cyano, or is C₃-C₆alkynyl, 2,2-difluoroethyl, 2,2,2-trifluoroethyl, C₃-C₆cycloalkyl-C₁-C₄alkyl, di(C₁-C₄alkyl)aminocarbonylC₁-C₄haloalkyl, C₁-C₄alkylsulfonyl-C₁-C₄alkyl, C₁-C₄alkoxy-C₁-C₄alkyl or C₁-C₄alkylthio-C₁-C₄alkyl;

11. A compound of formula I according to claim 1, wherein
R₁ is C₁-C₄alkyl, preferably ethyl;
R₂ is C₁-C₄haloalkyl, preferably trifluoromethyl;
X₁ is NR₃; wherein R₃ is C₁-C₄alkyl; preferably methyl;
A is CH or N;
X is S or SO₂; and
R₄ is oxetanyl which can be mono-substituted by C₁-C₄alkyl, or is C₁-C₄alkyl which can be substituted by cyano, or is C₃-C₆alkynyl, C₃-C₆cycloalkyl-C₁-C₄alkyl, di(C₁-C₄alkyl)aminocarbonylC₁-C₄haloalkyl, C₁-C₄alkylsulfonyl-C₁-C₄alkyl, C₁-C₄alkoxy-C₁-C₄alkyl or C₁-C₄alkylthio-C₁-C₄alkyl.

12. A pesticidal composition, which comprises at least one compound of formula I according to claim 1 or, where appropriate, a tautomer thereof, in each case in free form or in agrochemically utilizable salt form, as active ingredient and at least one auxiliary.

13. A method for controlling pests, which comprises applying a composition according to claim 12 to the pests or their environment with the exception of a method for treatment of the human or animal body by surgery or therapy and diagnostic methods practised on the human or animal body.

14. A method for the protection of plant propagation material from the attack by pests, which comprises treating the propagation material or the site, where the propagation material is planted, with a composition according to claim 12.
